SEQUENCE LISTING (61 sequences)

SEQ ID NO: 1 - DNA, 20 bp, Artificial Sequence
  Description of Artificial Sequence Synthetic oligonucleotide
aatgatacggcgaccaccga

SEQ ID NO: 2 - DNA, 24 bp, Artificial Sequence
  Description of Artificial Sequence Synthetic oligonucleotide
caagcagaagacggcatacgagat

SEQ ID NO: 3 - DNA, 33 bp, Artificial Sequence
  Description of Artificial Sequence Synthetic oligonucleotide
acactctttccctacacgacgctcttccgatct

SEQ ID NO: 4 - DNA, 34 bp, Artificial Sequence
  Description of Artificial Sequence Synthetic oligonucleotide
gtgactggagttcagacgtgtgctcttccgatct

SEQ ID NO: 5 - DNA, 34 bp, Artificial Sequence
  Description of Artificial Sequence Synthetic oligonucleotide
gtctcgtgggctcggagatgtgtataagagacag

SEQ ID NO: 6 - DNA, 36 bp, Artificial Sequence
  Description of Artificial Sequence Synthetic oligonucleotide
ggagttcagacgtgtgctcttccgatcttggtatag

SEQ ID NO: 7 - DNA, 38 bp, Artificial Sequence
  Description of Artificial Sequence Synthetic oligonucleotide
cgctataccaagatcggaagagcacacgtctgaactcc

SEQ ID NO: 8 - DNA, 36 bp, Artificial Sequence
  Description of Artificial Sequence Synthetic oligonucleotide
ggagttcagacgtgtgctcttccgatctggatggtt

SEQ ID NO: 9 - DNA, 38 bp, Artificial Sequence
  Description of Artificial Sequence Synthetic oligonucleotide
cgaaccatccagatcggaagagcacacgtctgaactcc

SEQ ID NO: 10 - DNA, 36 bp, Artificial Sequence
  Description of Artificial Sequence Synthetic oligonucleotide
ggagttcagacgtgtgctcttccgatctatagatta

SEQ ID NO: 11 - DNA, 38 bp, Artificial Sequence
  Description of Artificial Sequence Synthetic oligonucleotide
cgtaatctatagatcggaagagcacacgtctgaactcc

SEQ ID NO: 12 - DNA, 36 bp, Artificial Sequence
  Description of Artificial Sequence Synthetic oligonucleotide
ggagttcagacgtgtgctcttccgatctgaagaggt

SEQ ID NO: 13 - DNA, 38 bp, Artificial Sequence
  Description of Artificial Sequence Synthetic oligonucleotide
cgacctcttcagatcggaagagcacacgtctgaactcc

SEQ ID NO: 14 - DNA, 36 bp, Artificial Sequence
  Description of Artificial Sequence Synthetic oligonucleotide
ggagttcagacgtgtgctcttccgatctgagatttg

SEQ ID NO: 15 - DNA, 38 bp, Artificial Sequence
  Description of Artificial Sequence Synthetic oligonucleotide
cgcaaatctcagatcggaagagcacacgtctgaactcc

SEQ ID NO: 16 - DNA, 36 bp, Artificial Sequence
  Description of Artificial Sequence Synthetic oligonucleotide
ggagttcagacgtgtgctcttccgatctggaggttg

SEQ ID NO: 17 - DNA, 38 bp, Artificial Sequence
  Description of Artificial Sequence Synthetic oligonucleotide
cgcaacctccagatcggaagagcacacgtctgaactcc

SEQ ID NO: 18 - DNA, 36 bp, Artificial Sequence
  Description of Artificial Sequence Synthetic oligonucleotide
ggagttcagacgtgtgctcttccgatctattaaagg

SEQ ID NO: 19 - DNA, 38 bp, Artificial Sequence
  Description of Artificial Sequence Synthetic oligonucleotide
cgcctttaatagatcggaagagcacacgtctgaactcc

SEQ ID NO: 20 - DNA, 36 bp, Artificial Sequence
  Description of Artificial Sequence Synthetic oligonucleotide
ggagttcagacgtgtgctcttccgatcttttggtat

SEQ ID NO: 21 - DNA, 38 bp, Artificial Sequence
  Description of Artificial Sequence Synthetic oligonucleotide
cgataccaaaagatcggaagagcacacgtctgaactcc

SEQ ID NO: 22 - DNA, 36 bp, Artificial Sequence
  Description of Artificial Sequence Synthetic oligonucleotide
ggagttcagacgtgtgctcttccgatctaagaggtt

SEQ ID NO: 23 - DNA, 38 bp, Artificial Sequence
  Description of Artificial Sequence Synthetic oligonucleotide
cgaacctcttagatcggaagagcacacgtctgaactcc

SEQ ID NO: 24 - DNA, 36 bp, Artificial Sequence
  Description of Artificial Sequence Synthetic oligonucleotide
ggagttcagacgtgtgctcttccgatcttataatag

SEQ ID NO: 25 - DNA, 38 bp, Artificial Sequence
  Description of Artificial Sequence Synthetic oligonucleotide
cgctattataagatcggaagagcacacgtctgaactcc

SEQ ID NO: 26 - DNA, 36 bp, Artificial Sequence
  Description of Artificial Sequence Synthetic oligonucleotide
ggagttcagacgtgtgctcttccgatctttggtatt

SEQ ID NO: 27 - DNA, 38 bp, Artificial Sequence
  Description of Artificial Sequence Synthetic oligonucleotide
cgaataccaaagatcggaagagcacacgtctgaactcc

SEQ ID NO: 28 - DNA, 36 bp, Artificial Sequence
  Description of Artificial Sequence Synthetic oligonucleotide
ggagttcagacgtgtgctcttccgatctaaagtgtt

SEQ ID NO: 29 - DNA, 38 bp, Artificial Sequence
  Description of Artificial Sequence Synthetic oligonucleotide
cgaacactttagatcggaagagcacacgtctgaactcc

SEQ ID NO: 30 - DNA, 36 bp, Artificial Sequence
  Description of Artificial Sequence Synthetic oligonucleotide
ggagttcagacgtgtgctcttccgatctgagtgtgt

SEQ ID NO: 31 - DNA, 38 bp, Artificial Sequence
  Description of Artificial Sequence Synthetic oligonucleotide
cgacacactcagatcggaagagcacacgtctgaactcc

SEQ ID NO: 32 - DNA, 36 bp, Artificial Sequence
  Description of Artificial Sequence Synthetic oligonucleotide
ggagttcagacgtgtgctcttccgatcttggttaag

SEQ ID NO: 33 - DNA, 38 bp, Artificial Sequence
  Description of Artificial Sequence Synthetic oligonucleotide
cgcttaaccaagatcggaagagcacacgtctgaactcc

SEQ ID NO: 34 - DNA, 36 bp, Artificial Sequence
  Description of Artificial Sequence Synthetic oligonucleotide
ggagttcagacgtgtgctcttccgatctaaggatta

SEQ ID NO: 35 - DNA, 38 bp, Artificial Sequence
  Description of Artificial Sequence Synthetic oligonucleotide
cgtaatccttagatcggaagagcacacgtctgaactcc

SEQ ID NO: 36 - DNA, 36 bp, Artificial Sequence
  Description of Artificial Sequence Synthetic oligonucleotide
ggagttcagacgtgtgctcttccgatctgtttagta

SEQ ID NO: 37 - DNA, 38 bp, Artificial Sequence
  Description of Artificial Sequence Synthetic oligonucleotide
cgtactaaacagatcggaagagcacacgtctgaactcc

SEQ ID NO: 38 - DNA, 36 bp, Artificial Sequence
  Description of Artificial Sequence Synthetic oligonucleotide
ggagttcagacgtgtgctcttccgatcttaaatgat

SEQ ID NO: 39 - DNA, 38 bp, Artificial Sequence
  Description of Artificial Sequence Synthetic oligonucleotide
cgatcatttaagatcggaagagcacacgtctgaactcc

SEQ ID NO: 40 - DNA, 36 bp, Artificial Sequence
  Description of Artificial Sequence Synthetic oligonucleotide
ggagttcagacgtgtgctcttccgatcttagatagg

SEQ ID NO: 41 - DNA, 38 bp, Artificial Sequence
  Description of Artificial Sequence Synthetic oligonucleotide
cgcctatctaagatcggaagagcacacgtctgaactcc

SEQ ID NO: 42 - DNA, 36 bp, Artificial Sequence
  Description of Artificial Sequence Synthetic oligonucleotide
ggagttcagacgtgtgctcttccgatctgatgggtt

SEQ ID NO: 43 - DNA, 38 bp, Artificial Sequence
  Description of Artificial Sequence Synthetic oligonucleotide
cgaacccatcagatcggaagagcacacgtctgaactcc

SEQ ID NO: 44 - DNA, 36 bp, Artificial Sequence
  Description of Artificial Sequence Synthetic oligonucleotide
ggagttcagacgtgtgctcttccgatctggtagagt

SEQ ID NO: 45 - DNA, 38 bp, Artificial Sequence
  Description of Artificial Sequence Synthetic oligonucleotide
cgactctaccagatcggaagagcacacgtctgaactcc

SEQ ID NO: 46 - DNA, 36 bp, Artificial Sequence
  Description of Artificial Sequence Synthetic oligonucleotide
ggagttcagacgtgtgctcttccgatctatgtaata

SEQ ID NO: 47 - DNA, 38 bp, Artificial Sequence
  Description of Artificial Sequence Synthetic oligonucleotide
cgtattacatagatcggaagagcacacgtctgaactcc

SEQ ID NO: 48 - DNA, 36 bp, Artificial Sequence
  Description of Artificial Sequence Synthetic oligonucleotide
ggagttcagacgtgtgctcttccgatctggtgtagt

SEQ ID NO: 49 - DNA, 38 bp, Artificial Sequence
  Description of Artificial Sequence Synthetic oligonucleotide
cgactacaccagatcggaagagcacacgtctgaactcc

SEQ ID NO: 50 - DNA, 36 bp, Artificial Sequence
  Description of Artificial Sequence Synthetic oligonucleotide
ggagttcagacgtgtgctcttccgatctataaagat

SEQ ID NO: 51 - DNA, 38 bp, Artificial Sequence
  Description of Artificial Sequence Synthetic oligonucleotide
cgatctttatagatcggaagagcacacgtctgaactcc

SEQ ID NO: 52 - DNA, 36 bp, Artificial Sequence
  Description of Artificial Sequence Synthetic oligonucleotide
ggagttcagacgtgtgctcttccgatcttgggatat

SEQ ID NO: 53 - DNA, 38 bp, Artificial Sequence
  Description of Artificial Sequence Synthetic oligonucleotide
cgatatcccaagatcggaagagcacacgtctgaactcc

SEQ ID NO: 54 - DNA, 33 bp, Artificial Sequence
  Description of Artificial Sequence Synthetic oligonucleotide
ggagttcagacgtgtgctcttccgatctddddd

SEQ ID NO: 55 - DNA, 38 bp, Artificial Sequence
  Description of Artificial Sequence Synthetic oligonucleotide
cghhhhhhhhagatcggaagagcacacgtctgaactcc

SEQ ID NO: 56 - DNA, 27 bp, Artificial Sequence
  Description of Artificial Sequence Synthetic primer
  modified_base (22)..(27): a, c, t, g, unknown or other
tacacgacgctcttccgatctnnnnnn

SEQ ID NO: 57 - DNA, 59 bp, Artificial Sequence
  Description of Artificial Sequence Synthetic primer
  modified_base (25)..(32): This region may encompass one of the following sequences “AGCATGGA” or “AGGATCTA” or “ATTCCTCT” or “CAATAGTC” or “CGCTATGT” or “GATATCCA” or “GGTCCAGA” or “TCGCTAGA” or “TCTGCAAG” or “TTCGCTGA”
caagcagaagacggcatacgagatnnnnnnnngtgactggagttcagacgtgtgctctt

SEQ ID NO: 58 - DNA, 58 bp, Artificial Sequence
  Description of Artificial Sequence Synthetic primer
aatgatacggcgaccaccgagatctacactctttccctacacgacgctcttccgatct

SEQ ID NO: 59 - DNA, 69 bp, Artificial Sequence
  Description of Artificial Sequence Synthetic primer
  modified_base (29)..(36): This region may encompass one of the following sequences “GCCAAGAA” or “GGAGAGTA” or “CAATGCTA” or “TCTGTGAA”
aatgatacggcgaccaccgagatctcacnnnnnnnnacactctttccctacacgacgctcttccgatct

SEQ ID NO: 60 - DNA, 13 bp, Artificial Sequence
  Description of Artificial Sequence Synthetic oligonucleotide
gctcttccgatct

SEQ ID NO: 61 - DNA, 42 bp, Artificial Sequence
  Description of Artificial Sequence Synthetic oligonucleotide
  modified_base (1)..(8): This region may encompass one of the following sequences “AGCATGGA” or “AGGATCTA” or “ATTCCTCT” or “CAATAGTC” or “CGCTATGT” or “GATATCCA” or “GGTCCAGA” or “TCGCTAGA” or “TCTGCAAG” or “TTCGCTGA”
nnnnnnnngtctcgtgggctcggagatgtgtataagagacag

(19) United States
(12) Patent Application Publication
(10) Pub. No.: US 2022/0282325 A1
(43) Pub. Date: Sep. 8, 2022

(54) METHYLATION ASSAYS AND USES THEREOF

(71) Applicants: The General Hospital Corporation, Boston, MA (US); President and Fellows of Harvard College, Cambridge, MA (US)

(72) Inventors: Bradley BERNSTEIN, Boston, MA (US); Sarah SHAREEF, Cambridge, MA (US); Volker HOVESTADT, Boston, MA (US)

(73) Assignees: The General Hospital Corporation, Boston, MA (US); President and Fellows of Harvard College, Cambridge, MA (US)

(21) Appl. No.: 17/743,041

(22) Filed: May 12, 2022

Related U.S. Application Data
(63) Continuation of application No. PCT/US2020/060470, filed on Nov. 13, 2020.
(60) Provisional application No. 62/934,802, filed on Nov. 13, 2019.

(51) Int. Cl. C12Q 1/6869; C12Q 1/686; C12Q 1/6876

(52) U.S. Cl. CPC C12Q 1/6869; C12Q 1/686; C12Q 1/6876; C12Q 2600/154

(57) ABSTRACT

The present invention features compositions and methods for assaying DNA methylation.

### CROSS-REFERENCE TO RELATED APPLICATION

This application is a continuation application, pursuant to 35 U.S.C. § 111(a) of PCT International Application No. PCT/US2020/060470, filed Nov. 13, 2020 designating the United States and published in English, which claims the benefit of and priority to U.S. Provisional Application No.: 62/934,802, filed Nov. 13, 2019, the entire contents of each of which are incorporated herein by reference in their entirety.

### STATEMENT OF RIGHTS TO INVENTIONS MADE UNDER FEDERALLY SPONSORED RESEARCH

This invention was made with government support under Grant Nos. CA216873 and GM007753 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Dec. 22, 2020, is named 167741_022201_PCT_SL.txt and is 15,471 bytes in size.

### BACKGROUND OF THE INVENTION

DNA methylation is an important epigenetic mechanism. It is involved in many biological processes, such as development, transcriptional regulation, genome stability, aging, and disease (e.g., cancer biology), among others. CpG density is bimodal, depleted from most of the genome, with the exception of high density CpG islands that are frequently promoter-associated. DNA methylation at promoter-associated CpG islands is associated with gene silencing and intricately regulated to guide complex biological processes such as embryogenesis, aging, and tumorigenesis. Beyond promoters, DNA methylation patterns at genomic regulatory elements have been implicated in determining cell identity and chromatin structure, especially at enhancers and CCCTC-Binding factor (CTCF) regions.

Whole genome and reduced-representation methods have clarified biological roles of DNA methylation, but do not efficiently survey the vast numbers of noncoding regulatory elements in mammalian genomes. The study of dynamic DNA methylation remains difficult for a number of reasons. For example, multiple factors regulate DNA methylation, such as active enzymatic regulation and passive loss through iterative cell division. Additionally, heterogeneity within the population and low resolution attained in bulk data are obstacles that must be overcome or avoided to obtain a better understanding of this epigenetic mechanism. Current technologies use bisulfite conversion to change unmethylated cytosines to uracils while leaving methylated cytosines undisturbed for further analysis. However, bisulfite conversion is a harsh chemical reaction that can result in sample degradation, and methylation analysis remains expensive and labor intensive.

The gold standard method for reading CpG methylation is to convert unmethylated cytosines to uracil with bisulfite treatment prior to sequencing. Whole genome bisulfite sequencing (whole genome bisulfite sequencing (WGBS)) provides coverage of the entire genome, but is inefficient because vast CpG-depleted regions consume sequencing capacity. In contrast, reduced representation bisulfite sequencing (reduced representation bisulfite sequencing (RRBS)) uses restriction digest to enrich for CpG dense regions and thus provides high coverage of a fraction of the genome at reduced sequencing cost. However, reduced representation bisulfite sequencing (RRBS) lacks coverage of enhancer regions and CTCF binding sites that are outside of CpG islands. Methylation arrays that capture established regions of interest, typically promoters and CpG islands, are not ideal for profiling regulatory elements, which are of high interest.

Thus, improved methods are needed for comprehensive profiling of DNA methylation to better understand cell state dynamics. Moreover, there is a need for a strategy for profiling DNA methylation across promoters, enhancers, and CTCF sites that is efficient and compatible with low input samples and single cells.

### SUMMARY OF THE INVENTION

As described below, the present invention features compositions and methods for assaying DNA methylation in a single cell.

In one aspect of the present invention, a method is provided for polynucleotide methylation profiling, the method involving (a) contacting a double stranded polynucleotide with an endonuclease and a ligase in the presence of a double stranded adapter, where the adapter top strand contains a barcode, at least a partial sequencing primer binding site, and one or more methylated cytosines, and the bottom strand of the adapter is at least partially complementary to the top strand, and further contains a 5′ overhang, and a first member of a binding pair, where the contacting generates an adapter ligation product; (b) converting cytosines present in the adapter ligation product to uracils and isolating the adapter ligation product by contacting the first member of a binding pair with a second member of a binding pair; (c) contacting the adapter ligation product of (b) with a polymerase and one or more primers containing a random sequence and at least a partial sequencing primer binding site, thereby generating linear amplicons; (d) contacting the linear amplicons of (c) with a polymerase and forward and reverse amplification primers, thereby generating amplicons; and (e) characterizing the amplicons of (d), thereby generating a polynucleotide methylation profile.

In some embodiments, the step involving contacting the double stranded polynucleotide with an endonuclease and a ligase in the presence of a double stranded adapter is used to prepare adapter ligation products from two or more distinct double stranded polynucleotides each being from a distinct biological sample. In some embodiments, the adapter ligation products are pooled prior to converting cytosines present in the adapter ligation product to uracils. In various embodiments, characterizing the amplicons involves using the barcode to identify the biological sample corresponding to each amplicon. In various embodiments, the bottom strand of the adapter is unphosphorylated.

Another aspect of the present invention provides a method for characterizing functionally-relevant genomic regions using polynucleotide methylation profiling. The method involves (a) generating a polynucleotide methylation profile according to the above aspect, where the double stranded polynucleotide contains genomic DNA from a cell. The method further involves (b) determining a methylation state of a functionally-relevant genomic region of the genomic DNA, where the functionally-relevant genomic region is selected from any one or more of promoters, enhancers, CTCF binding sites, and CpG islands. The method also involves (c) characterizing the functionally-relevant genomic regions based upon the methylation state thereof.

In some embodiments, step (c) involves predicting chromatin structure of a functionally-relevant genomic region based upon the methylation state thereof. In some embodiments, predicting the chromatin structure comprises predicting whether the functionally-relevant genomic region comprises an active transcript, facultative heterochromatin, or constitutive heterochromatin. In some embodiments, predicting the chromatin structure comprises predicting whether the functionally-relevant genomic region comprises H3K36me3, H3K27me3, or H3K9me3. In some embodiments, characterizing the functionally-relevant genomic region comprises predicting CTCF binding. In some embodiments, characterizing the functionally-relevant genomic region comprises predicting enhancer activity. Another aspect of the present invention features a method for analyzing genetic variability within a cell population. The method involves (a) preparing DNA methylation profiles for single cells according to the method of any one of the above aspects, where each of the single cells is from the same cell line. The method further involves (b) comparing the DNA methylation profiles to determine: (i) genetic copy-number variations among the cells, and/or
(ii) variability in DNA methylation among the cells.

Another aspect of the present invention provides a method for single cell genomic DNA methylation profiling, the method involving (a) contacting DNA isolated from a single cell with a restriction enzyme and a ligase in the presence of a double stranded adapter under conditions suitable for cleavage of the double stranded polynucleotide with the restriction enzyme and ligation of the cleaved ends of the double stranded polynucleotide to the adapter, where the adapter top strand contains a cell specific barcode, at least a partial sequencing primer binding site, and one or more methylated cytosines, and the bottom strand of the adapter is at least partially complementary to the top strand, and contains a 5′ overhang, and a first member of a binding pair, thereby forming an adapter ligation product; (b) converting cytosines present in the adapter ligation product to uracils and isolating the adapter ligation product by contacting the first member of a binding pair with a second member of a binding pair; (c) contacting the adapter ligation product with a polymerase, a forward amplification primer containing a random hexamer fused to at least a partial sequencing primer binding site thereby generating linear amplicons; (d) contacting the linear amplicons of (c) with a polymerase and forward and reverse amplification primers, thereby generating amplicons; and (e) sequencing the amplicons of (d) to detect converted bases, where the first sequencing read starts at the random hexamer and the second read starts at the cell specific barcode, and the first C at the 5′ end is informative, thereby generating a single cell genomic DNA methylation profile.

A method is provided in another aspect of the present invention for single cell genomic DNA methylation profiling, the method involving (a) characterizing a population of cells using flow cytometry; (b) sorting the cells into individual reaction vessels; (c) contacting DNA isolated from a single cell with a restriction enzyme and a ligase in the presence of a double stranded adapter under conditions suitable for cleavage of the double stranded polynucleotide with the restriction enzyme and ligation of the cleaved ends of the double stranded polynucleotide to the adapter, where the adapter top strand containing a cell specific barcode, at least a partial sequencing primer binding site, and one or more methylated cytosines, and the bottom strand of the adapter is at least partially complementary to the top strand, and containing a 5′ overhang, and a first member of a binding pair, thereby forming an adapter ligation product; (d) converting cytosines present in the adapter ligation product to uracils and isolating the adapter ligation product by contacting the first member of a binding pair with a second member of a binding pair; (e) contacting the adapter ligation product with a polymerase, a forward amplification primer containing a random hexamer fused to at least a partial sequencing primer binding site thereby generating linear amplicons; (f) contacting the linear amplicons of (e) with a polymerase and forward and reverse amplification primers, thereby generating amplicons; and (g) sequencing the amplicons of (f) to detect converted bases, where the first sequencing read starts at the random hexamer and the second read starts at the cell specific barcode, and the first C at the 5′ end is informative, thereby generating a single cell genomic DNA methylation profile. In some embodiments, the cells are characterized using antibodies. In some embodiments, the individual reaction vessel is a well, droplet, tube, or microfluidic chamber. In some embodiments, the population of cells is derived from a subject. In some embodiments, the population of cells is derived from a biological sample of a subject.

Another aspect provides a method for characterizing DNA methylation of a subject having or suspected of having a disease, the method involving (a) characterizing a population of cells using flow cytometry; (b) sorting the cells into individual reaction vessels; (c) contacting DNA isolated from a single cell with restriction enzyme and a ligase in the presence of a double stranded adapter under conditions suitable for cleavage of the double stranded polynucleotide with the restriction enzyme and ligation of the cleaved ends of the double stranded polynucleotide to the adapter, where the adapter top strand containing a cell specific barcode, at least a partial sequencing primer binding site, and one or more methylated cytosines, and the bottom strand of the adapter is at least partially complementary to the top strand, and contains a 5′ overhang, and a first member of a binding pair, thereby forming an adapter ligation product; (d) converting cytosines present in the adapter ligation product to uracils and isolating the adapter ligation product by contacting the first member of a binding pair with a second member of a binding pair; (e) contacting the adapter ligation product with a polymerase, a forward amplification primer containing a random hexamer fused to at least a partial sequencing primer binding site thereby generating linear amplicons; (f) contacting the linear amplicons of (e) with a polymerase and forward and reverse amplification primers, thereby generating amplicons; and (g) sequencing the amplicons of (f) to detect converted bases, where the first sequencing read starts at the random hexamer and the second read starts at the cell specific barcode, and the first C at the 5′ end is informative, thereby characterizing DNA methylation of the subject. In some embodiments, the subject has or is suspected of having a disease associated with an alteration in DNA methylation. In some embodiments, the disease is cancer or an imprinting disorder. In some embodiments, the cancer is acute myeloid leukemia (AML), chronic lymphocytic leukemia (CLL), glioma, or chondrosarcoma. In some embodiments, the imprinting disorder is Angelman syndrome (AS) or Prader-Willi syndrome (PWS). In some embodiments, the disease is Lynch Syndrome or age related clonal hematopoiesis (ARCH). In some embodiments, the amplicons are sequenced using the Illumina NextSeq 500 platform.

Another aspect provides a method for polynucleotide methylation profiling, the method involving (a) contacting a double stranded polynucleotide with a transposase in the presence of a double stranded adapter, where the adapter top strand contains a barcode, at least a partial transposase binding site, and one or more methylated cytosines, and the bottom strand of the adapter is at least partially complementary to the top strand, where the contacting generates an adapter transposition product; (b) converting cytosines present in the adapter transposition product to uracils; (c) contacting the adapter transposition product of (b) with a polymerase and a primer containing a random sequence, a barcode, and at least a partial sequencing primer binding site, thereby generating linear amplicons; (d) contacting the linear amplicons of (c) with a polymerase and forward and reverse amplification primers, thereby generating amplicons; and (e) characterizing the amplicons of (d), thereby generating a polynucleotide methylation profile.

In some embodiments of the foregoing aspects, the double stranded polynucleotide is DNA. In some embodiments, the forward amplification primer contains a random hexamer fused to at least a partial sequencing primer binding site. In some embodiments, the reverse primer contains a barcode and at least a partial sequencing primer binding site. In some embodiments, a first sequencing read starts at the random hexamer and the second read starts at the cell specific barcode, and the first C at the 5′ end is informative. In some embodiments, the endonuclease activity is dependent on the methylation state of a recognition sequence. In some embodiments, the endonuclease is MspI, ScaI, BamHI, HindIII, NotI, and SpeI. In some embodiments, the bottom strand of the adapter does not contain a methylated cytosine. In some embodiments, the polynucleotide is isolated from a cell in vivo or in vitro. In some embodiments, the first member of the binding pair is biotin and the second member of the binding pair is streptavidin. In some embodiments, the first or second member of the binding pair is fixed to a substrate. In some embodiments, the substrate is selected from any one or more of a bead, a membrane, a chip, and a slide. In some embodiments, the polymerase is a strand-displacing polymerase. In some embodiments, the strand displacing polymerase is Klenow exo− or Bst DNA polymerase. In some embodiments, the partial sequencing primer binding site is SBS3, SBS12, P5, or P7. In some embodiments, the ligase is T4 ligase.

In any of the above aspects, the hexamer extensions are used to characterize an amplicon as a PCR duplicate.

A method is also provided for bisulfite conversion of a nucleic acid molecule, the method involving contacting a polynucleotide with a double-stranded adapter containing a top strand containing one or more methylated cytosines and a bottom strand that lacks methylated cytosines, an MspI enzyme, and a ligase to form a nucleic acid fragment having adapters at its termini; and contacting the nucleic acid fragment with bisulfite to form a converted nucleic acid fragment.

In any of the above aspects, the step involving contacting DNA isolated from a single cell with restriction enzyme and a ligase in the presence of a double stranded adapter is used to prepare adapter ligation products from DNA from two or more cells and the adapter ligation products are pooled prior to the step involving converting cytosines present in the adapter ligation product to uracils. In any of the above aspects, the method involves an analysis, which involves using the barcode to identify the cell corresponding to each amplicon. In any of the above aspects, the method involves an analysis, and the analysis involves characterizing an amplicon as corresponding to a cell based upon a single nucleotide polymorphism unique to the cell. In any of the above aspects, the bottom strand of the adapter is unphosphorylated. In any of the above aspects, the cell was exposed to a stimulus prior to preparing the polynucleotide methylation profile. In some embodiments, the stimulus comprises contacting the cell with an agent.

In any of the above aspects, the cell is associated with a disease. In any of the above aspects, the disease is cancer, autoimmune disease, Angelman syndrome (AS), Prader-Willi syndrome (PWS), Lynch syndrome, or age related clonal hematopoiesis (ARCH). In some embodiments, the cancer is bladder, bone, breast, colon, esophageal, glioblastoma, leukemia, liver, lung, ovarian, prostate, or thyroid cancer.

Compositions and articles defined by the invention were isolated or otherwise manufactured in connection with the examples provided below. Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, “adapter” refers to a nucleic acid molecule that can be ligated to the end of a DNA or RNA molecule. In one embodiment, an adapter is recognized by a primer used in a sequencing reaction or sequencing platform. In some embodiments, the sequencing platform is an Illumina sequencing platform. In some embodiments, the adapter comprises a barcode or other identifying nucleic acid sequence.

By “agent” is meant any small molecule chemical compound, antibody, nucleic acid molecule, or polypeptide, or fragments thereof.

By “alteration” is meant a change (increase or decrease) in the sequence, methylation state, expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “amplicon”, is meant a polynucleotide sequence that is the source and/or product of the amplification of another polynucleotide sequence. In some embodiments, the amplicon is a product of a polymerase chain (PCR) reaction used to amplify a polynucleotide sequence.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

“Biological sample” as used herein refers to a cell or to a sample obtained from a biological subject, including a sample of a biological tissue or fluid origin, obtained or collected in vivo or in situ, that contains genomic DNA. A biological sample also includes samples from a region of a biological subject containing precancerous or cancer cells or tissues. Such samples can be, but are not limited to, organs, tissues, fractions and cells isolated from mammals including, humans such as a patient, mice, and rats. Biological samples also may include sections of the biological sample including tissues, for example, frozen sections taken for histologic purposes. A biological sample in various embodiments is of an eukaryotic origin, for example, insects, protozoa, birds, fish, reptiles, and preferably a mammal, for example, rat, mouse, cow, dog, guinea pig, or rabbit, and more preferably a primate, for example, chimpanzees or humans. Non-limiting examples of cell lines to which the cell may belong include a K562 cell, a Kasumi-1 cell, an OCI-AML3 cell, an HL-60 cell, a primary T-cell, an H1 embryonic stem cell, a primary mammary epithelial cell, an IMR90 fibroblast cell, or a GM12878 cell. By “cell line” is meant cells with a substantially uniform genetic makeup and descended from a single cell.

By “barcode” is meant a portion of a nucleotide sequence that provides for molecular identification of the sequence. For example, a barcode sequence enables the segregation of sequence reads thereby allowing identification of the source of the sequence (e.g., a particular cell).

By “binding pair” is meant two molecules that specifically bind to each other, and that do not specifically bind to other molecules. For example, biotin and streptavidin, an antibody and a molecule having an epitope that specifically binds the antibody, and the like are binding pairs.

By “chromatin” is meant a complex of DNA and protein found in cells. In various embodiments, chromatin comprises histones.

By “chromatin structure” is meant the state of folding of chromatin (e.g., heterochromatin or euchromatin) and/or the proteins and nucleotides comprising the chromatin and states of modification of the proteins and nucleotides.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “CpG island” is meant a region of a genome where a cytosine nucleotide is followed by a guanine nucleotide, or vice versa, in the linear sequence of bases at a high frequency. In various embodiments, a CpG island is a region of a genome comprising a least about or about 50 bp, 100 bp, 150 bp, 200 bp, or 250 bp, and with a GC percentage of greater than about 25%, 50%, or 75%.

By “CTCF binding site” is meant a DNA sequence recognized by transcriptional repressor CTCF. In various embodiments, CTCF is alternatively referred to as CCCTC-binding factor. In some embodiments, a CTCF binding site comprises three regularly spaced repeats each comprising the sequence CCCTC.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include cancers (e.g., acute myeloid leukemia (AML), chronic lymphocytic leukemia (CLL), glioma, and chondrosarcoma. In some embodiments, the disease is Lynch Syndrome, an inherited disease resulting primarily in colorectal cancer. In some embodiments, the disease is age related clonal hematopoiesis (ARCH; also called clonal hematopoiesis of indeterminate potential (CHIP)). In some embodiments, the disease is an imprinting disease such as Angelman syndrome (AS) or Prader-Willi syndrome (PWS).

By “enhancer” is meant a region of DNA sufficient to increase the likelihood that transcription of a particular gene will occur. In various embodiments, an enhancer is a region of DNA that can be bound by proteins that, when bound to the enhancer, increase transcription rates of a gene associated with the enhancer.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

The term “Next Generation Sequencing (NGS)” refers to massive parallel sequencing of clonally amplified molecules or single nucleic acid molecules. “Massive parallel sequencing” refers to simultaneously performing more than 1000 separate sequencing reactions. Non-limiting examples of NGS include sequencing-by-synthesis using reversible dye terminators, sequencing-by-ligation, and electronic detection sequencing methods. In some embodiments, NGS is carried out using the Illumina NextSeq 500 platform. Electronic detection sequencing methods include those used in the Ion Torrent sequencing strategy (ThermoFisher Scientific) or MiSeq platform (Illumina), wherein changes in pH are detected when a nucleotide is incorporated into a nucleic acid strand resulting in release of a hydrogen ion.

As used herein, “obtaining” as in “obtaining an agent” includes synthesizing, purchasing, or otherwise acquiring the agent.

By “PCR duplicate” is meant a sequence read that results from sequencing two or more copies of the exact same adapter ligation product.

By “promoter” is meant a polynucleotide sufficient to direct transcription.

By “reduces” is meant a negative alteration of at least 10%, 25%, 50%, 75%, or 100%.

By “reference” is meant a standard or control condition.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “single nucleotide polymorphism (SNP)” is meant a variation at a single position in a DNA sequence among a population of cells sharing a common genetic makeup. In some embodiments, a single nucleotide polymorphism can be used to identify a cell.

By “subject” is meant a human or non-human mammal, such as a bovine, equine, canine, ovine, or feline. In some embodiments, “subject” refers to any domesticated animal.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a diagram showing hypermethylation and CTCF disruption due to a mutated IDH protein.

FIGS. 2A-2H show that the methylation assay described herein is an enhancer enriching methylation assay. FIG. 2A is a diagram depicting sorting individual cells into individual wells of a 96-well plate containing lysis reagents. FIG. 2B provides an overview of the method. FIG. 2C is a diagram depicting the simultaneous digestion of genomic DNA and ligation of adapters to the termini of the digested DNA. The biotin labels of the adapters bind to streptavidin beads. “P” denotes phosphate, and “B” denotes biotin. FIG. 2D is a diagram showing the Klenow exo− mediated linear amplification of the adapter-bound digested DNA after bisulfite conversion. For FIGS. 2C and 2D the dots in the adapter represent methylated cytosines. FIG. 2E is a diagram depicting the Library PCR and Final Library PCR amplifications used to generate a library of fragmented DNA. The library represents the bisulfite converted regions of genomic DNA in the cell. FIG. 2F is a diagram of method of generating a library of fragmented DNA. This method uses Tn5-mediated transposition to attach adapters to genomic DNA rather than simultaneous digestion and ligation before bisulfite conversion. The “Red Image Green Image” illustrates accelerated detection of all four DNA bases on the NextSeq 500 System using only two images to capture red and green filter wavelength bands. A bases will be present in both images (yellow cluster), C bases in red only, T bases in green only, and G bases in neither. FIGS. 2G and 2H depict dual indexed sequencing work flows.

FIG. 3 is a graph showing aberrant DNA methylation in different regulatory regions of genes, including enhancers.

FIG. 4 is a diagram showing that enhancer hypomethylation is associated with target gene activation. “TF” denotes transcription factor; “TET” denotes ten eleven translocation, an enzyme that demethylates DNA.

FIGS. 5A-5C characterize the ability of the methods of the present invention to identify enhancers. FIG. 5A is a graph comparing the number of enhancers detected using the methods of the present invention to the number of enhancers detected using a reduced representation bisulfite sequencing approach (RRBS) with downsampling. FIG. 5B is a graph comparing the number of enhancers detected using the methods of the present invention to the number of enhancers detected using a RRBS approach. FIG. 5C comprises pie charts showing base pairs sequenced and enhancers covered. The term CCRE refers to candidate cis-regulatory elements.

FIGS. 6A-6D illustrate the robustness of the methods of the present invention when using low inputs (i.e., 1000 cells, 10 ng, or 100 cells). FIG. 6A is a graph showing a principal component analysis of methylation profiles of AML cell lines. Three acute myeloid leukemia cell lines (HL60, Kasumi, and OCI-AML3) were assayed. Data for each input level (i.e., 1000 cells, 10 ng, or 100 cells) clusters with the data for the other input levels for each cell line. FIG. 6B is a heatmap of low input methylation profiles. Individual CpG Beta values (Spearman's correlation) are shown. The Beta value is the ratio of the methylated probe intensity and the overall intensity (sum of methylated and unmethylated probe intensities) and can be used with sequencing data generated on the Illumina platform. FIG. 6C is a graph analyzing the unique CpGs detected relative to the number of sequencing reads per single cell in single cell RRBS versus single cell methylation profiles obtained using the methods disclosed herein. FIG. 6D presents methylation profiles.

FIG. 7 is a graph showing the low cross-talk of barcodes.

FIGS. 8A-8B illustrate distinct methylation profiles of nonoverlapping CpG windows for two cell lines. FIG. 8A is a plot showing that single cell methylation profiles cluster by cell identity. PC2 is plotted versus PC1 using 100 CpG nonoverlapping windows. FIG. 8B is a plot showing the results of a two-dimensional principal component analysis illustrating clustering of the K562 and GM12878 cell lines.

FIG. 9 is a graph of a t-Distributed Stochastic Neighbor Embedding (t-SNE) analysis that shows clustering of cells carrying different mutations.

FIG. 10 provides heat maps demonstrating that methylation profiles assayed using methods described herein provide for the identification of differential promoter methylation.

FIG. 11 is a collection of heatmaps and a plot. The top panel of FIG. 11 is a collection of heatmaps demonstrating that the methylation assay described herein has higher coverage over accessible regions relative to other methods for assaying methylation. The bottom panel of FIG. 11 is a plot demonstrating that the methylation assay described herein can detect hypomethylation over CTCF motif where CTCF is bound and nucleosome periodicity.

FIG. 12 is a scatter plot of a K-nearest neighbor visualization characterizing transcription by single cell RNA sequencing of sorted cell populations in bone marrow (BM).

FIGS. 13A-13E illustrate human bone marrow methylation detection. FIG. 13A provides scatter plots demonstrating the flow sorting of T cells, progenitors, and monocytes using CD34, CD3, and CD14. FIG. 13B is a graph depicting the principal component analysis of duplicate cell methylation profiles for 100 cells indicating methyl profiles cluster by cell identity. FIG. 13C is a heatmap of the duplicate 100 cell methylation profiles depicting pairwise spearman correlation. FIG. 13D is a heatmap depicting enriched transcription factors characterized by the Kruskal-Wallis Rank Sum Test. FIG. 13E is a volcano plot of differentially methylated regions in T cells and progenitors. T cells are hypermethylated.

FIGS. 14A to 14D are a schematic, a genome plot, and heatmaps corresponding to an expanded representation DNA methylation profiling method compatible with low input samples. FIG. 14A is a schematic of expanded representation bisulfate sequencing (XRBS). Barcoding samples in a single well through sequential lysis, digestion, and ligation minimizes DNA loss. Binding biotinylated adapters to streptavidin beads enables multiple samples to be combined into a single bisulfite conversion reaction, minimizing batch effects introduced during conversion. Random hexamer-primed second strand synthesis recovers fragmented DNA. PCR amplification yields sequencing libraries. P5 is a nucleic acid molecule with the sequence AATGATACGGCGACCACCGA; P7 is a nucleic acid molecule with the sequence CAAGCAGAAGACGGCATACGAGAT; SBS3 is a nucleic acid molecule with the sequence ACACTCTTTCCCTACACGACGCTCTTCCGATCT; SBS12 is a nucleic acid molecule with the sequence GTGACTGGAGTTCAGACGTGTGCTCTTCCGATCT; i7 is an index; hex. is short for “hexamer”; R1 is a forward primer; R2 is a reverse primer; B is biotin. FIG. 14B provides heatmaps comparing individual CpG methylation values acquired by expanded representation bisulfite sequencing (XRBS) with or without biotinylated adapters (left, r=0.96), and between technical replicates (right, r=0.97) for K562 cells. Percentages indicate the fraction of CpGs that differed between conditions (difference in beta-values >0.5). Analysis limited to CpGs with at least 15-fold coverage (n=313,330 and 721,760). FIG. 14C provides a genome plot for the GFI1B gene locus compares read coverage between expanded representation bisulfite sequencing (XRBS) and public whole genome bisulfite sequencing (WGBS) and reduced representation bisulfite sequencing (RRBS) datasets for K562 cells. Boxes represent reads, and unmethylated (darker grey) and methylated (lighter grey) CpGs are indicated. CTCF and H3K27ac ChIP-seq tracks reveal functional elements. Vertical grey lines mark MspI restriction sites. Insert shows expanded representation bisulfite sequencing (XRBS) reads flanking an isolated MspI site, which is not covered by reduced representation bisulfite sequencing (RRBS). FIG. 14D provides heatmaps comparing individual CpG methylation values between reduced representation bisulfite sequencing (RRBS) and public whole genome bisulfite sequencing (WGBS) (r=0.91), reduced representation bisulfite sequencing (RRBS) (r=0.90), or EPIC methylation array (r=0.90) datasets for K562 cells. Percentages indicate the fraction of CpGs that differed between conditions (difference in beta-values >0.5). In each heatmap of FIGS. 14B and 14D, the highest fractions of covered CpGs fall near the lower-left and upper-right corners of each plot and the lowest fractions of covered CpGs covered fall near to the upper-left and lower-right corners of each plot.

FIGS. 15A to 15F are plots and heatmaps demonstrating that expanded representation bisulfite sequencing (XRBS) covers CpGs in regulatory elements. FIGS. 15A and 15B are plots showing the number of CpG islands (FIG. 15A) or promoters (FIG. 15B) with at least 100-fold combined coverage as a function of sequencing depth (x-axis) for expanded representation bisulfite sequencing (XRBS), whole genome bisulfite sequencing (WGBS), and reduced representation bisulfite sequencing (RRBS). Enrichment for functional elements at a uniform sequencing depth of 10 billion base pairs is indicated. Vertical grey line indicates break in x-axis scale. FIGS. 15C and 15D are plots showing the number of enhancers (FIG. 15C) or CTCF insulator binding sites (FIG. 15D) with at least 25-fold combined coverage as a function of sequencing depth (x-axis) for expanded representation bisulfite sequencing (XRBS), whole genome bisulfite sequencing (WGBS), and reduced representation bisulfite sequencing (RRBS). FIG. 15E is a collection of heatmaps depicting 2 kb genomic regions (rows) centered at CTCF binding sites, grouped into regions that are fully hypomethylated, and that overlap (CTCF enhancers) or do not overlap (CTCF insulators) with H3K27ac peaks. Regions are divided into 100 equally sized windows. Panels from left to right show: CTCF ChIP-seq signal, H3K27ac ChIP-seq signal, and methylation calls from 450 k methylation array, EPIC methylation array, reduced representation bisulfite sequencing (RRBS), expanded representation bisulfite sequencing (XRBS), and whole genome bisulfite sequencing (WGBS). All datasets except expanded representation bisulfite sequencing (XRBS) were retrieved from the Encyclopedia of DNA Elements (ENCODE). FIG. 15F is a plot showing average DNA methylation levels across 4 kb regions centered at CTCF insulators (black) or CTCF enhancers (grey). Periodicity of DNA methylation around CTCF binding sites is consistent with positioned nucleosomes, but much more pronounced for CTCF insulators.

FIGS. 16A to 16G are a genome plots, isometric projection plots, heatmaps, and a bar graph demonstrating that expanded representation bisulfite sequencing (XRBS) reveals variable hypomethylation in cell lines and in response to a demethylating agent. FIG. 16A provides heatmaps showing genome-wide DNA methylation in 100 kb windows across four cell lines. Windows are sorted by decreasing DNA methylation for each cell line. Average methylation for each cell line is indicated below. FIG. 16B is a bar plot showing average DNA methylation levels for CpG islands and non-CpG island regions across cell lines. FIG. 16C is an isometric projection plot positioning all 100 kb genomic windows (dots) according to their normalized signal for three histone modifications in K562 cells. Windows are shaded by average DNA methylation of non-island CpGs. Windows marked by repressive marks (H3K27me3 and H3K9me3) are generally unmethylated, while only windows marked by H3K36me3 are methylated, consistent with very low global DNA methylation in K562 cells (see also FIG. 21F). FIG. 16D is an isometric projection plot as in FIG. 16C but with windows colored by the Hi-C experiment-derived first eigenvector. The H3K36me3 and H3K27me3 histone marks overlap with compartment A, while the H3K9me3 mark overlaps with compartment B in K562 cells. FIG. 16E is a genome plot showing average DNA methylation and the Hi-C-derived first eigenvector for K562 cells for a 80 Mb genomic segment of chromosome 2. Megabase-scale hypomethylated blocks primarily overlap compartment B (light grey), whereas shorter hypomethylated regions and hypermethylated regions overlap compartment A (dark grey). FIG. 16F is a heatmap showing genome-wide DNA methylation in 100 kb windows across DMSO and decitabine treated HL-60 and OCI-AML3 cells. Windows are sorted according to DNA methylation for each treatment. Average methylation for each cell line is indicated below. FIG. 16G is a genome plot for the same region as in FIG. 16E showing the Hi-C-derived first eigenvector illustrating compartments A (dark grey) and B (light grey) for HL-60 cells, and average DNA methylation for HL-60 or OCI-AML3 treated with decitabine or control. Ratio shows methylation difference between decitabine and control (dimethyl sulfoxide (DMSO)). Decitabine reduces methylation in both compartments to a similar extent. In FIGS. 16A and 16F, the lowest portion of each heatmap corresponds to the lowest level of methylation detected and the highest portion of each heatmap corresponds to the highest level of methylation detected.

FIGS. 17A to 17C are heatmaps demonstrating that expanded representation bisulfite sequencing (XRBS) predicts regulatory states of functional elements. FIG. 17A is a heatmap depicting 8 kb genomic regions (rows) centered at transcription start sites and divided into 100 equally sized bins. Panels show average methylation from 1,000-cell XRBS profiles for the indicated cell types. Promoters (rows, ≥25-fold combined coverage in every cell line) are grouped by the cell line in which they were specifically hypermethylated (top) or hypomethylated (bottom). Hypomethylated promoters specific to K562 cells are downsampled for visualization. FIG. 17B is a heatmap depicting 8 kb regions (rows) centered on enhancers identified in K562 and OCI-AML3 ChIP-seq datasets. Rows are ordered by methylation difference between both cell lines. Panels show average methylation from 1,000-cell expanded representation bisulfite sequencing (XRBS) profiles and H3K27ac signals for K562 and OCI-AML3. DNA hypomethylation predicts enhancer H3K27ac in a given cell line. Enhancers hypomethylated in both cell lines were down-sampled for visualization. FIG. 17C is a heatmap depicting 2 kb regions (rows) centered on CTCF insulators identified in HL-60 and K562 CTCF ChIP-seq datasets. Rows are ordered by the difference in DNA methylation within peaks between both cell lines. CTCF sites with cell type-specific DNA hypermethylation are depleted for CTCF binding in the respective cell line. CTCF sites hypomethylated in both cell lines were down-sampled for visualization.

FIGS. 18A to 18J are schematics, scatterplots, plots, heat maps, and a bar plot demonstrating that single cell expanded representation bisulfite sequencing (XRBS) reveals epigenetic and genetic heterogeneity. FIG. 18A is a schematic of a single cell experimental setup. Cells were sorted into separate barcoding reactions, and then pooled for subsequent bisulfite conversion, hexamer extension, and library amplification. FIG. 18B is a schematic showing a barcoding strategy for 96 single cells from three cell lines (K562, GM12878, and Yac1) through a combination of 24 single cell barcodes and four library barcodes. FIG. 18C is a scatterplot showing for each single cell expanded representation bisulfite sequencing (XRBS) profile (dots) the number of reads that aligned specifically to the mouse genome (x-axis) versus the human genome (y-axis), confirming the absence of cross contamination between human and mouse cells prepared in the same reaction pool. FIG. 18D is a scatterplot showing for each single K562 or GM12878 cell XRBS profile (dots) coverage of homozygous SNPs specific to K562 (y-axis) or GM12878 (x-axis) cells, confirming the absence of cross contamination between the two human cell lines. FIG. 18E is a plot showing copy number variations for single K562 cells inferred from expanded representation bisulfite sequencing (XRBS) profiles. Amplification of the prototypic BCR-ABL fusion was detected. FIG. 18F is a heatmap depicting copy number variations inferred for single GM12878 (n=32, top) and K562 cells (n=27, bottom). Single cells are ordered in decreasing read coverage. Single GM12878 cells were largely copy number neutral, while single K562 cells exhibited multiple chromosomal and sub-chromosomal abnormalities. FIG. 18G is a bar plot showing the average genome-wide DNA methylation for high-quality single cell K562, GM12878, and Yac1 profiles. FIG. 18H is a plot showing t-SNE analysis of pairwise distances between high-quality single cell K562 and GM12878 profiles. FIG. 18I presents scatterplots comparing single cell average DNA methylation within H3K27me3 domains (y-axis, left) and H3K9me3 domains (right) against genome-wide average DNA methylation levels (x-axis). Diagonal lines indicate results from linear regression analysis and residuals for each single cell. FIG. 18J presents scatterplots comparing single cell average DNA methylation within various early and late replicating regions (y-axis) against genome-wide average DNA methylation levels (x-axis). The horizontal bar above each plot shows the fraction of regions associated with active and repressive histone marks (H3K36me3, H3K27me3, and H3K9me3). Diagonal lines indicate results from linear regression analysis and residuals for each single cell.

FIGS. 19A to 19D are plots and histograms presenting an evaluation of single MspI anchor design for methyl-CpG profiling. FIG. 19A presents plots showing results from an in silico MspI restriction digest analysis of the human genome. The cumulative number of MspI fragments (total of 2.3 million, left), of basepairs (total of 3.1 billion, middle), and of CpGs (total of 29.4 million, right) is shown relative to increasing MspI fragment length. Vertical dotted lines show the size range of fragments captured in a typical expanded representation bisulfite sequencing (XRBS) experiment. This analysis shows that reduced representation bisulfite sequencing (RRBS) only covers 2.4% of the genome, but enriches for 11.8% of genomic CpGs. An additional 38.7% of CpGs are located within 300 bp of a single MspI site that are not captured by reduced representation bisulfite sequencing (RRBS). FIG. 19B presents histograms showing coverage depth of MspI restriction sites for each replicate of a 10 ng expanded representation bisulfite sequencing (XRBS) library (left, middle), and both replicates combined (right). FIG. 19C is a histogram showing coverage depth of CpGs in the combined dataset of both replicates. FIG. 19D is a plot showing unique reads as a function of aligned reads in millions. With greater sequencing depth the fraction of unique reads decreases, as the chance of sampling a non-unique read (i.e. polymerase chain reaction (PCR) duplicate) increases.

FIGS. 20A to 20D are plots relating to downsampling of expanded representation bisulfite sequencing (XRBS) libraries over regulatory elements. FIG. 20A is a plot showing number of detected fragments plotted as a function of MspI fragment length from expanded representation bisulfite sequencing (XRBS) 10 ng library replicates and from public Encyclopedia of DNA Elements (ENCODE) reduced representation bisulfite sequencing (RRBS) data. FIG. 20B is a plot comparing CpG coverage as a function of sequencing depth (x-axis) for expanded representation bisulfite sequencing (XRBS) (the darker labeled line in FIGS. 20B to 20D), whole genome bisulfite sequencing (WGBS) (unlabeled line in FIGS. 20B to 20D), and reduced representation bisulfite sequencing (RRBS) (the lighter labeled line in FIGS. 20B to 20D). FIG. 20C presents a downsampling analysis plot as in FIG. 20B but restricted to CpGs within CpG islands (top panel) and gene promoters (bottom panel). FIG. 20D presents a downsampling analysis plot as in FIG. 20B but restricted to CpGs within H3K27ac enhancers (top panel) and CTCF binding sites (bottom panel).

FIGS. 21A to 21G are plots and heatmaps demonstrating a correlation of DNA methylation with histone marks and compartment calls. FIG. 21A is a plot showing unique reads as a function of aligned reads in low-coverage expanded representation bisulfite sequencing (XRBS) libraries from K562, HL-60, OCI-AML3, and Kasumi-1 cells. FIG. 21B is a plot showing unique reads as a function of aligned reads in low-coverage libraries from K562, Kasumi-1, HL-60, OCI-AML3 cells. Libraries were generated from 1,000 (dark grey) and 100 (light grey) cells sorted directly into lysis buffer. Libraries generated from 1,000 cells were comparable to libraries generated from 10 ng of purified DNA (FIG. 21A), whereas 100 cell libraries show reduced complexity. FIG. 21C presents heatmaps showing Pearson correlation of expanded representation bisulfite sequencing (XRBS) methylation profiles of 100 kb windows generated from 10 ng gDNA, 1,000 or 100 sorted cells across four cell lines. Dendrogram derived from unsupervised clustering is indicated to the left. Sample grouping by DNA methylation is consistent with cell identity, indicating low technical variability between input material. FIG. 21D presents heatmaps showing correlation between average DNA methylation values and signal for H3K9me3 (left), H3K27me3 (center), and H3K36me3 (right) in 100 kb-windows for K562 cells. FIG. 21E is a heatmap showing correlation between DNA methylation and the Hi-C-derived first eigenvector indicating compartment A (positive values) and compartment B (negative values) in 100 kb-windows for K562 cells. FIG. 21F is a collection of heatmaps showing correlation between average DNA methylation values and ChIP seq signal for H3K9me3 (top), H3K27me3 (middle), and H3K38me3 (bottom) in 100 kb-windows for human H1 embryonic stem cells, primary T cells and mammary epithelial cells, and cultured IMR90, GM12878 and K562 cells. FIG. 21G is a collection of heatmaps as in FIG. 21F, but shows correlation between average DNA methylation values and the Hi-C-derived first eigenvector (x-axis). Positive values correspond to compartment A and negative values correspond to compartment B. While hypomethylation of compartment B is most pronounced in K562 cells, a similar trend is also observed in other cultured cell lines and in primary mammary epithelial cells.

FIGS. 22A to 22F are plots, an image, a bar plot, and heatmaps presenting a characterization of decitabine treatment of cancer cell lines. FIG. 22A is a plot presenting a dose response curve for decitabine treatment of three cell lines Kasumi, HL-60, and OCI-AML3. Viability was measured using CellTiter-Glo® and is reported as luminescence relative to control dimethyl sulfoxide (DMSO) treated cells. FIG. 22B presents images showing HL60 and OCI-AML3 cells treated with 300 nM decitabine and a dimethyl sulfoxide (DMSO) vehicle control. Morphology of decitabine treated cells were similar to control. FIG. 22C is a plot showing unique reads as a function of aligned reads in expanded representation bisulfite sequencing (XRBS) libraries from dimethyl sulfoxide- (DMSO) and decitabine-treated HL-60 and OCI-AML3 cells. FIG. 22D is a bar plot showing average DNA methylation values across island (dark grey) and non-island (light grey) CpGs in dimethyl sulfoxide- (DMSO) and decitabine-treated HL-60 and OCI-AML3 cells. For example, average methylation of non-island CpGs in HL-60 cells is reduced from 68.1% to 54.3% by decitabine treatment (20.2% reduction). FIG. 22E is a heatmap showing correlation between Hi-C-derived first eigenvectors from K562 and HL-60 cell lines in 100 kb-windows, indicating high agreement in compartment structure between both cell lines. FIG. 22F is a collection of heatmaps demonstrating correlation between average DNA methylation values and Hi-C-derived eigenvector in 100 kb-windows for dimethyl sulfoxide- (DMSO) (left) and decitabine-treated HL-60 cells (center). The heatmap on the right shows relative DNA methylation values of decitabine- and dimethyl sulfoxide- (DMSO) treated cells. Despite compartment B showing lower methylation compared to compartment A at baseline, induced DNA hypomethylation with decitabine treatment affects compartment A and B equally.

FIGS. 23A to 23H are plots, scatterplots, and heatmaps showing differential DNA methylation of gene promoters, enhancers, and CTCF binding sites. FIG. 23A is a plot showing unique reads as a function of aligned reads in 1,000 cell high-coverage libraries of four cell lines. FIG. 23B is a collection of heatmaps depicting 8 kb regions (rows) centered at transcription start sites that show cell line-specific hyper- or hypomethylation (as in FIG. 17A) and divided into 100 equally sized windows. Panels from left to right show methylation calls from 450 k methylation array, reduced representation bisulfite sequencing (RRBS), expanded representation bisulfite sequencing (XRBS), and whole genome bisulfite sequencing (WGBS). All datasets except expanded representation bisulfite sequencing (XRBS) were retrieved from the Encyclopedia of DNA Elements (ENCODE). FIG. 23C is a plot showing expression levels for genes that were associated with cell line-specific promoter hyper- and hypo-methylation. Genes with an expression level larger than 0.5 were considered as expressed. Average gene expression levels are indicated by horizontal lines. P-values were generated using the Mann-Whitney U test. In K562, the majority (74.0%) of hypomethylated promoters are associated with non-expressed genes, which is unique to this cell line, consistent with global hypo-methylation in K562. FIG. 23D is a scatterplot comparing gene expression level and H3K4me3 ChIP-seq signal for gene promoters that were identified as differentially methylated between all four cell lines. Individual promoters (dots) are colored (i.e., not the lightest shade of grey) if specifically hypermethylated (medium shade of grey) and hypermethylated (darkest shade of grey) in K562 cells. This analysis showed that promoters which were not expressed and specifically hypomethylated in K562 (n=1,624) were generally negative for the H3K4me3 histone mark (98.7%), whereas promoters that were hypomethylated and expressed (n=570) were more frequently positive for H3K4me3 (45.0%). FIG. 23E is a collection of heatmaps depicting 8 kb regions (rows) centered at enhancers that are hypomethylated in K562, OCI-AML3, or both cell lines (as in FIG. 17B). Panels from left to right show methylation calls from 450 k methylation array, reduced representation bisulfite sequencing (RRBS), expanded representation bisulfite sequencing (XRBS), and whole genome bisulfite sequencing (WGBS). All datasets except expanded representation bisulfite sequencing (XRBS) were retrieved from the Encyclopedia of DNA Elements (ENCODE). FIG. 23F is a scatterplot showing merged enhancer regions from OCI-AML3 and K562 H3K27ac ChIP-seq datasets. Individual enhancers (dots) are colored (i.e., darker two shades of grey) if specifically hypermethylated (medium shade of grey) or hypomethylated (darkest shade of grey) in K562 cells. FIG. 23G is a collection of heatmaps depicting 2 kb regions (rows) centered at CTCF bound insulator sites that are hypomethylated in HL-60, K562, or both cell lines (as in FIG. 17C). Panels from left to right show methylation calls from 450 k methylation array, reduced representation bisulfite sequencing (RRBS), expanded representation bisulfite sequencing (XRBS), and whole genome bisulfite sequencing (WGBS). All datasets except expanded representation bisulfite sequencing (XRBS) were retrieved from the Encyclopedia of DNA Elements (ENCODE). FIG. 23H is a scatterplot showing merged CTCF binding sites from HL-60 and K562 CTCF ChIP-seq datasets. Individual CTCF binding sites (dots) are colored (i.e., a darker shade of grey) if specifically hypermethylated (darker shade of grey primarily near the y-axis) or hypomethylated (darker shade of grey primarily near the x-axis) in K562 cells.

FIGS. 24A to 24H are plots, bar plots, and heatmaps presenting an evaluation of single cell expanded representation bisulfite sequencing (XRBS) profiles. FIG. 24A is a plot showing unique reads as a function of aligned reads in single cell expanded representation bisulfite sequencing (XRBS) profiles. With greater sequencing depth the fraction of unique reads decreases, as the chance of sampling a non-unique read (i.e. polymerase chain reaction (PCR) duplicate) increases. FIG. 24B presents a bar plot showing the fraction of aligned reads for each single cell library. FIG. 24C presents a bar plot showing the fraction of unique reads (i.e. reads not representing PCR duplicates) per single cell library. Within the same polymerase chain reaction (PCR), the duplicate rate was very similar, irrespective of the total number of aligned reads per single cell. FIG. 24D presents a heatmap comparing alternate allele frequencies from single nucleotide polymorphism (SNP) array data for K562 and HL-60 cell lines. Cell line-specific homozygous alleles are indicated by white boxes and were used for single cell single nucleotide polymorphism (SNP) analysis in FIG. 18D. FIG. 24E provides plots showing copy number variation calls from combined single cell expanded representation bisulfite sequencing (XRBS) profiles (top) and whole exome sequencing data (middle) for K562 cells. A number of chromosomes showed differences in copy number between expanded representation bisulfite sequencing (XRBS) and whole exome sequencing (bottom). However, these differences likely represent true copy number variations between K562 cells used for these experiments. FIG. 24F provides a heatmap showing pairwise correlation coefficients of single cell methylation profiles. The dendrogram shows unsupervised clustering. Single cell expanded representation bisulfite sequencing (XRBS) profiles clustered by cell type. FIG. 24G is a bar plot showing K562 single cell average DNA methylation values within various early and late replicating regions. FIG. 24H presents a heatmap showing pairwise correlation of average DNA methylation values within various early and late replicating regions. Late replicating regions (G2 phase) cluster separately. These results suggest that one source of single cell DNA methylation heterogeneity is decreased fidelity of maintenance DNA methylation in late replicating domains.

### DETAILED DESCRIPTION OF THE INVENTION

As described below, the present invention features compositions and methods for assaying DNA methylation in a single cell.

The invention provides an extended representation bisulfite sequencing method (expanded representation bisulfite sequencing (XRBS)) for targeted profiling of DNA methylation. The method draws a balance between expanding coverage of regulatory elements and enriching for informative CpG dinucleotides in promoters, enhancers, and CTCF insulators. In various aspects, barcoded DNA fragments are pooled prior to bisulfite conversion, allowing multiplex processing and technical consistency in low input samples. The Examples provided herein present the application of expanded representation bisulfite sequencing (XRBS) to leukemia cells to determine genetic copy-number variations and evaluate methylation variability across single cells. Not wishing to be bound by theory, the analysis provided in the Examples demonstrate the utility of the method by demonstrating, through use of data gathered using expanded representation bisulfate sequencing (XRBS), that heterochromatic H3K9me3 regions have the highest cell-to-cell variability in their methylation, likely reflecting inherent epigenetic instability of these late replicating regions, compounded by differences in cell cycle stages among sampled cells.

Not wishing to be bound by theory, DNA methylation of CpG dinucleotides impacts transcriptional activity and genome stability, and is altered in human disease. In various aspects, expanded representation bisulfite sequencing (XRBS) leverages an early barcoding step for high sensitivity and sample multiplexing, and enriches for regions where CpG methylation has been shown to be functionally-relevant-promoters, CpG islands, CTCF insulators, and enhancers. Expanded representation bisulfite sequencing (XRBS) thus provides significant advantages over prior methods in terms of its efficiency, coverage, and sensitivity.

Single cell expanded representation bisulfite sequencing (XRBS) data provides for the resolution of DNA methylation, the linking of heterogeneity to late replicating domains, and the identification of both epimutations and genetic mutations, including copy number variations (CNVs). Expanded representation bisulfite sequencing (XRBS) complements the existing repertoire of single cell epigenetic technologies by providing a highly multiplexed and sensitive method that targets informative genomic regions and is compatible with small inputs and single cells. The computational strategies introduced herein can be used to contextualize single cell DNA methylation within a background of genetic alterations, an area of interest especially in the field of cancer epigenetics. Expanded representation bisulfite sequencing (XRBS) data, as shown in the Examples provided herein, enabled the identification of the highest cell to cell variability in DNA methylation at late replicating H3K9me3-marked regions. This heterogeneity may be a result of the innate variability of these regions, and is likely compounded by variability in cell cycle phase of the individual cells for which single cell methylomes were collected. In other embodiments, XRBS could leverage droplets or nanowells to increase throughput and gain new insights into methylomes and their variability in tissues, tumors, and experimental models.

### DNA Methylation

DNA methylation plays a role in many biological processes, including tumorigenesis. It is hypothesized that rare, tumor promoting or tumorigenic DNA methylation events can be selected for during clonal evolution. Understanding tumor heterogeneity is critical for the development of effective long-term cancer therapeutics. Single cell technologies represent a powerful approach to improving understanding of the transcriptional and epigenetic heterogeneity in tumors.

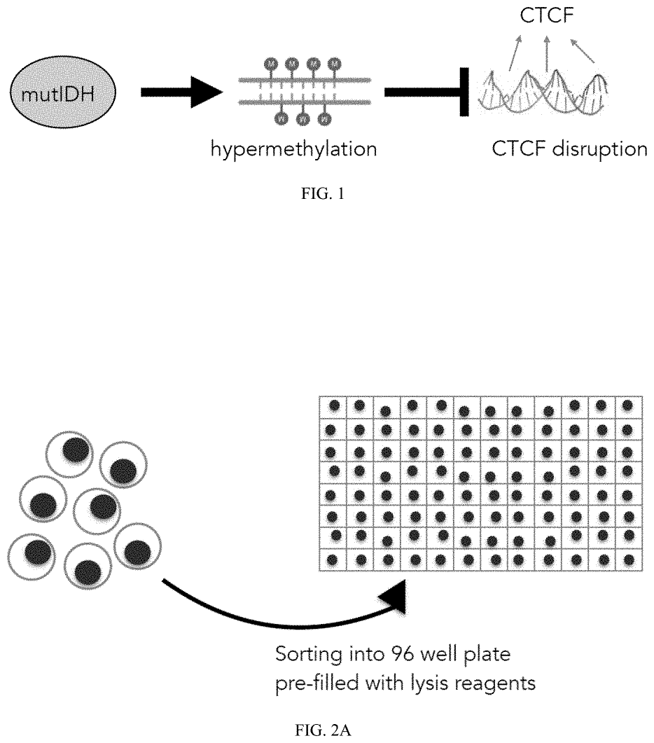

Dynamic DNA methylation contributes to tumor heterogeneity and pathogenesis. For example, isocitrate dehydrogenase (NADP(+)) 1 (IDH) is a metabolic enzyme that is mutated in some cases of acute myeloid leukemia (AML), glioma, and chondrosarcoma. Mutated IDH produces 2-hydroxyglutarate, which inhibits demethylases. This leads to hypermethylation that can, for example, result in disrupted CTCF binding (FIG. 1). CTCF is a DNA binding insulator protein that plays important roles in nuclear architecture, ensuring proper organization of DNA and controlling enhancer-promoter interactions.

Evaluating heterogeneity through single cell technologies has yielded insights into tumor biology. Specifically, single cell RNA sequencing has enabled a deep dive into transcriptional heterogeneity and the multiple cellular identities present in a tumor. Similarly, understanding epigenetic heterogeneity provides insights into regulatory mechanisms. Prior to the present invention, single cell epigenetic technologies read out DNA methylation through bisulfite sequencing and DNA accessibility, an indicator of active regions of the genome, through ATAC-sequencing. While single cell methylation technologies have provided insights into various biological systems, including the human and mouse brain, further development of a single cell methylation technology could make it more accessible for the broader scientific community. As the cost of sequencing decreases, these highly multiplexed methods will increase the number of cells profiled.

The present invention features compositions and methods that are useful for determining the methylation profile of a cell. The method is a low-input, single cell DNA methylation assay that enriches for enhancers. Advantageous features of the method include early barcoding that allows multiplexing, combined digestion and ligation, bisulfite conversion on streptavidin beads, and libraries compatible with Next-Generation Sequencing (e.g., Illumina sequencing platform). The methods of the present invention capture many more enhancers (5× or more) and 40% or more of total cell type agnostic enhancers than conventional methylation assays. Low input methylation profiles generated by the presently described methods are robust, and the quality of the single cell data produced enables clustering by cell identity.

### Low Input Methylation Assay

The disclosure provides a low input (e.g., single cell, 10, 25, 50, 100, 250, 500, 750, 1000 cells) methylation assay that is based on bisulfite chemistry, the standard for distinguishing methylated from unmethylated cytosines. Challenges that arise from the harsh conditions of bisulfite conversion (i.e., loss of nucleic acid) are reduced or eliminated in the present invention by pre- and post-bisulfite tagging to increase the capture of single-stranded converted DNA fragments.

The low input methylation assay enables capture of an informative CpG dinucleotide at the beginning of reads (p7 index), which obviates complicated trimming steps required with standard RRBS protocols, which typically utilize a fill in step and can thereby lose methylation information as well as influence methylation calling. Trimming reads generated from RRBS protocols can result in loss of methylation information and influence methylation calling. The present method also expands coverage of enhancers compared to other methodologies. This is accomplished with hexamer-based second strand synthesis and reduces overall cost of reagents (e.g., hemi-methylated adapters rather than fully methylated adapters). The method also maintains asymmetric adaptation not present in methods like single cell Assay for Transposase Accessible Chromatin (ATAC). Essentially, by tagging nucleic acid fragments with adapters prior to bisulfite conversion, more nucleic acid is retained after conversion compared to other methods. Thus, the present invention provides a significant improvement over known methods and will make single cell DNA methylation profiling more accessible to the broader scientific community.

### Method Overview

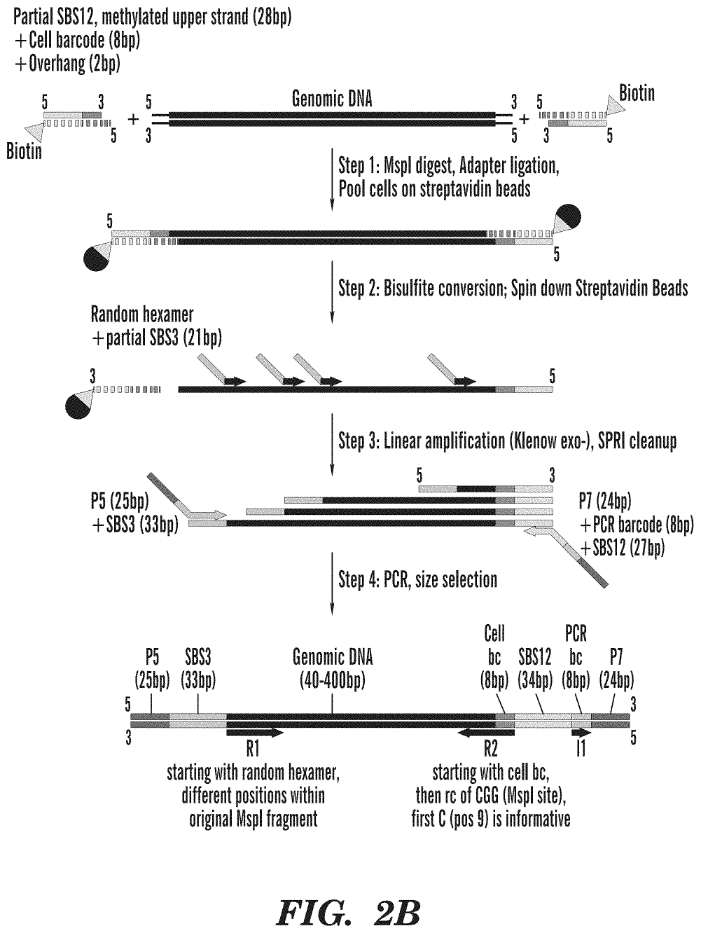

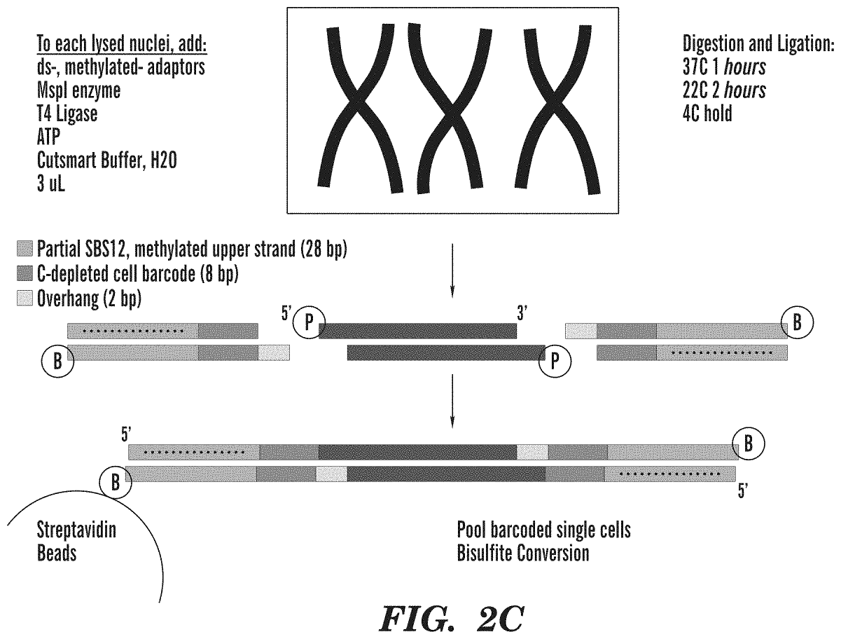

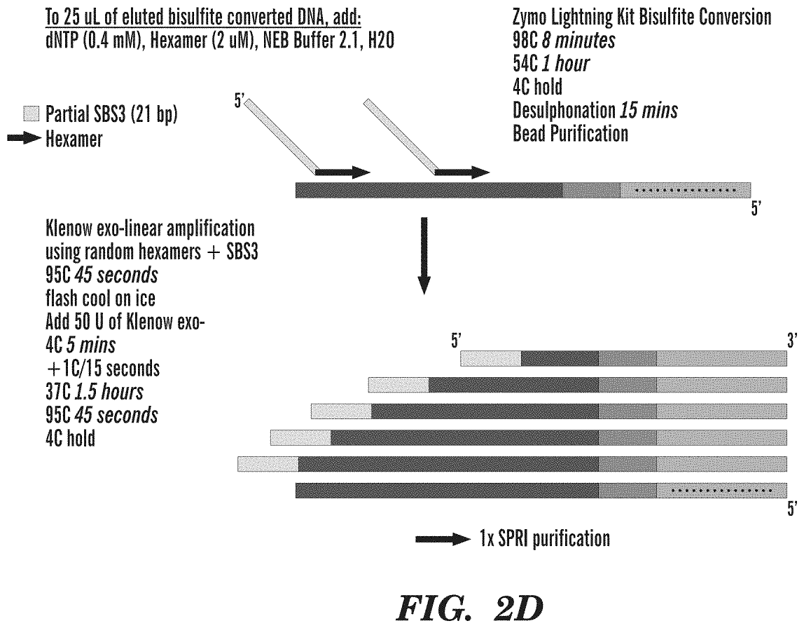

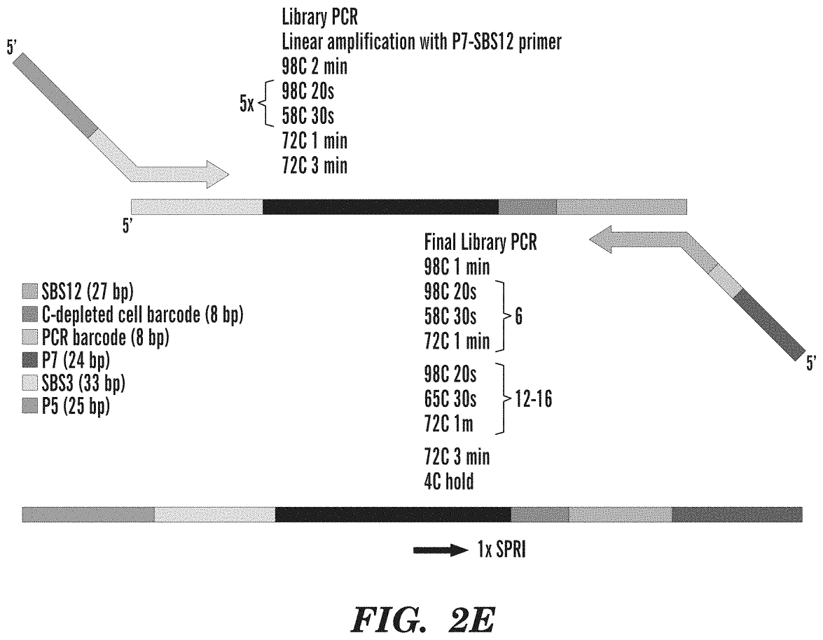

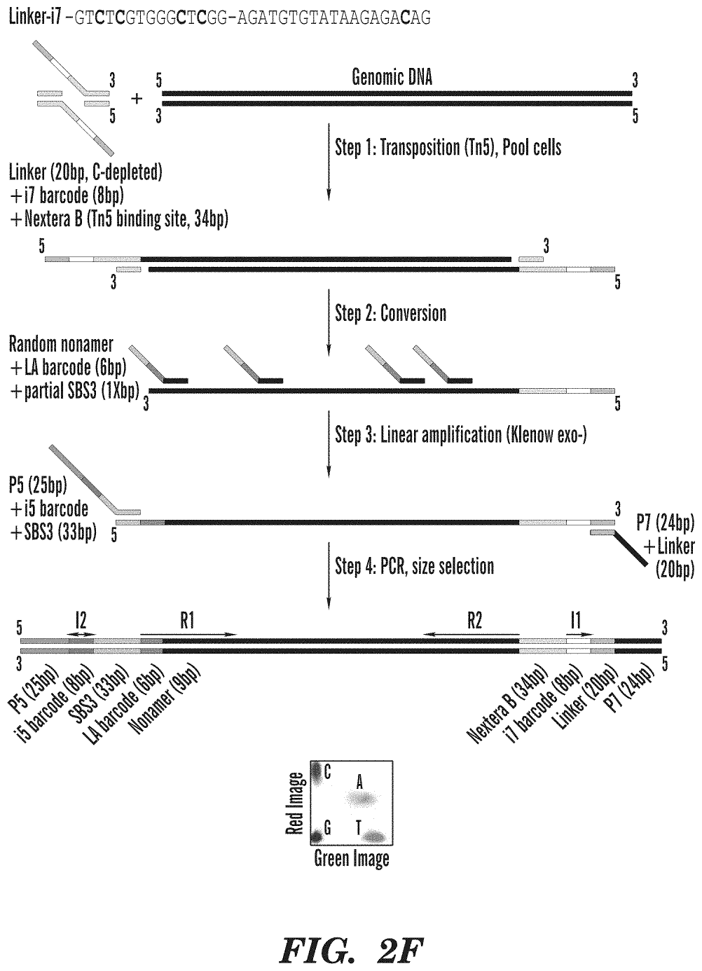

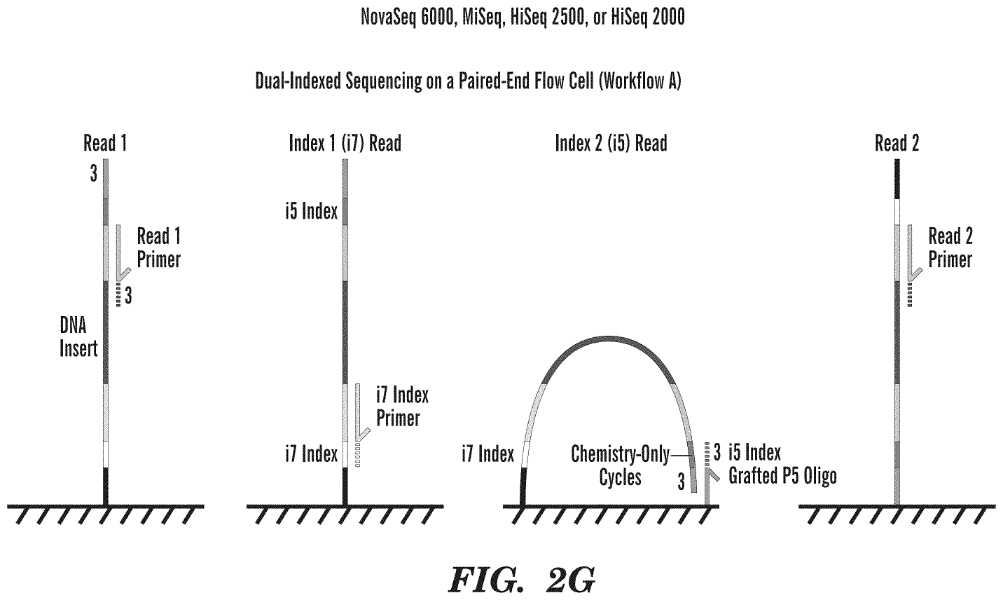

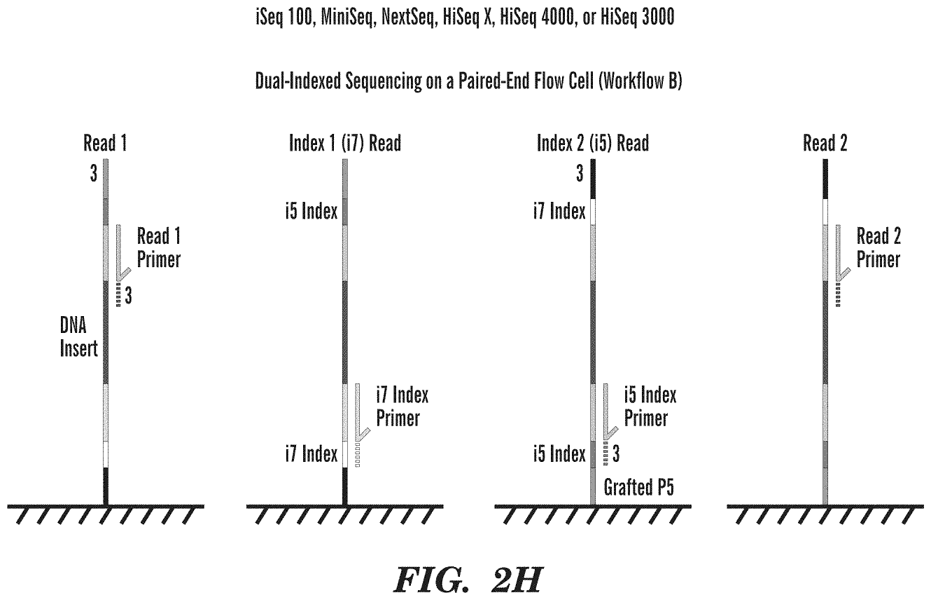

The present invention provides methods of detecting methylation at single cell resolution (FIGS. 2A-2F). In one embodiment of the present invention, viable cells, counterstained with propidium iodide, are sorted via flow cytometry and then lysed. In some embodiments, individual cells are deposited in a well on a 96-well or 384-well plate pre-loaded with lysis reagents. The lysis reaction is incubated (e.g., 75° C.) for a sufficient time to lyse the cells (e.g., 30 minutes). In some embodiments, a protease is added to the lysis reaction and incubated (e.g., for 4 hours) prior to deactivating the reaction. In some embodiments, the lysis reaction is heat deactivated. In some embodiments, such as in bulk or low input applications, the lysed product is purified to retain nucleic acid molecules (e.g., genomic DNA). In some embodiments, the lysed product is not purified after lysis and before digesting and ligation.

The lysis reaction product is then contacted simultaneously with a restriction enzyme, a ligase, and adapter molecules. MspI restriction endonuclease cleaves nucleic acid molecules at CCGG sequences, with the cut being made between the cytosine nucleotides. Adapters are designed such that adapter ligation to digested DNA ends provides resistance to restriction enzyme activity (i.e., sequence recognized by the enzyme is destroyed), whereas a digested nucleic acid that intramolecularly ligates recreates the cut site and is susceptible to digestion again. This property of the adapters pushes the equilibrium away from nucleic acids being digested and then re-ligating intramolecularly and towards intermolecular ligation (i.e., adapter-nucleic acid). It also obviates the typical fill-in method and A tailing used in many library preparation protocols. Because the adapters lack a 5′ phosphate, ligase only creates a covalent bond between the phosphorylated the 5′ end of the fragmented DNA and the 3′ end of adapter (note that the 5′ end of the bottom strand of the adapter being unphosphorylated cannot covalently bond to the 3′ hydroxyl end of the digested nucleic acid).

A biotin label on the 3′ end of the bottom strand of the adapter enables streptavidin bead capture of all adapters that are hydrogen bonded/base paired to the covalently attached top strands. The beads can then be combined from different wells and volume adjusted. Pooled beads bound by biotinylated adapters are resuspended and placed in bisulfite conversion reagent. This involves heat denaturing the DNA double helix, thereby freeing ligated fragments from streptavidin beads. Bisulfite conversion, which involves a heating step that makes double stranded DNA single stranded, obviates any complicated steps in breaking the biotin-streptavidin bond. Elution from the beads is used for the remainder of the bisulfite conversion (Zymo Lightning Kit). In some embodiments that do not require bisulfite sequencing (e.g., nanopore sequencing and other technologies), the nucleic acid molecule is denatured (e.g., heat, chemical, or the like) to generate single stranded nucleic molecules.

Randomly annealing hexamers are used to adapt the 3′ end of DNA fragments and create a complementary second strand for bisulfite converted template, using a strand displacing polymerase (e.g., Klenow exo− or Bst DNA polymerase). A solid phase reversible immobilization (SPRI) cleanup is followed by PCR amplification with P7 and P5 barcodes (e.g., P7 (CAAGCAGAAGACGGCATACGAGAT) and P5 primers (AATGATACGGCGACCACCGA)) and a final library clean up. The generated libraries are compatible with NGS platforms (e.g., Illumina sequencing) and can be spiked into sequencing runs with other libraries. This reduces sequencing associated costs and complexity compared to the sciMET protocol, which requires custom sequencing primers and read lengths.

In one embodiment, MspI digestion and adapter ligation to form adapter-bound DNA fragments is replaced by using a transposase to generate DNA fragments having adapters at their termini. In some embodiments, the transposase is Tn5. This is then followed by bisulfite conversion, Klenow exo− mediated linear amplification, and additional PCR amplification similar to that described above (FIG. 2F). Removal of DNA from chromatin can reduce transposase bias.

### Sequencing

The libraries generated by the present invention can be sequenced using any sequencing platform known in the art. Such technologies at present include, but are not limited to, chain-termination sequencing (Sanger Sequencing), Maxam-Gilbert sequencing, shotgun sequencing, single-molecule real-time sequencing, pyrosequencing, sequencing by synthesis, sequencing by ligation (SOLiD sequencing), nanopore sequencing, and massively parallel signature sequencing. However, it should be understood that any method that is now known or will be developed or otherwise known in the future, is contemplated for use in the present invention. In some embodiments, the sequencing platform is a next generation sequencing (NGS) platform. In some embodiments, the sequencing platform is NovaSeq 6000, MiSeq, HiSeq 2500, or HiSeq 2000. A workflow for these platforms is provided in FIG. 2G. In some embodiments, the sequencing platform is iSeq 100, MiniSeq, NextSeq, HiSeq X, HiSeq 4000, or HiSeq 3000. A workflow for these platforms is provided in FIG. 2H. In some embodiments, the sequencing platform is Illumina NextSeq 500.

### Samples

Assays described herein are used to profile the methylation state of a sample (e.g., biological sample) comprising a polynucleotide (e.g., a polynucleotide derived from a single cell or a population of cells). Likewise, the assays may be used to assess a single cell type or a sample comprising multiple cell types. The cells may be derived from cells cultured in vitro or from cells derived directly from a biological sample, such as a biological sample from a subject to be tested. Samples comprising cells to be assayed using the methods disclosed herein can be obtained from a variety of biological sources (e.g., human or animal sources). In some embodiments, the sample is a tissue sample. In another embodiment, the biologic sample is a biologic fluid sample. Biological fluid samples include blood, blood serum, plasma, saliva, or any other biological fluid useful in the methods of the invention. In some embodiments, the sample is a paraffin embedded sample (e.g., paraffin embedded tissue) or a formalin fixed paraffin embedded tissue sample. These types of samples can suffer from DNA degradation, but are routinely collected in clinical settings. Because the methods robust in the face of degradation of DNA, in some embodiments the sample or nucleic acid molecule inputs are degraded b/c it's robust in the face of degradation of DNA. In some embodiments, intact nuclei can be isolated from fixed tissue (or other samples) for use in the methods described herein. In some embodiments, the sample comprises cell free nucleic acid, such as, but not limited to, cell free tumor nucleic acid and cell free fetal nucleic acid. In some embodiments, the sample comprises tissue biopsies, blood draws, buccal swabs, hair, sweat, skin, semen, and mucus. In some embodiments, the sample comprises cells from a subject, for example, circulating tumor cells, blood cells, or skin cells. In some embodiments, the template nucleic acid molecule is isolated or purified before amplification. Methods of isolating and purifying nucleic acids are well known in the art.

### Adapters, Tags, and Primers

The present invention provides adapters and primers. The barcoded adapters may comprise DNA, RNA, nucleotide analogs or a combination thereof. In one embodiment, an adapter of the present invention is a double-stranded asymmetric adapter molecule comprising a hemi-methylated region (i.e., the top strand is methylated or partially methylated but the bottom strand is not), a C-depleted cell barcode, and an overhang. In some embodiments, the hemi-methylated region comprises a nucleic acid sequence that is used in a next generation sequencing platform (e.g., Illumina). By being hemi-methylated, the adapter is resistant to MspI digestion.

In one embodiment, at least one free end of a cleaved polynucleotide (e.g., DNA) is ligated to a barcoded adapter. The barcoded adapter facilitates further amplification and sequencing of the fragmented polynucleotide (e.g., DNA). The barcode in turn is unique to each individual cell, thereby preserving the origin of the cleaved DNA. All cleaved DNA may be pooled. The presence of the barcode allows the DNA to be mapped back to a particular cell or cell population.

The C-depleted barcode allows tracking of sequencing data generated from an individual cell. In some embodiments, the C-depleted barcode is between 5 and 25 bp. For example, the C-depleted barcode can be 5, 6, 7, 8, 9, 10, 11, 12, 13, 14, 15, 16, 17, 18, 19, 20, 21, 22, 23, 24, or 25 bp. In some embodiments, the C-depleted barcode is less than 5 bp. In some embodiments, the C-depleted barcode is greater than 25 bp.

The overhang present in the adapter molecule allows the adapter molecule to hybridize with digested DNA. In some embodiments, the overhang comprises between 1-5 nucleotides.

In some embodiments, the adapter molecules of the present invention comprise at least a partial sequencing primer binding site. In some embodiments, the partial sequencing primer binding site is proximate to a barcode sequence. In certain example embodiments, a spacer of 1 to 20 nucleotides in length may be located between the sequencing primer binding site and the barcode. In certain example embodiments, the spacer may function as a unique molecular identifier (UMI). The UMI enables further differentiation between any two distinct adapter ligation events that may occur at a cleavage site in the DNA from two different cells. In other example embodiments, the first portion of the second read sequencing primer binding site may be located directly adjacent to the barcode sequence. The barcode is a short sequence of nucleotides (e.g. DNA, RNA, or nucleotide analog), for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides. In certain example embodiments, the barcode is eight nucleotides in length. In one embodiment, the sequence is present at the terminus being ligated to digested DNA.

In some embodiments, the hemi-methylated double-stranded adapter comprises a label on the 3′ end of the non-methylated strand. For example, in some embodiments, the adapter has a label or a first member of a binding pair that can interact with another molecule (e.g., a capture molecule or a second member of a binding pair). In some embodiments, the first member of a binding pair A nonlimiting example of a label or a first member of a binding pair that can be used in the present invention is biotin. Because of its affinity for streptavidin, nucleic acid molecules comprising biotin labeled adapters can be bound to streptavidin (see FIG. 2B). In some embodiments, the streptavidin is bound or conjugated to a substrate. In some embodiments, the substrate is a bead, a membrane, a chip, and a slide.

In some embodiments, post-bisulfite conversion amplification reactions can be primed with nucleic acid molecules comprising a sequence complementary to a sequence of the bisulfite converted DNA and a tag sequence. In some embodiments, the tag sequence can be used as a binding site for subsequent amplification or sequencing primers. In some embodiments, the tag comprises a partial SBS3 nucleic acid sequence. In some embodiments, the tag sequence comprises between 10 and 25 bp. In some embodiments, the tag sequence is 10, 11, 12, 13, 14, 15, 16, 17, 18, 19, 20, 21, 22, 23, 24, or 25 bp. In some embodiments, the tag sequence is less than 10 bp. In some embodiments, the tag sequence is greater than 25 bp.

In certain example embodiments, the adapter may comprise one or more modifications that increase adapter stability and/or resistance to degradation. In certain example embodiments, the modifications may be made to one or more terminal bases of the adapter on the 3′ end, the 5′ end, or both. In one example embodiments, the one or more modifications may be made to the first, second, third, and/or fourth terminal bases of the adapter on the 3′ or 5′ end of the sense or antisense strand. In certain example embodiments, the one or more modifications are made to the backbone linkages between the first and second terminal bases; the first, second, and third terminal bases; the first, second, third, and fourth terminal bases; or the first, second, third, fourth, and fifth terminal bases of the adapter on the 3′ or 5′ end of the sense or antisense strand of the adapter. In certain example embodiments, the one or more modifications comprise phosphorothioate linkages between the terminal bases on the 3′ or 5′ end of the sense or antisense strand. In one example embodiment, the one or more modifications comprise phosphorothioate linkages between the first and second terminal bases; the first, second, and third terminal bases; the first, second, third, and fourth terminal bases; or the first, second, third, fourth, and fifth terminal bases of the adapter on the 3′ or 5′ end of the sense or antisense strand. In one example embodiment, the one or more modifications comprise phosphorothioate linkages between the first and second terminal bases; the first, second, and third terminal bases; the first, second, third, and fourth terminal bases; or the first, second, third, fourth, and fifth terminal bases of the adapter on the 3′ end of the sense strand. In certain example embodiments, the modifications described in the immediately preceding sentence are further combined with phosphorothioate linkages between the first and second terminal bases; the first, second, and third terminal bases; the first, second, third, and fourth terminal bases; or the first, second, third, fourth, and fifth terminal bases of the adapter on the 5′ end of the antisense strand.

The amplification promoter is located proximate to at least the partial sequencing primer binding site. As used herein, “sequencing primer binding site” refers to a region comprising a nucleotide sequence complementary to a nucleotide sequence of a sequencing primer and to which the sequencing primer can hybridize to initiate a sequencing read. Thus, the nucleotide sequence of the sequencing primer used will dictate the sequence of the partial sequencing primer binding site on the adapter. In certain example embodiments, a spacer of 1 to 8 nucleotides in length may be located between the amplification promoter and the partial sequencing primer binding site. In certain other example embodiments, the amplification promoter and the partial sequencing primer binding site are directly adjacent to one another. In certain example embodiments, the partial sequencing primer binding site comprises 10, 11, 12, 13, 14, 15, 16, 17, 18, 19, 20, 21, 22, 23, 24 or 25 nucleotides complementary to a sequencing primer. In certain example embodiments, the partial sequencing primer binding site is a first read sequencing primer binding site. In certain example embodiments, the first read sequencing primer binding site is a SBS3 primer binding site. In certain other example embodiments, the sequencing primer binding site is a second read sequencing primer binding site. In certain example embodiments, the second read sequencing primer binding site is a SBS12 sequencing primer binding site. The SBS3 and SBS12 sequencing primers are provided as example only and binding sites based on other suitable sequencing primers may be used.

In some embodiments, libraries of amplified nucleic acid sequences are generated. The amplified DNA is sequenced using a suitable sequencing technology, such as a next generation sequencing method. The resulting sequencing data is then demultiplexed based on the one or more barcodes. For example, if an assay specific barcode is incorporated as described above, the sequence data may be demultiplexed based on the assay barcode such that all sequences detected as having a particular nucleosomal DNA modification are grouped first. The sequencing data may then be further grouped according to the origin specific barcode to identify all nucleosomal DNAs having a particular nucleosomal modification and originating from the same cell or cell population and any perturbations that may have been applied to the cell or cell population prior to conducting the assay.

Such library creation can require additional primers. For example, a library primer can comprise a nucleic acid sequence that is complementary to the tag sequence or the hemi-methylated sequence discussed above. In some embodiments, a forward library primer and a reverse library primer bind to the tag sequence and the hemi-methylated sequence, respectively, or vice versa. In some embodiments, the library primer can comprise a PCR barcode, a P7 or a P5 nucleic acid sequence, or combination thereof. The PCR barcode can reside on a library primer that also has the P7 or P5 nucleic acid. The PCR barcode, in some embodiments, resides 3′ to the P5 or P7 nucleic acid sequence.

In some embodiments where a transposase is used to break up genomic DNA and attach asymmetric adapters to the DNA, the adapter comprises a 20 bp cytosine-depleted linker. In some embodiments, the adapter comprises a 34 bp Nextera B sequence having a Tn5 binding site. In some embodiments, a Nextera B sequence is GTCTCGTGGGCTCGGAGATGTGTATAAGAGACAG.

The adapters and primers described herein can be used on next-generation sequencing platforms. Additionally, substituting primers used in one platform for primers used in another platform is well within the reach of one skilled in the art.

### Single Cell Technology for Enhancer Methylation Profiling

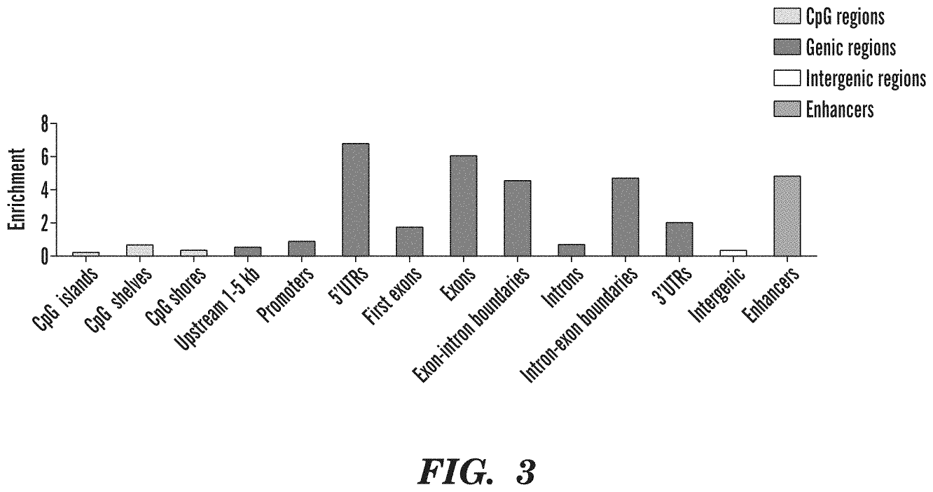

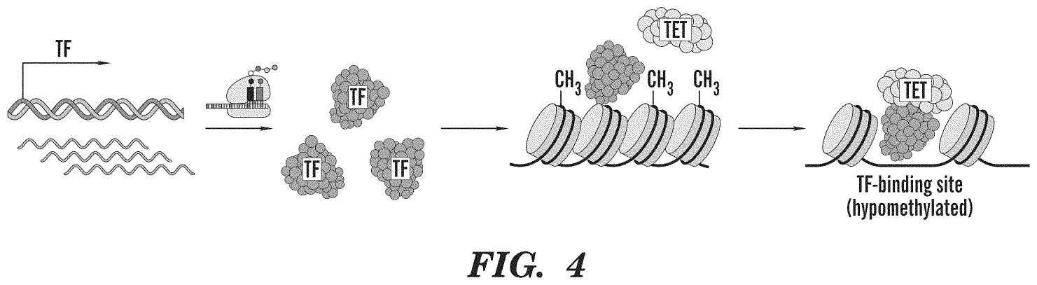

The present invention provides methods for detecting differentially methylated regions in a genome. Differentially methylated regions between hepatocellular carcinomas and matched normal cells occur in promoters and enhancers (FIG. 3). Additionally, aberrant enhancer hypomethylation has been shown to contribute to hepatic carcinogenesis through global transcriptional reprogramming. Referring to FIG. 4, enhancer hypomethylation can play a significant role in gene activation by relaxing chromatin structure and providing greater access to the DNA for transcription factors to bind.

The methods of the present invention are uniquely suited to identify and characterize enhancer methylation. The protocol combines pre-bisulfate adapter tagging with a hexamer based amplification that allows multiplexing. In some embodiments, the cells are sorted into reaction vessels. For example, in some embodiments, a reaction vessel is a well, droplet, tube, or microfluidic chamber. In some embodiments, cells are first sorted into lysis reactions (such as a 96-well plate pre-filled with lysis reagents (FIG. 2A)).

In some embodiments, double stranded, methylated adapters along with the MspI restriction enzyme, and a ligase (e.g., T4 ligase) are added to the lysed cells. In some embodiments, the ligation and the digestion reactions occur simultaneously (FIG. 2C).

The resulting barcoded nucleic acids derived from single cells can be pooled. In some embodiments, one strand of the adapter molecule will be labeled such that it and its associated nucleic acid can be immobilized. In some embodiments, the adapter molecule will have a biotin label, which will bind to streptavidin beads. The streptavidin-bound nucleic acids can then be subjected to bisulfite conversion (e.g., using the Zymo Lightning Kit) (FIG. 2D). The resulting bisulfite converted nucleic acids can then be amplified using, for example, a Klenow exo− linear amplification protocol. Because the sequence of the genomic DNA is not known, the amplification reaction can be primed with random hexamers that have a 5′ tag (e.g., a partial SBS3 nucleic acid sequence) (FIG. 2D).

The amplified nucleic acid molecules can be purified (e.g., solid phase reversible immobilization (SPRI) purified, such as with biotin and streptavidin). The purified nucleic acid molecules can then be further amplified to generate a library. In some embodiments, more than one amplification reaction is used to prepare the library (FIG. 2E).

In another aspect of the present invention, a method is provided for detecting methylation in a cell, wherein the genomic DNA is fragments by a transposase enzyme inserting an adapter molecule (FIG. 2F). In some embodiments, after transposase treatment, the adapter-labeled DNA fragments are pooled and subjected to bisulfite conversion and Klenow exo− mediated linear amplification. The primers used for the linear amplification include, in some embodiments, a barcode, a sequence used in an NGS platform, and a random hexamer or nonamer. The linear amplification products are then PCR amplified prior to library formation and/or sequencing (FIG. 2G, 2H).

The present invention is well-suited for droplet based chemistry. Using droplet based chemistry can reduce costs and allow integration of the method into microfluidic platforms.

### Diagnostic Assays

The present invention provides methods and compositions for detecting methylation profiles of cells that are correlated with a disease and can be used to identify subjects with high probability of having or developing the disease. Detection of an alteration relative to a normal, reference sample can be used as a diagnostic indicator of a disease (e.g., cancer). In some embodiments, altered methylation of a particular gene is correlated with a particular disease. In some embodiments, the disease detected by the assay is a cancer. In some embodiments, the cancer is acute myeloid leukemia, glioma, or a chondrosarcoma. In some embodiments, the disease is Lynch Syndrome, an inherited disease resulting primarily in colorectal cancer. In some embodiments, the disease is age related clonal hematopoiesis (ARCH; also called clonal hematopoiesis of indeterminate potential (CHIP)), which is characterized by the gradual clonal expansion of hematopoietic stem and progenitor cells (HSPC) carrying recurrent disruptive genetic variants in individuals without a diagnosis of hematologic malignancy. This is usually diagnosed with sequencing of mutational rate, but could potentially benefit from methyl information of rare populations. In some embodiments the disease is an imprinting disease (e.g., Angelman syndrome (AS), Prader-Willi syndrome (PWS), and the like). Currently, imprinting diseases are diagnosed based on observable symptoms or by sequencing loci associated with a suspected disease. The present invention provides an additional means for diagnosing (or confirming a diagnosis) of an imprinting disease.

The present invention features diagnostic assays for the detection of a disease or the propensity to develop such a condition. In one embodiment, the level of methylation is measured on at least two separate occasions and an increase in the level is an indication of disease progression or regression. In some instances, the level of demethylation increases or the level of methylation decreases over time. The level of methylation in a cell of a subject having a disease or condition or susceptible to develop the disease or condition may be altered by as little as 10%, 20%, 30%, or 40%, or by as much as 50%, 60%, 70%, 80%, or 90% or more relative to the level of methylation in a normal control.

The diagnostic methods described herein can be used to provide a diagnosis individually or to confirm the results of another diagnostic method. Additionally, the methods described herein can be used with any other diagnostic method described herein for a more accurate diagnosis of the presence or severity of a disease.

A methylation profile may be obtained from a subject sample and compared to a reference profile obtained from a reference population, enabling classifying the subject as belonging to or not belonging to the reference population. The correlation of a methylation profile to a disease diagnosis may consider the presence or absence of methylation in test and control samples. The correlation may consider both factors when making a disease status determination.

The invention also provides for methods where methylation profiles are measured before and after subject management. In these cases, the methods are used to monitor the status of the cancer, e.g., a response to treatment, regression, remission, or progression of the disease.

The methylation profiles generated using the methods of the present invention have uses other than just diagnostic. In some embodiments, they can be used in monitoring responses to therapy. In another embodiment, the profiles can be used to study the regulatory regions of a gene associated with a disease. In some embodiments, the methylation profiles generated by the methods disclosed herein are useful in determining the status or stage of a subject's disease. A methylation profile generated for a subject sample using the methods described herein is compared with the methylation profile of a control sample, wherein differences in the levels or amounts of methylation distinguishes disease status from disease-free status. The techniques can be adjusted, as is well understood in the art, to increase the sensitivity or specificity of the diagnostic assay.

While methylation of a particular region or gene in the genome can be a useful diagnostic, in some instances, a combination of methylated genes or regions provides greater predictive value than a methylation profile of a single gene or region. Detection of the presence or absence of methylation at a plurality of genes or regions in a sample can decrease false positives and false negative diagnoses, while increasing the occurrence of true positives and true negatives.

### Kits and Compositions for Detecting and Characterizing Methylation

In another embodiment, kits and compositions are provided that advantageously allow for the detection of methylation in a subject sample (e.g., blood, biopsy, urine, or saliva). In one embodiment, the kit includes a composition comprising reagents for performing an amplification reaction and/or a bisulfate conversion, including adapters as described herein. In some embodiments, the reagents include hemi-methylated adapters, a buffer, MspI or other methylation insensitive restriction enzyme that cuts at cytosines, and/or a polymerase. A non-exhaustive list of methylation insensitive restriction enzyme includes, but is not limited to, MspI, ScaI, BamHI, HindIII, NotI, and SpeI. In some embodiments, the kit comprises a sterile container which contains the amplification reaction reagents; such containers can be boxes, ampoules, bottles, vials, tubes, bags, pouches, blister-packs, or other suitable container forms known in the art. Such containers can be made of plastic, glass, laminated paper, metal foil, or other materials suitable for holding amplification reagents.

In another embodiment, the kit includes a composition comprising reagents for performing a sequencing reaction, including nucleic molecules that can specifically bind to an adapter as described above. The reagents, in some embodiments, include nucleotides, labeled nucleotides, a buffer, and any other reagent necessary for performing a next-generation sequencing reaction (e.g., on the Illumina platform). In some embodiments, the kit comprises a sterile container which contains the amplification reaction reagents; such containers are described above. In some embodiments, the kit comprises compositions for amplification and sequencing as described above. Kits may also include instructions for performing the reactions.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

CpG methylation contributes to the stability and transcriptional regulation of mammalian genomes. CpG density is bimodal, depleted from most of the genome, with the exception of high density CpG islands that are frequently promoter-associated. DNA methylation at promoter-associated CpG islands is associated with gene silencing and intricately regulated to guide complex biological processes such as embryogenesis, aging, and tumorigenesis. Beyond promoters, DNA methylation patterns at genomic regulatory elements have been implicated in determining cell identity and chromatin structure, especially at enhancers and CTCF regions. Comprehensive profiling of DNA methylation is important to understand cell state dynamics.

The gold standard method for reading CpG methylation is to convert unmethylated cytosines to uracil with bisulfite treatment prior to sequencing. Whole genome bisulfite sequencing (WGBS) provides coverage of the entire genome, but is inefficient because vast CpG-depleted regions consume sequencing capacity. In contrast, reduced representation bisulfite sequencing (RRBS) uses restriction digest to enrich for CpG dense regions and thus provides high coverage of a fraction of the genome at reduced sequencing cost. However, RRBS lacks coverage of enhancer regions and CTCF binding sites that are outside of CpG islands. Methylation arrays that capture established regions of interest, typically promoters and CpG islands, may not be ideal for profiling regulatory elements, which are of high interest. The disclosure presents a novel strategy for profiling DNA methylation across promoters, enhancers and CTCF sites that is efficient and compatible with low input samples and single cells.

### Example 1

### Enhancer Enriching Methylation Assay

An enhancer enriching methylation assay that allowed for enhancer methylation profiling at single cell resolution is described. Viable cells, counterstained with propidium iodide, were sorted into wells of a 96 well plate preloaded with lysis buffer (FIG. 2A). After incubation for 30 minutes at 75° C., proteinase was added to each well and incubated for 4 hours before heat inactivation. Both digestion of the DNA with MspI and ligation to double stranded adapters occurred concurrently. Adapter sequences were designed such that adapter ligation to digested DNA ends provides resistance to restriction enzyme activity. This enables the equilibrium of MspI cut sites being cut and then re-ligating intramolecularly to be pushed towards adapter ligation intermolecularly. Because the adapters lack a 5′ phosphate, ligase created only a covalent bond at the 5′ end of the fragmented DNA and not the 3′ end.

A biotin label on the 3′ end of the bottom strand of the adapter enabled streptavidin bead capture of all adapters and covalently attached top strands. The beads were then combined from different wells and volume adjusted. Pooled beads bound by biotinylated adapters were resuspended and placed in bisulfite conversion reagent. This involved heat denaturing the DNA double helix, thereby freeing ligated fragments from streptavidin beads. Elution from the beads was used for the remainder of the bisulfite conversion (Zymo Lightning Kit).

Randomly annealing hexamers were used to adapt the 3′ end of DNA fragments and create a complementary second strand for bisulfite converted template, using the Klenow enzyme. A solid phase reversible immobilization (SPRI) cleanup was followed by PCR amplification with P7 and P5 barcodes (e.g., P7 (CAAGCAGAAGACGGCATACGAGAT) and P5 (AATGATACGGCGACCACCGA)) and a final library clean up. The generated libraries were compatible with NGS platforms (e.g., Illumina sequencing) and could be spiked into sequencing runs with other libraries. This reduces sequencing associated costs and complexity compared to the sciMET protocol, which requires custom sequencing primers and read lengths.

More specifically, the assay combined adapter tagging prior to bisulfite conversion with hexamer-based amplification that allows multiplexing. The adapter comprised an asymmetrical double-stranded DNA molecule comprising a partial SBS12 sequence, methylated upper strand (28 bp) and unmethylated lower strand, an 8 bp cell barcode, and a 2 bp overhang extending from the 5′ end of the lower strand. The 3′ end of the lower strand was biotinylated (FIG. 2B).

Genomic DNA was digested with MspI in the presence of the adapter and T4 ligase, ATP, and Cutsmart buffer in a 3 μL reaction volume on a 96-well plate (FIG. 2C). The reaction was incubated at 37° C. for 1 hour and 20° C. for 2 hours. Barcoded DNA from single cells was pooled in the presence of streptavidin beads and subjected to bisulfite conversion (Zymo Lighting Kit). For bisulfite conversion, pooled DNA was incubated at 98° C. for 8 minutes followed by 54° C. for 1 hour, desulphonated for 15 minutes, and purified by solid phase reversible immobilization (SPRI) according to manufacturer's protocol.

25 μL of eluted bisulfite converted DNA was included in a linear amplification reaction comprising 0.4 mM dNTPs, 2 μM hexamer fused to a partial SBS3 sequencing primer, and NEB Buffer 2.1. The reaction was heated to 95° C. for 45 seconds and flash cooled on ice. 50 units of Klenow exo− was added to the reaction and incubated at 4° C. for 5 minutes followed by increasing the reaction temperature 1° C. every 15 seconds until the temperature was 37° C. The reaction was incubated at 37° C. for 1.5 hours and heat deactivated at 95° C. for 45 seconds (FIG. 2D). The resulting amplification products were SPRI purified.

Referring to FIG. 2E, an initial library PCR reaction was carried out, using the Klenow exo− amplification products as initial templates. Briefly, P7-SBS12 and P5-SBS3 primers were used to prime amplification, and the reaction was carried out under the following conditions: 98° C.—2 minutes; 5 cycles of 98° C.—20 seconds, 58° C.—30 seconds, and 72° C.—1 minute; followed by 3 additional minutes at 72° C. A final PCR reaction was carried out using the following conditions: 98° C.—1 minute followed by six cycles of 98° C.—20 seconds, 58° C.—30 seconds, and 72° C.—1 minute, followed by 12 to 16 cycles of 98° C.—20 seconds, 65° C.—30 seconds, and 72° C.—1 minute, followed by 72° C.—for 3 minutes. The reactions were SPRI purified, and the purified products were sequenced on an Illumina platform.

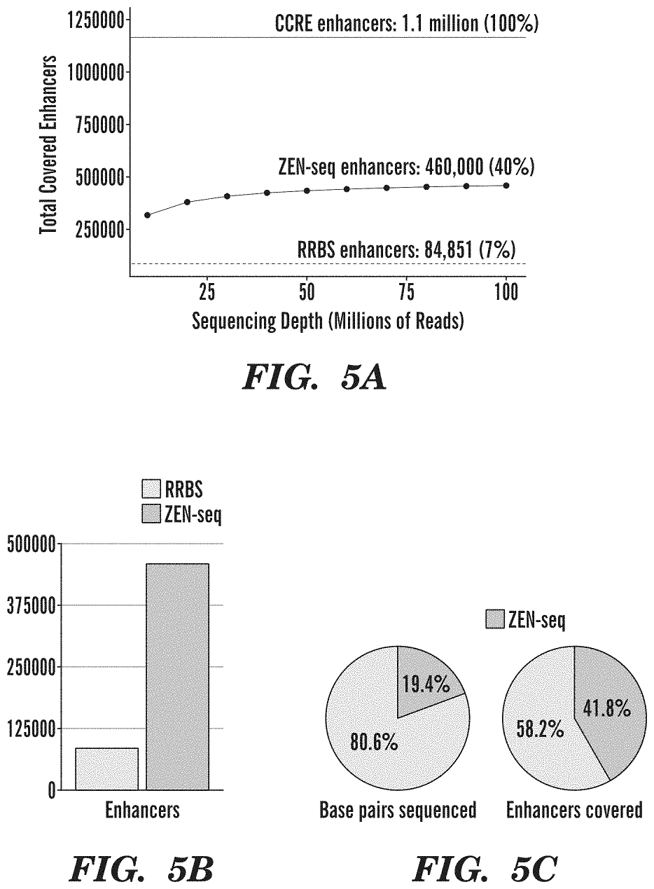

This method captured more enhancers than reduced-representation bisulfite sequencing (RRBS). Referring to FIGS. 5A and 5B, of the possible 1.1 million candidate cis-regulatory elements (CCREs), the presently described method captured 460,000 (41.8%) enhancers compared to only 85,000 (7%) captured by RRBS. Only 581 million base pairs, or approximately 19.4% of the 3 billion base pair genome, were sequenced to capture the 41.8% of enhancers (FIG. 5C)

### Example 2

### Low Input Assay

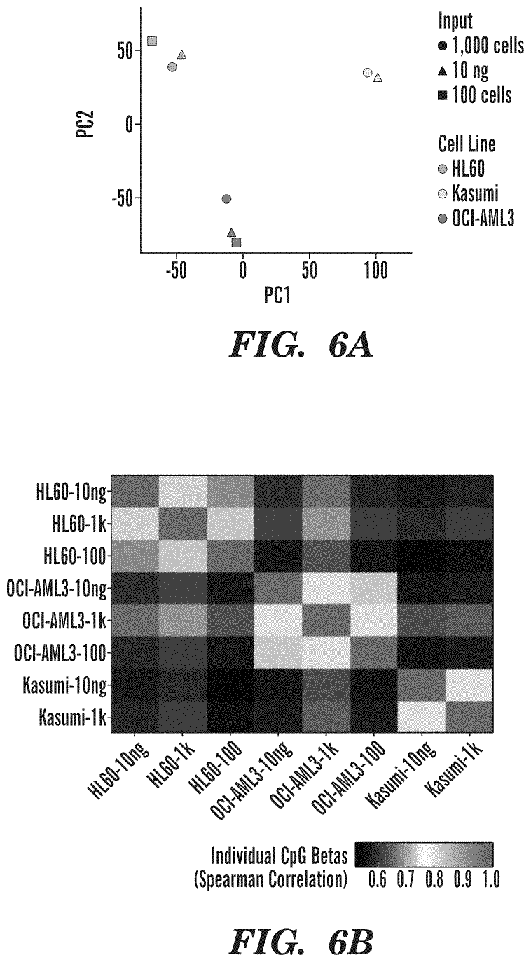

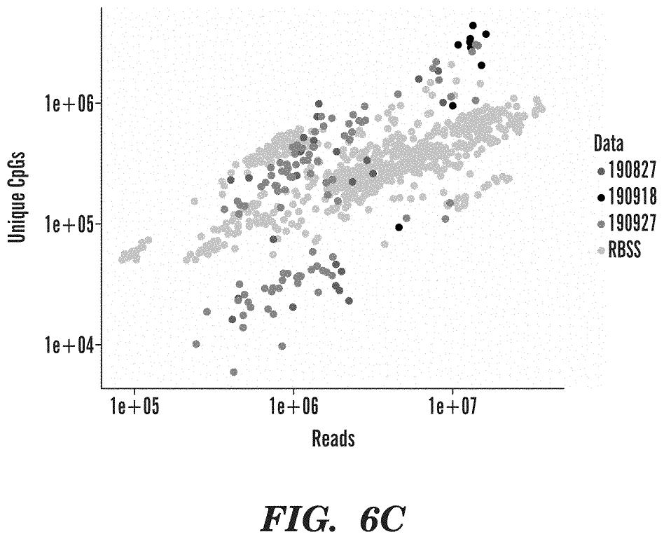

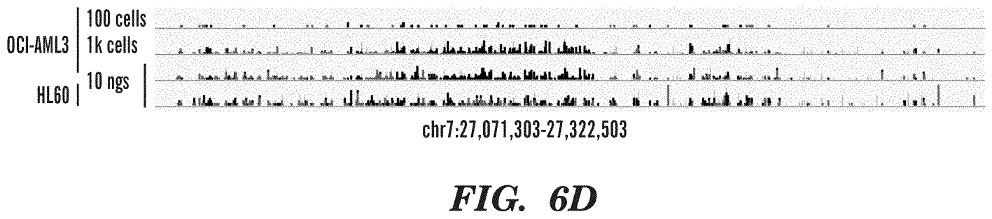

To determine if the assay was sensitive to low levels of starting materials, three different amounts (1,000 cells, 10 ng, and 100 cells) of three different cell lines (HL60, Kasumi, and OCI-AML3) were used as starting materials for the methylation assay. The assay was performed according to the protocol described in Example 1. Principal Component Analysis (PCA) of the sequencing data (FIG. 6D) generated in the assay showed that the method is robust for low inputs. The PCA results showed tight clustering of each cell type regardless of the amount of starting material (FIGS. 6A and 6B). Furthermore, the single cell data quality observed is comparable to that seen in other single cell methods. Specifically, the number of unique CpGs detected relative to the number of sequencing reads generated was similar between the presently described method and other methods known in the art. (FIG. 6C).

### Example 3

### Absence of Barcode Cross Talk

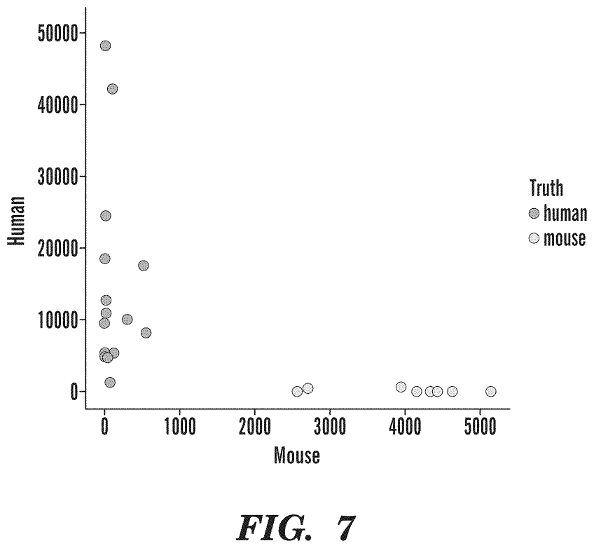

Barcodes are used in the methods of the present invention to identify the source of each sequencing read. This allows one to distinguish between different samples, such as cells derived from different sources. To confirm that the barcodes identify the proper source, human and mouse cells were subjected to the assay described in Example 1, and the cells were mixed. The adapters used with the human cells had different barcodes than those used with mouse cells. The number of mouse and human reads sequenced from each barcode were plotted. Referring to FIG. 7, the barcodes were distributed either with high mouse and low human reads or high human and low mouse reads. Thus, the barcodes effectively distinguished sequencing reads derived from human cells from those derived from mouse cells. The absence of barcode cross-talk indicates that the barcodes are not being contaminated (i.e., each barcode is representing a single cell).

### Example 4

### Single Cell Methylation Profiles Cluster by Cell Identity

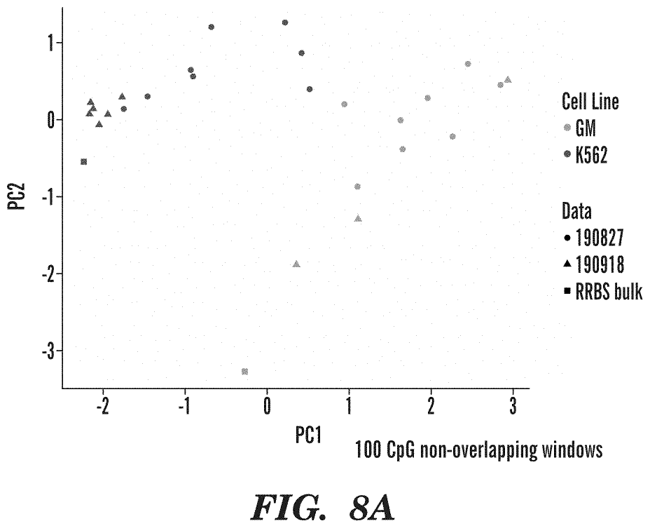

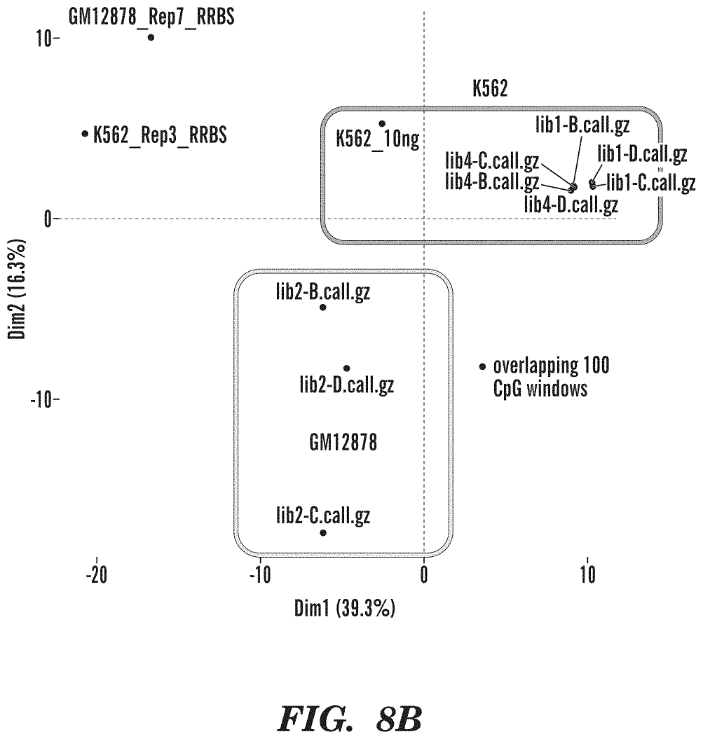

Methylation was assessed in two human cell lines, GM12878 and K562 using the assay described in Example 1. 100 CpG non-overlapping windows were used to generate methylation profiles for the cell lines. Two independent data sets generated from the assay were assessed along with RRBS bulk data using Principal Component Analysis. The methylation profiles for GM12878 cells cluster together with the methylation profiles for the K562 cell (FIGS. 8A and 8B).

### Example 5

### Profiling Methylation in Normal Bone Marrow and in AML Cell Lines

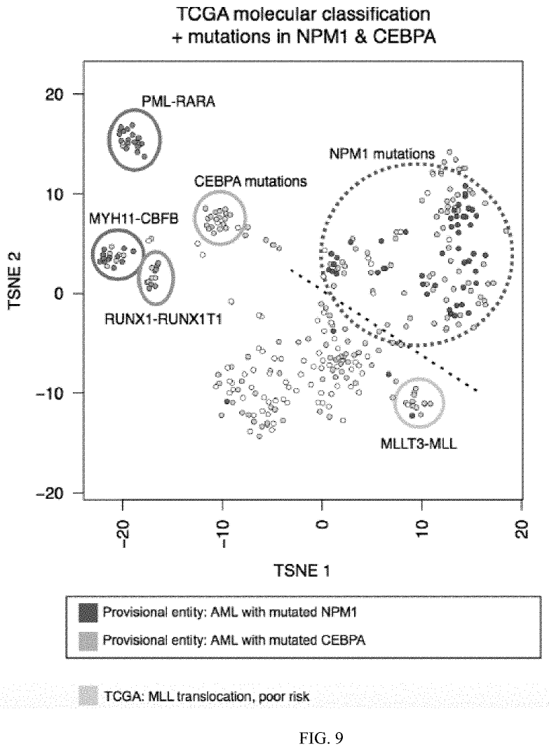

To further validate the methylation assay of Example 1, methylation profiles were generated for normal bone marrow cells lines and AML cell lines. These cell lines were chosen based on tumor methylation analyses. The AML cell lines included OCI-AML3, which harbor NPM1 and DNMT3A mutations and wild type FLT3; HL60, which is hypotetraploid, and Kasumi, which exhibits CEPBA inactivation and RUNX1-RUNX1T1 translocation. As shown in FIG. 9, The Cancer Genome Atlas (TGCA) molecular classification clusters like cells together, allowing one skilled in the art to identify specific cell types. Clustering of methylation profiling of TCGA tumors indicates that genotype of tumors drives distinct methyl signatures. This clustering was used to select representative AML cell lines that could be expected to have different methylation profiles based on their different genotypes.

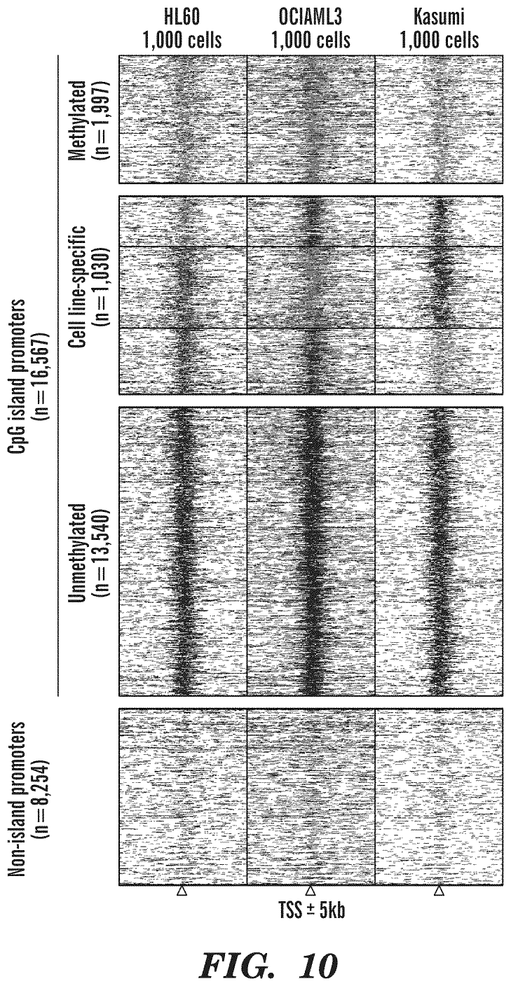

The assay described in Example 1 was used to identify differential promoter methylation in these cell lines. 1030 promoters comprising CpG islands were identified that had cell line specific methylation profiles. Over 8,000 non-CpG island promoters, about 13,500 unmethylated CpG island promoters, and about 2,000 methylated CpG island promoters did not exhibit differential promoter methylation (FIG. 10).

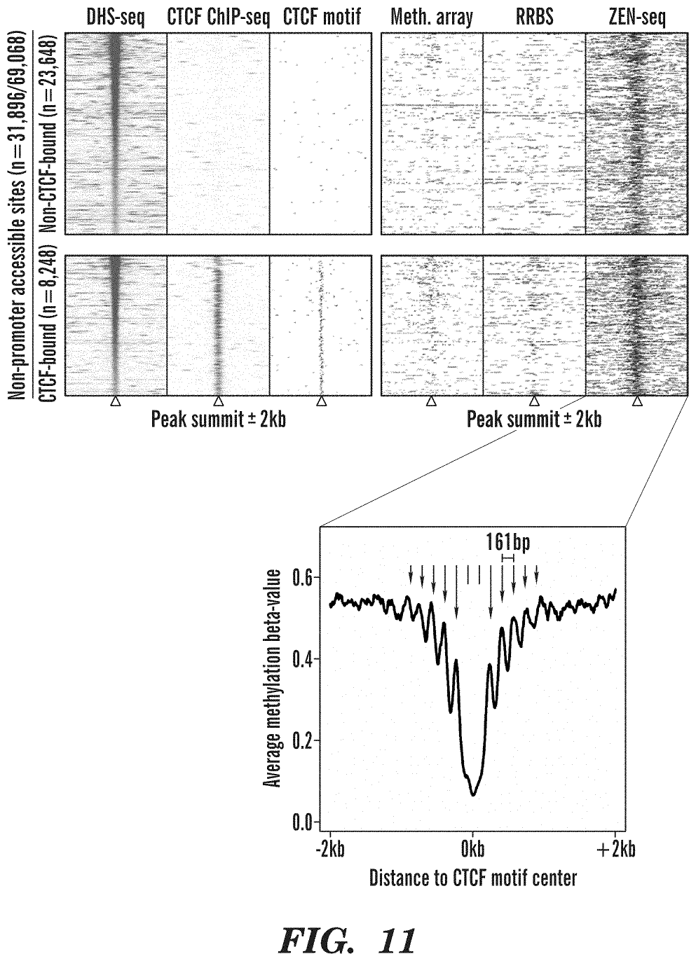

The assay exhibited higher coverage over accessible regions compared to other methodologies (e.g., DNase I hypersensitivity sequencing (DHS-seq), CTCF chromatin immunoprecipitation sequencing (ChIP-seq), CTCF motif, Illumina 450K Methylation Array, and RRBS) (FIG. 11). The assay also detected hypomethylation of a CTCF binding motif and nucleosome periodicity (FIG. 11). The data indicate CTCF methylation status can be better evaluated using the method of Example 1 than either RRBS or methylation array data.

### Example 6

### Characterization of Human Bone Marrow Methylation in Low Input Methylation Assay (100 Cells)

Individual cell methylation profiles may be distinct from a bulk methylation profile of a population of cells. For example, blood or bone marrow, as well as other bodily fluids and tissues, may comprise many different cell types (e.g., B cells, T cells, and the like). Additionally, single cells of the same type (e.g., B cells) may have different methylation profiles. For example, a cell having a mutation in IDH (a demethylase inhibitor), may have a different methylation profile than other cells of the same type that have wild type IDH. Thus, the present invention allows interrogation of single cells as well as interrogation of populations of cells.

The assay was used to generate bulk methylation profiles of bone barrow cells and sorted cell types (e.g., hematopoietic stem cell (HSC)/progenitor cells (CD34+), monocytes (CD14+), and T cells (CD3+)).

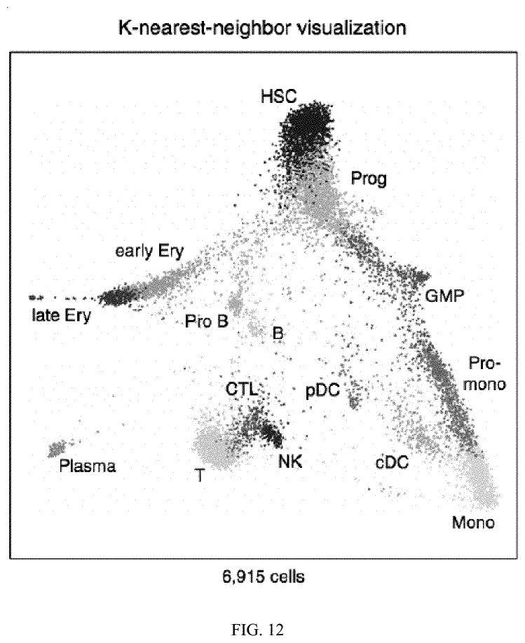

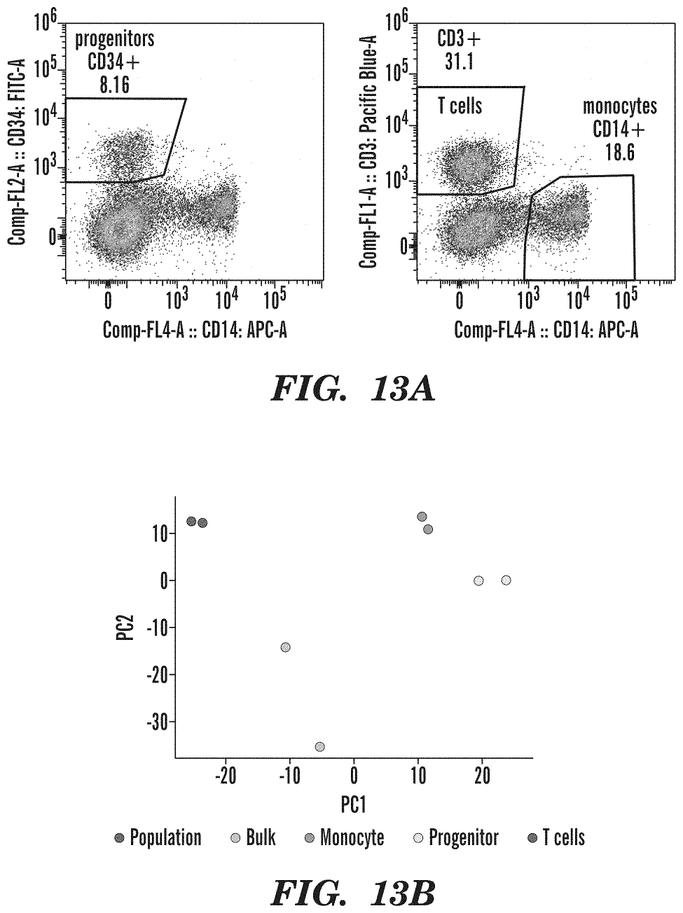

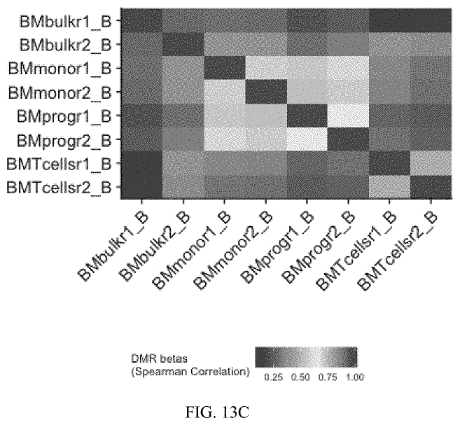

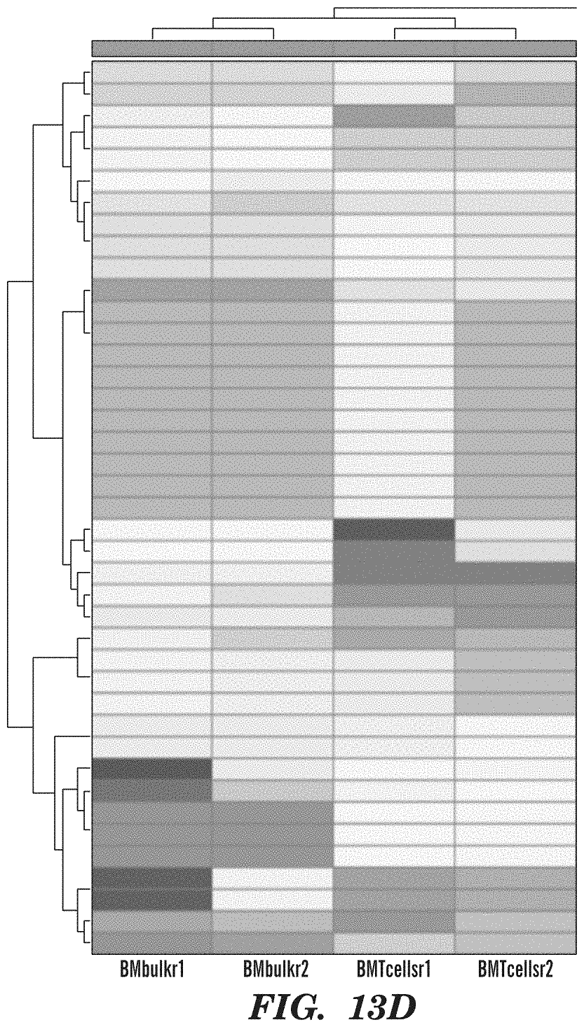

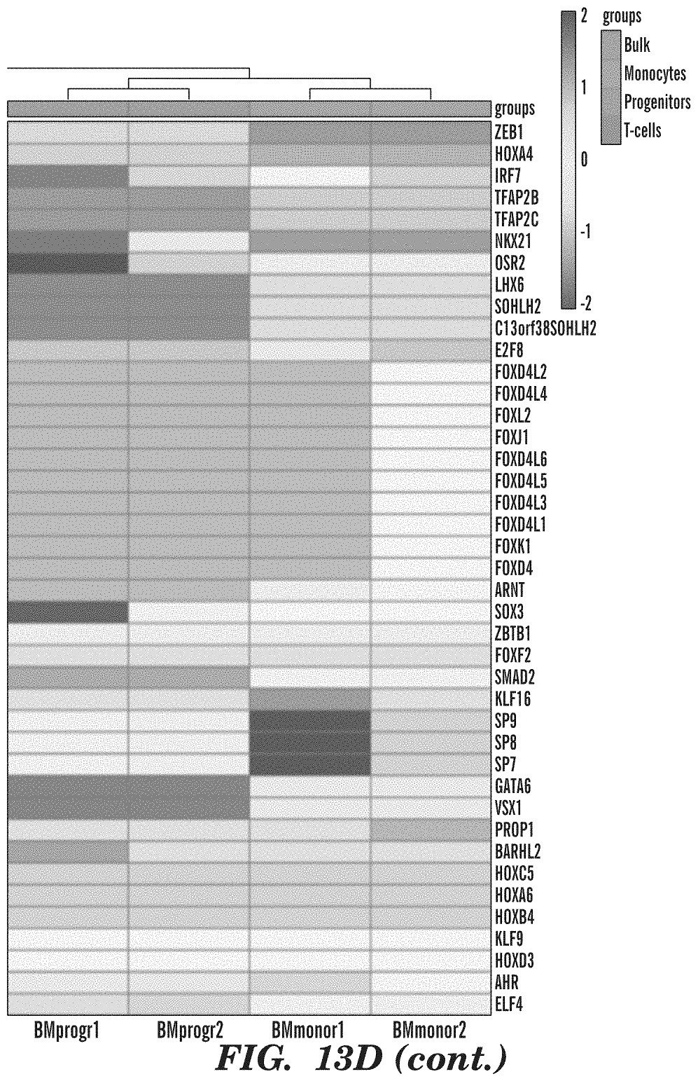

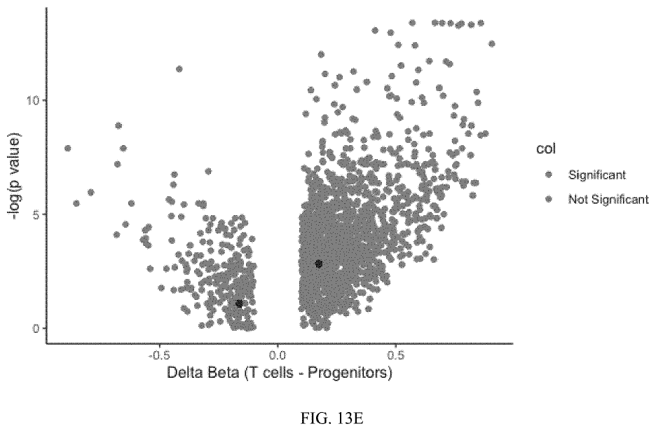

Cells present in a bone marrow sample comprise many distinct cell populations (FIG. 12). T cells, progenitor cells, and monocytes were sorted by gating for CD34, CD14, and CD3 (FIG. 13A). DNA methylation was assessed in the sorted cells, and principal component analysis revealed that cells from each distinct population (i.e., T cells, monocytes, and progenitor cells), as well as the entire population of cells, had different profiles of differentially methylated regions (FIG. 13B). The profiles of cells from the same lineage clustered together, allowing discrimination between cell types based on their methylation profiles (FIG. 13C). Furthermore, the methylation profiles of a subset of transcription factors were distinct for different cell types (and bulk methylation data) (FIG. 13D), which indicates that the present method can distinguish between single cells based on their methylation profile and can be useful in characterizing the regulation of gene expression in cells. For example, the differentially methylated regions (DMRs) of progenitor cells were distinct from those in T cells. The DMRs in T cells were hypermethylated, whereas those of the progenitor cells were not (FIG. 13E). Metilene software was used to call differentially methylated regions between T cells and progenitors from the duplicate methylation profiles.

The results reported herein above were obtained using the following materials and methods.

### Materials and Methods for Examples 1 to 6:

Reagents for the preparation of methylation libraries: MspI; T4 DNA Ligase; 10 mM ATP; 10× NEB Buffer 2.I; 10 mM dNTP mix; 2× KAPA HiFi U+ Master Mix.

### Oligonucleotides

All oligos were ordered from IDT. Custom adapters were designed with a methylated top strand and biotinylated bottom strand. An 8 bp C-depleted barcode precedes the MspI cut site. These paired adaptors are listed in Table 1. A random hexamer oligo is used for the Klenow extension. Oligos for the final library PCR for compatibility with Illumina were generously provided by Peter van Galen.

### Antibodies

Normal human bone marrow cells were stained for cell surface markers for 20 minutes on ice. Antibodies used and concentrations are provided in Table 2.

Flow Sorting

Cell lines (1-5 million) were counterstained with 1:1000 μL of propidium iodide in PBS and sorted in Sony SH800 sorter. Viable single cells were gated and used for sorting into 96 well plates.

TABLE 1
Single Cell Barcodes-Paired Adapters with overhang

| Barcode | Location | Sequence |
|---|---|---|
| A | Top | /5SpC3/GGAGTT/iMe-dC/AGA/iMe-dC/GTGTG/iMe-dC/T/iMe-dC/TT/iMe- |
| | Bottom | CGCTATACCAAGATCGGAAGAGCACACGTCTGAACTCC/3Bio/ |
| B | Top | /5SpC3/GGAGTT/iMe-dC/AGA/iMe-dC/GTGTG/iMe-dC/T/iMe-dC/TT/iMe- |
| | Bottom | CGAACCATCCAGATCGGAAGAGCACACGTCTGAACTCC/3Bio/ |
| C | Top | /5SpC3/GGAGTT/iMe-dC/AGA/iMe-dC/GTGTG/iMe-dC/T/iMe-dC/TT/iMe- |
| | Bottom | CGTAATCTATAGATCGGAAGAGCACACGTCTGAACTCC/3Bio/ |
| D | Top | /5SpC3/GGAGTT/iMe-dC/AGA/iMe-dC/GTGTG/iMe-dC/T/iMe-dC/TT/iMe- |
| | Bottom | CGACCTCTTCAGATCGGAAGAGCACACGTCTGAACTCC/3Bio/ |
| E | Top | /5SpC3/GGAGTT/iMe-dC/AGA/iMe-dC/GTGTG/iMe-dC/T/iMe-dC/TT/iMe- |
| | Bottom | CGCAAATCTCAGATCGGAAGAGCACACGTCTGAACTCC/3Bio/ |
| F | Top | /5SpC3/GGAGTT/iMe-dC/AGA/iMe-dC/GTGTG/iMe-dC/T/iMe-dC/TT/iMe- |
| | Bottom | CGCAACCTCCAGATCGGAAGAGCACACGTCTGAACTCC/3Bio/ |
| G | Top | /5SpC3/GGAGTT/iMe-dC/AGA/iMe-dC/GTGTG/iMe-dC/T/iMe-dC/TT/iMe- |
| | Bottom | CGCCTTTAATAGATCGGAAGAGCACACGTCTGAACTCC/3Bio/ |
| H | Top | /5SpC3/GGAGTT/iMe-dC/AGA/iMe-dC/GTGTG/iMe-dC/T/iMe-dC/TT/iMe- |
| | Bottom | CGATACCAAAAGATCGGAAGAGCACACGTCTGAACTCC/3Bio/ |
| I | Top | /5SpC3/GGAGTT/iMe-dC/AGA/iMe-dC/GTGTG/iMe-dC/T/iMe-dC/TT/iMe- |
| | Bottom | CGAACCTCTTAGATCGGAAGAGCACACGTCTGAACTCC/3Bio/ |
| J | Top | /5SpC3/GGAGTT/iMe-dC/AGA/iMe-dC/GTGTG/iMe-dC/T/iMe-dC/TT/iMe- |
| | Bottom | CGCTATTATAAGATCGGAAGAGCACACGTCTGAACTCC/3Bio/ |
| K | Top | /5SpC3/GGAGTT/iMe-dC/AGA/iMe-dC/GTGTG/iMe-dC/T/iMe-dC/TT/iMe- |
| | Bottom | CGAATACCAAAGATCGGAAGAGCACACGTCTGAACTCC/3Bio/ |
| L | Top | /5SpC3/GGAGTT/iMe-dC/AGA/iMe-dC/GTGTG/iMe-dC/T/iMe-dC/TT/iMe- |
| | Bottom | CGAACACTTTAGATCGGAAGAGCACACGTCTGAACTCC/3Bio/ |
| M | Top | /5SpC3/GGAGTT/iMe-dC/AGA/iMe-dC/GTGTG/iMe-dC/T/iMe-dC/TT/iMe- |
| | Bottom | CGACACACTCAGATCGGAAGAGCACACGTCTGAACTCC/3Bio/ |
| N | Top | /5SpC3/GGAGTT/iMe-dC/AGA/iMe-dC/GTGTG/iMe-dC/T/iMe-dC/TT/iMe- |
| | Bottom | CGCTTAACCAAGATCGGAAGAGCACACGTCTGAACTCC/3Bio/ |
| O | Top | /5SpC3/GGAGTT/iMe-dC/AGA/iMe-dC/GTGTG/iMe-dC/T/iMe-dC/TT/iMe- |
| | Bottom | CGTAATCCTTAGATCGGAAGAGCACACGTCTGAACTCC/3Bio/ |
| P | Top | /5SpC3/GGAGTT/iMe-dC/AGA/iMe-dC/GTGTG/iMe-dC/T/iMe-dC/TT/iMe- |
| | Bottom | CGTACTAAACAGATCGGAAGAGCACACGTCTGAACTCC/3Bio/ |
| Q | Top | /5SpC3/GGAGTT/iMe-dC/AGA/iMe-dC/GTGTG/iMe-dC/T/iMe-dC/TT/iMe- |
| | Bottom | CGATCATTTAAGATCGGAAGAGCACACGTCTGAACTCC/3Bio/ |
| R | Top | /5SpC3/GGAGTT/iMe-dC/AGA/iMe-dC/GTGTG/iMe-dC/T/iMe-dC/TT/iMe- |
| | Bottom | CGCCTATCTAAGATCGGAAGAGCACACGTCTGAACTCC/3Bio/ |
| S | Top | /5SpC3/GGAGTT/iMe-dC/AGA/iMe-dC/GTGTG/iMe-dC/T/iMe-dC/TT/iMe- |
| | Bottom | CGAACCCATCAGATCGGAAGAGCACACGTCTGAACTCC/3Bio/ |
| T | Top | /5SpC3/GGAGTT/iMe-dC/AGA/iMe-dC/GTGTG/iMe-dC/T/iMe-dC/TT/iMe- |
| | Bottom | CGACTCTACCAGATCGGAAGAGCACACGTCTGAACTCC/3Bio/ |
| U | Top | /5SpC3/GGAGTT/iMe-dC/AGA/iMe-dC/GTGTG/iMe-dC/T/iMe-dC/TT/iMe- |
| | Bottom | CGTATTACATAGATCGGAAGAGCACACGTCTGAACTCC/3Bio/ |
| V | Top | /5SpC3/GGAGTT/iMe-dC/AGA/iMe-dC/GTGTG/iMe-dC/T/iMe-dC/TT/iMe- |
| | Bottom | CGACTACACCAGATCGGAAGAGCACACGTCTGAACTCC/3Bio/ |
| W | Top | /5SpC3/GGAGTT/iMe-dC/AGA/iMe-dC/GTGTG/iMe-dC/T/iMe-dC/TT/iMe- |
| | Bottom | CGATCTTTATAGATCGGAAGAGCACACGTCTGAACTCC/3Bio/ |
| X | Top | /5SpC3/GGAGTT/iMe-dC/AGA/iMe-dC/GTGTG/iMe-dC/T/iMe-dC/TT/iMe- |
| | Bottom | CGATATCCCAAGATCGGAAGAGCACACGTCTGAACTCC/3Bio/ |

“5SpC3” denotes 5′ C3 spacer.

TABLE 2
Antibodies

| Antibody | Company | Number | Dilution |
|---|---|---|---|
| CD34-FITC | BD Biosciences | 348053 | 1:100 |
| CD3-Pacific Blue | BD Biosciences | 558124 | 1:100 |
| CD14-APC | “BD” | P0756L | 1:100 |

Normal human bone marrow was collected and stored in liquid nitrogen. Frozen bone marrow was thawed in 37° C. water bath and transferred to a 15-mL conical. 9 mL of thawing medium (RPMI 50% FBS, P/S) was added slowly over 3-5 minutes while carefully agitating tube. The resuspended cells were spun at 300 Relative Centrifugal Force (rcf) for 5 minutes and then resuspended in 1 mL of RPMI(+10% FBS, P/S). Viable cells were counted and cells were aliquoted.

Unstained and PI only controls were generated with 50-100K cells. Antibody controls were created with 100 μL of beads and 1 μL of each of the 3 antibodies used in the experiment. The remaining cells were spun and resuspended at 1-5 million cells/mL in PBS+2% FBS. All 3 antibodies were added at concentrations listed in table 1.3 and incubated on ice for 20 minutes. Then the cells were spun down and washed in 4 mL of PBS+2% FBS and finally resuspended in PBS and 2% FBS with PI (1:2000) at 1-5 million cells per mL and sorted on a Sony SH800 Sorter. The antibody-bead controls were used for manual compensation.

The goal in lysing the cells was to isolate all DNA and remove any representation bias towards open chromatin. Starting the protocol with nuclei or with cells was considered. Isolating nuclei first would make the method easy to apply to frozen tissues as well as paraffin blocks of tissue. Thus, the Zymo Nuclei Isolation Kit was used to recover nuclei and sort nuclei into wells. The wells contained 3 μL of proK, 10% SDS, and 100 mM NaCl.

The advantage of an input of cells is the ability to sort according to cell surface markers. The effectiveness of cellular lysis using Tris to generate an ATAC library was tested. Cells lysed using that described in the LIANTI method, they showed no enrichment for open chromatin typical of ATAC libraries

### Example 7

### A Targeted DNA Methylation Profiling Method Compatible with Low Input Samples

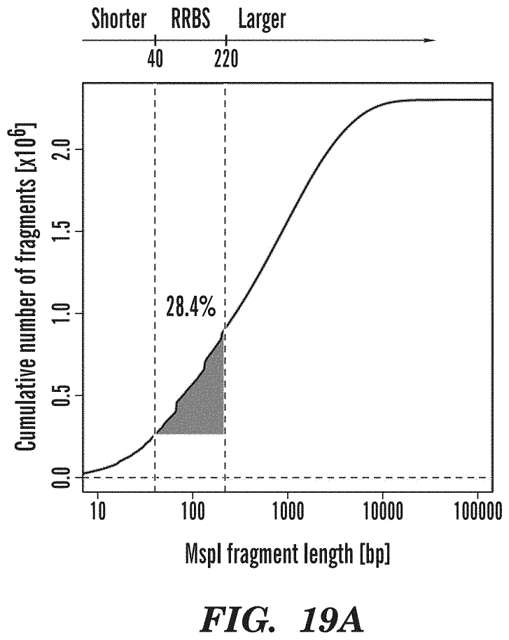

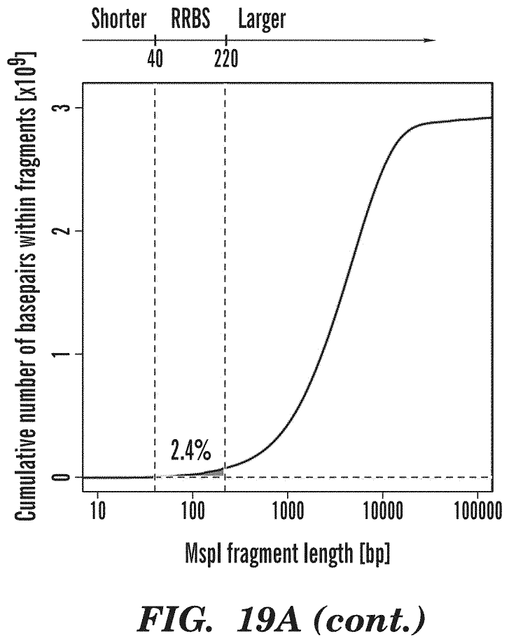

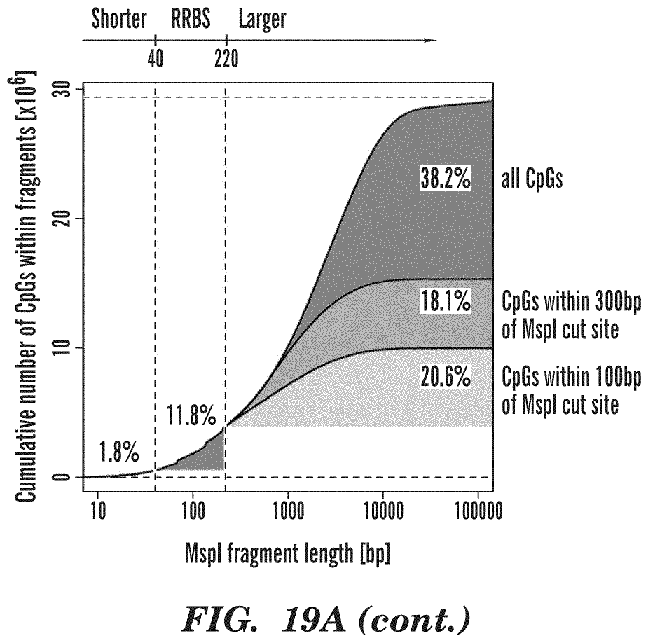

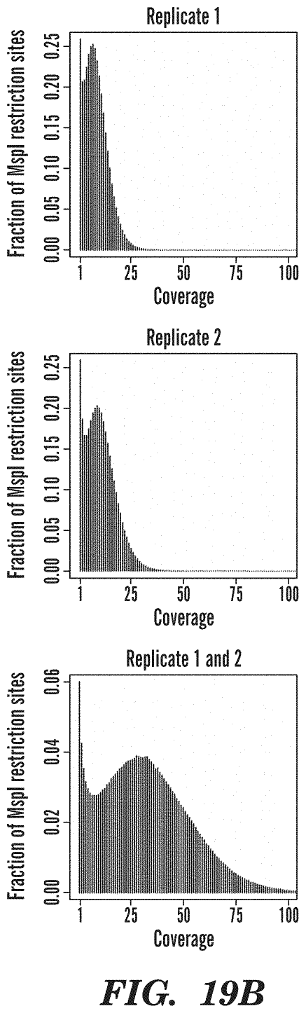

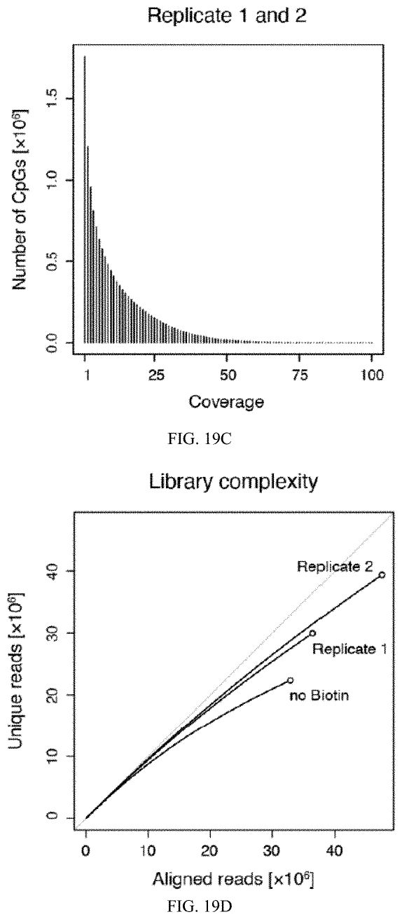

A DNA methylation assay was designed with expanded coverage. The method included promoters, enhancers, and other regulatory elements that allow multiplexing and analysis of low-input samples. The method enriched CpG-containing regions for efficiency, but was not limited to CpG island promoters. Reduced representation bisulfite sequencing (RRBS) enriched CpG dense loci by purifying short genomic fragments after restriction by the methylation-insensitive enzyme MspI (cuts CCGG). Thus, reduced representation bisulfite sequencing (RRBS) captured fragments flanked by two proximate MspI sites (40-220 bases) on both ends. It was tested whether a procedure that also captured sequences flanked by a single MspI site could expand functional element coverage. In-silico analysis indicated that such a method can capture significantly more CpG dinucleotides: considering all CpGs within 300 bases of an MspI site, the method can theoretically cover 14.8 million CpGs or 50.5% of all CpGs in the human genome, compared to 11.8% of CpGs covered by reduced representation bisulfite sequencing (RRBS) (FIG. 19A).

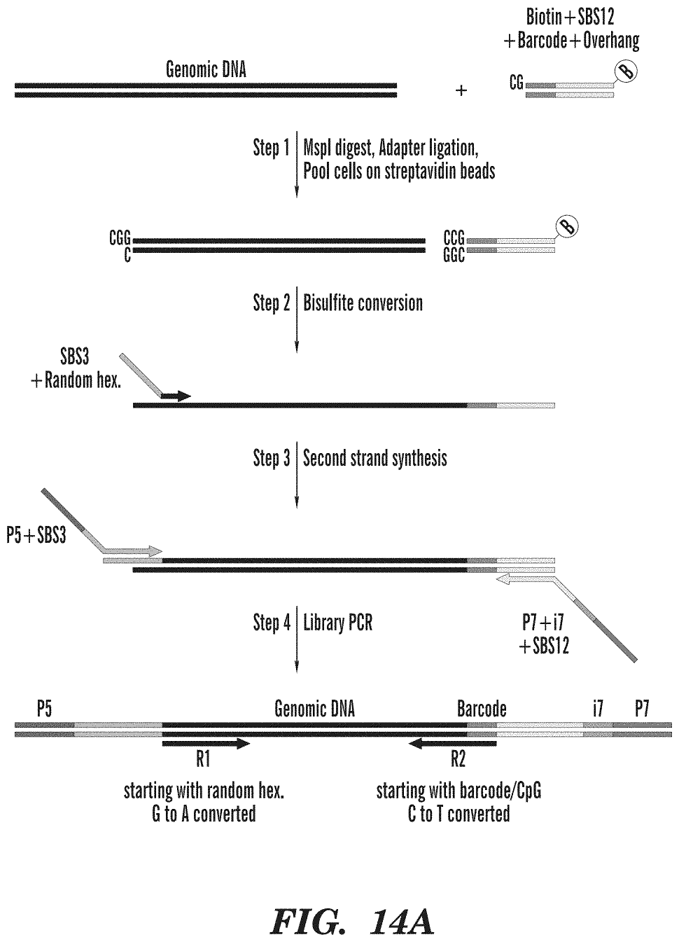

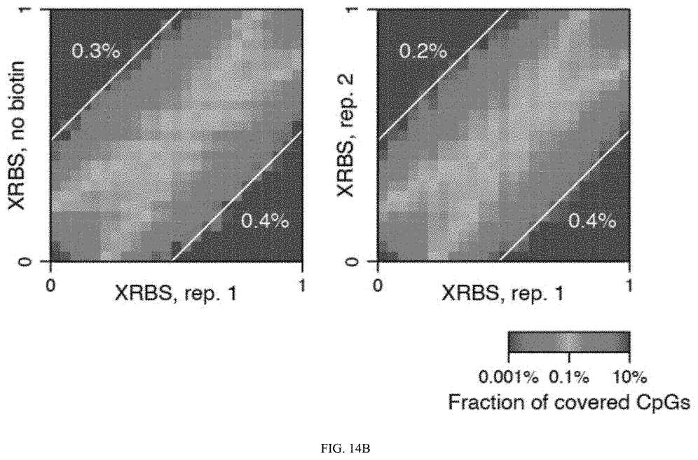

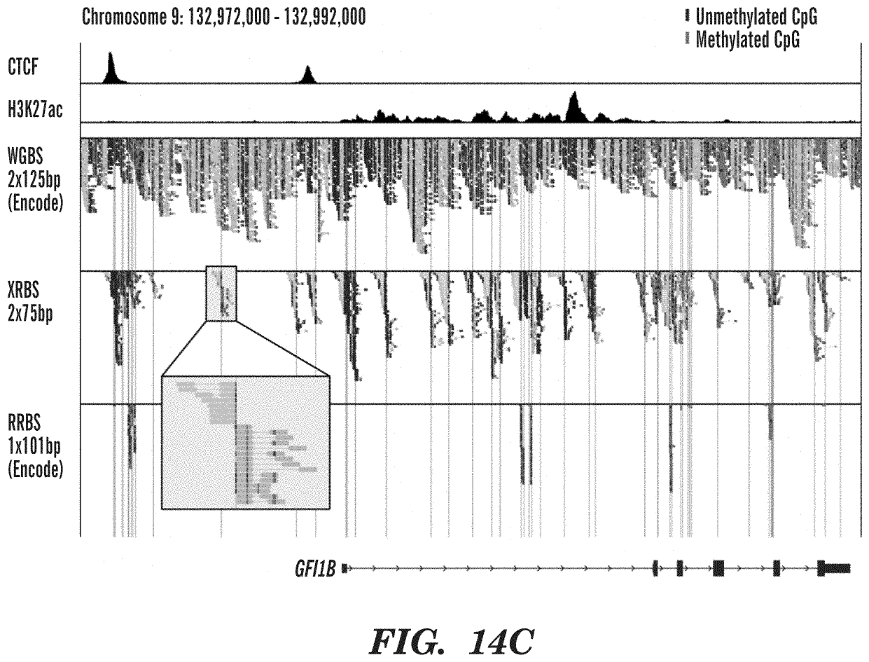

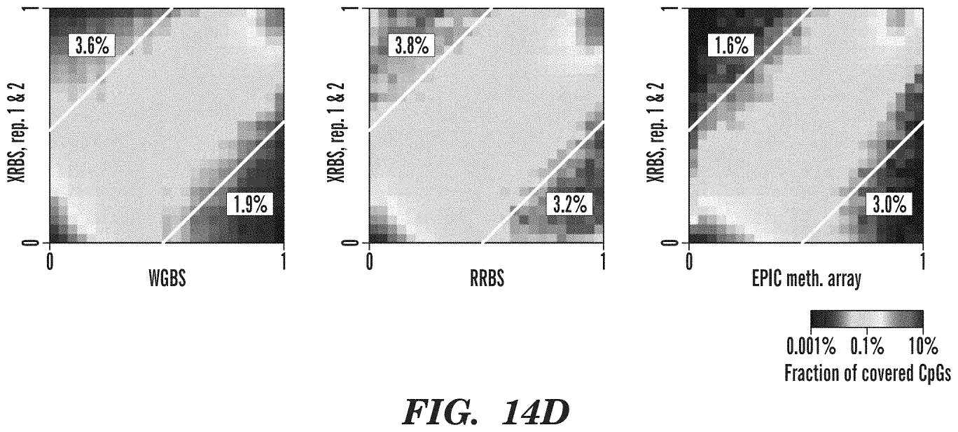

This strategy was implemented in the following experimental design. First, a one-step incubation was optimized that combined MspI restriction and ligation of the restricted genomic fragments to adapters containing sample-identifying barcodes (FIG. 14A). Second, samples were pooled, adapted fragments were captured, and excess volume was removed in a biotin-enrichment step prior to bisulfite conversion. The barcoded adapters were designed with a methylated top strand (bisulfite-protected) and a bottom strand that was unphosphorylated (preventing the formation of a covalent bond) and biotinylated. This enabled efficient release of fragments during a combined bisulfite conversion reaction. Third, a random hexamer extension step was used to incorporate a second adapter sequence (FIG. 14A). This approach expanded coverage to genomic sequences with isolated MspI sites, and recovered degraded fragments generated during bisulfite conversion and prevalent in low-quality DNA samples, for example from formalin-fixed paraffin-embedded (FFPE) tissues. In addition, the hexamer sequences often primed imperfect matches and could thus be used as a unique molecular identifier (UMI) to identify duplicate reads originating from library polymerase chain reaction (PCR) (see Materials and Methods for Examples 7 to 11 below).

Libraries were generated from 10 ng of purified genomic DNA from K562 cells and technical replicate libraries were sequenced to ˜40 million paired-end reads (FIGS. 19B to 19C). In addition, another replicate was generated without the biotin enrichment step. High concordance was confirmed between all three replicates (r=0.96-0.97; FIG. 14B) and comparable library complexity was confirmed in libraries with and without biotin (FIG. 19D), ruling out negative effects of biotin on adapter ligation efficiency.

Next, the extended representation bisulfite-sequencing method (expanded representation bisulfite sequencing (XRBS)) was compared to whole genome bisulfite sequencing (WGBS) and reduced representation bisulfite sequencing (RRBS). FIG. 14C illustrates a representative region at the GFI1B gene locus. While a high coverage whole genome bisulfite sequencing (WGBS) profile showed uniform coverage across the entire 20 kb region, read coverage from expanded representation bisulfite sequencing (XRBS) was enriched around individual MspI sites. Expanded representation bisulfite sequencing (XRBS) achieved much higher coverage of the region than reduced representation bisulfite sequencing (RRBS), including a 10 kb intronic enhancer and multiple CTCF-binding sites. Expanded representation bisulfite sequencing (XRBS) reads mapped to various positions up and downstream of isolated MspI restriction sites, explaining the expanded coverage (FIG. 14C). DNA Methylation values of individual CpGs correlate well between expanded representation bisulfite sequencing (XRBS), whole genome bisulfite sequencing (WGBS), reduced representation bisulfite sequencing (RRBS), and EPIC methylation array datasets (r=0.90-0.91; FIG. 14D). Taken together, expanded representation bisulfite sequencing (XRBS) generates accurate DNA methylation profiles, expands coverage of functional elements flanked by a single MspI site and is compatible with pre-bisulfite sample multiplexing.

### Example 8

### Expanded Representation Bisulfite Sequencing (XRBS) Expands Coverage and Enriches Over Enhancers and CTCF Binding Regions

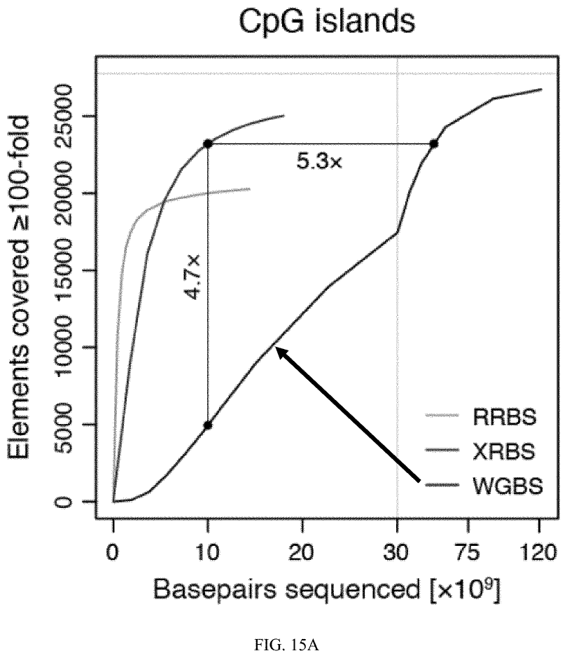

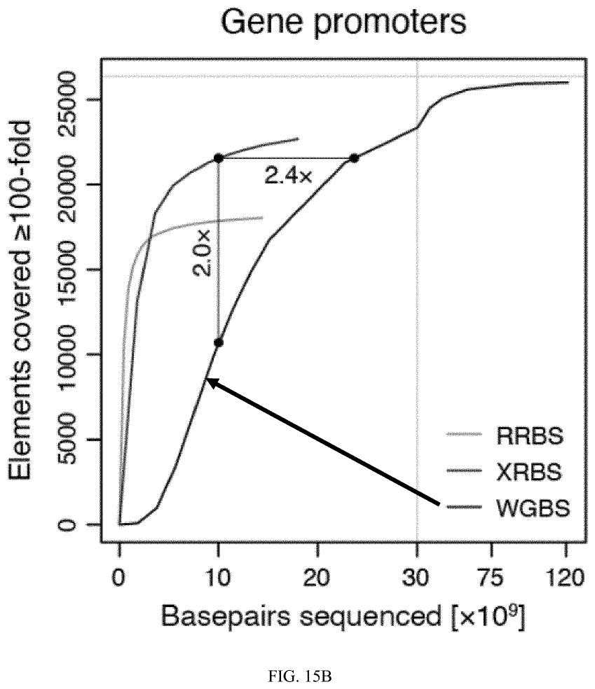

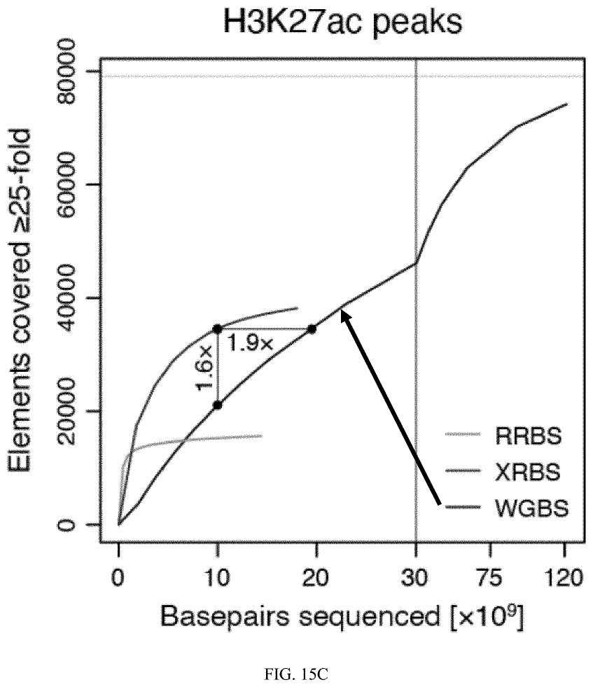

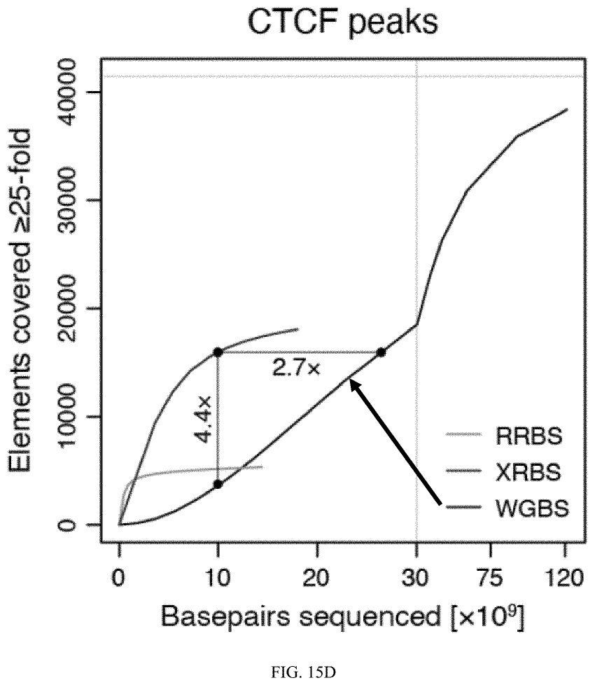

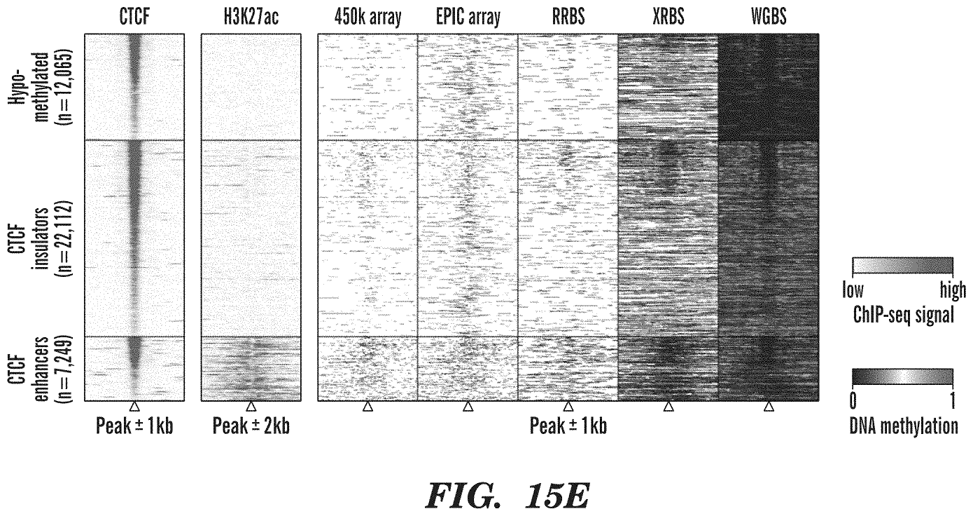

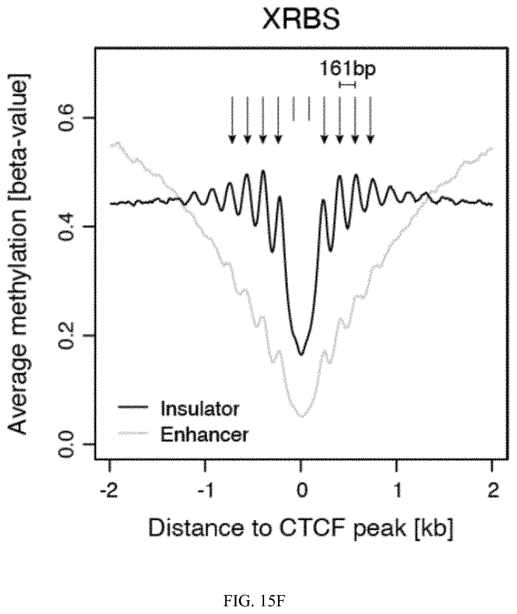

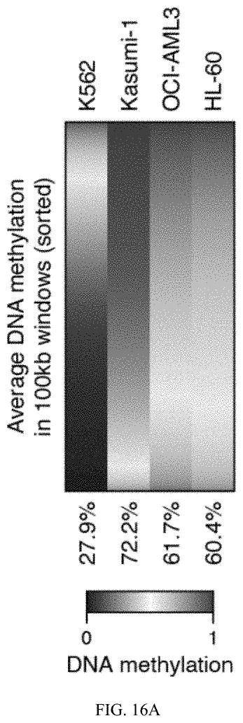

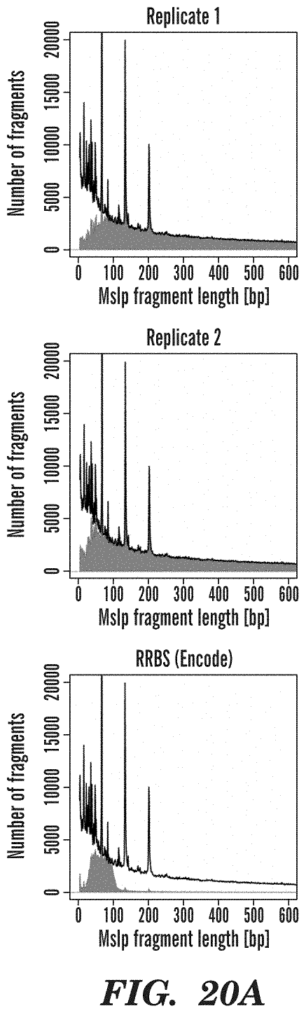

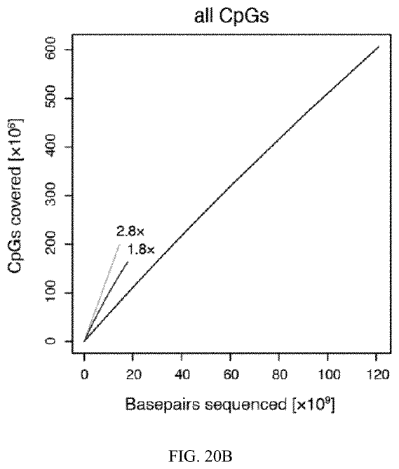

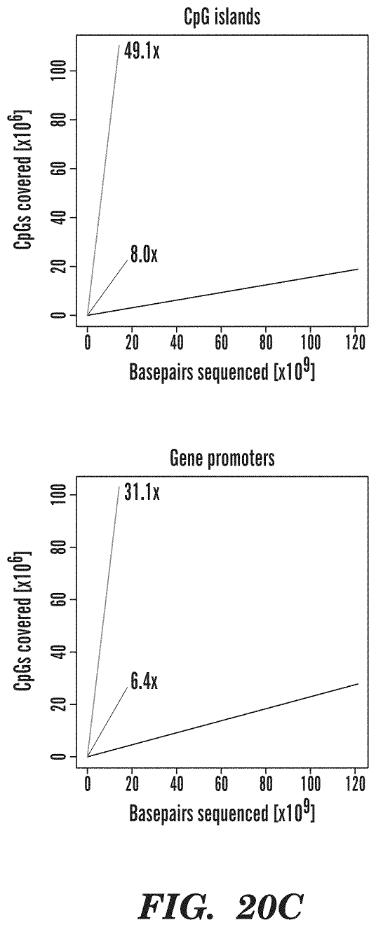

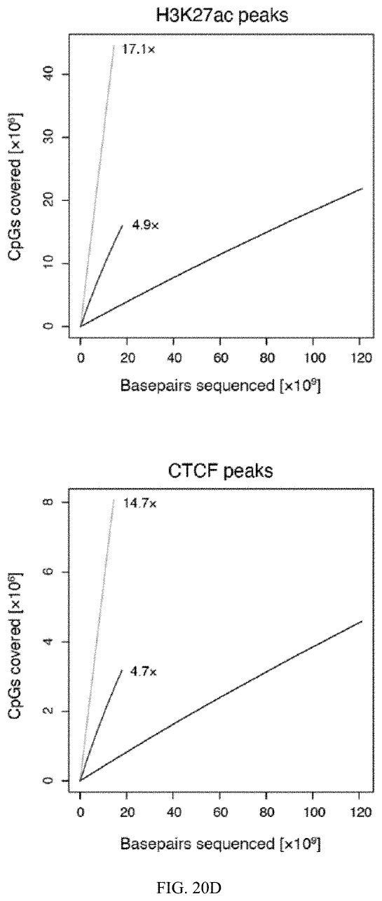

To evaluate coverage of functionally-relevant genomic regions, coverage and enrichment over elements such as CpG islands, gene promoters, enhancers, and CTCF binding sites were systematically compared. DNA methylation changes in each of these regions has been linked to transcriptional regulation. Genomic coverage of these elements was compared between expanded representation bisulfite sequencing (XRBS), whole genome bisulfite sequencing (WGBS) and reduced representation bisulfite sequencing (RRBS). For purposes of comparison, each dataset was downsampled to 10 billion base pairs of sequencing data (corresponding to 66.7 million 75 bp paired-end reads), and considered an element to be covered if all contained CpGs accumulated to ≥100-fold coverage. With these criteria, expanded representation bisulfite sequencing (XRBS) and reduced representation bisulfite sequencing (RRBS) respectively captured 83.5% and 72.0% of CpG islands (FIGS. 15A, and 20A to 20C). Whole genome bisulfite sequencing (WGBS) captured a lower fraction (17.8%) at this sequencing coverage since it does not enrich for CpG-rich regions. Achieving similar coverage of CpG islands by whole genome bisulfite sequencing (WGBS) would require ˜5.3-fold deeper sequencing. Of note, the sample input amount required for whole genome bisulfite sequencing (WGBS) profiling is orders of magnitude higher than that for expanded representation bisulfite sequencing (XRBS) (10 ng). Similarly, at a sequencing depth of 10 billion base pairs, expanded representation bisulfite sequencing (XRBS) reduced representation bisulfite sequencing (RRBS) and whole genome bisulfite sequencing (WGBS) captured 81.7%, 67.7%, and 40.3% of gene promoters, respectively (FIG. 15B). Achieving a similar coverage by whole genome bisulfite sequencing (WGBS) would require ˜2.4-fold deeper sequencing than with expanded representation bisulfite sequencing (XRBS). When sequenced to saturation (˜18 billion base pairs, corresponding to 120 million 75 bp paired-end reads), expanded representation bisulfite sequencing (XRBS) captured 25,025 CpG islands (90.2%) and 22,684 gene promoters (86.1%).

Coverage of enhancers and CTCF binding sites was next considered. Not wishing to be bound by theory, DNA methylation over enhancers has been shown to negatively correlate with target gene expression. CTCF is a DNA-binding protein with roles in nuclear architecture. DNA methylation antagonizes CTCF binding at CTCF motifs, which are typically found in regions of moderate to low CpG density. When sequenced to saturation, expanded representation bisulfite sequencing (XRBS) provided at least 25-fold coverage for 38,211 enhancers and 18,059 CTCF sites, compared to 15,239 enhancers and 5,170 CTCF sites with reduced representation bisulfite sequencing (RRBS) (FIGS. 15C and 20D). At a sequencing depth of 10 billion base pairs, expanded representation bisulfite sequencing (XRBS) captured 1.6-fold more enhancers and 4.4-fold more CTCF sites than whole genome bisulfite sequencing (WGBS). Achieving a similar coverage using whole genome bisulfite sequencing (WGBS) would require ˜1.6-fold and ˜2.7-fold deeper sequencing than with expanded representation bisulfite sequencing (XRBS) for enhancers and CTCF peaks, respectively (FIGS. 15C and 15D).

To analyze how expanded representation bisulfite sequencing (XRBS) performs relative to other DNA methylation profiling technologies in more detail, DNA methylation was visualized around CTCF binding sites (FIG. 15E). It was found that the 10 ng expanded representation bisulfite sequencing (XRBS) profile provided noticeably higher coverage compared to reduced representation bisulfite sequencing (RRBS), Illumina 450 k, and EPIC methylation arrays. This enabled observations of nucleosome occupancy-associated periodicity of DNA methylation relative to CTCF binding sites, as reported previously (FIG. 15F). Notably, this patterning was more pronounced at CTCF binding sites classified as insulators, compared to CTCF binding sites overlapping H3K27ac-positive enhancers. This analysis indicated that expanded representation bisulfite sequencing (XRBS) captures diverse types of regulatory elements with higher efficiency and lower sequencing requirements.

### Example 9

### Expanded Representation Bisulfite Sequencing (XRBS) Detects Differential DNA Methylation Across Cell Types and Treatments

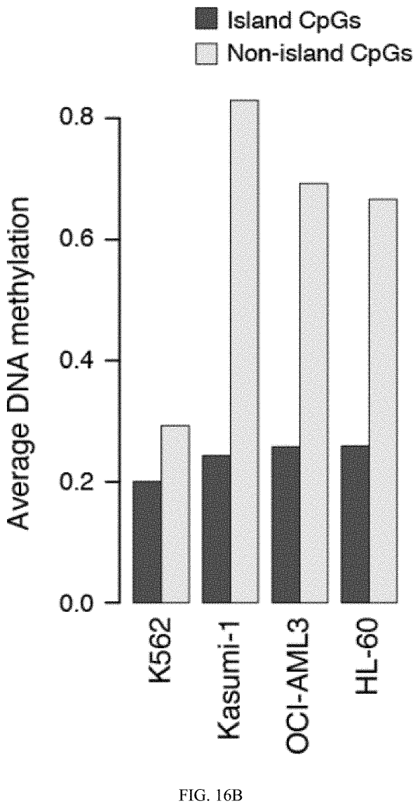

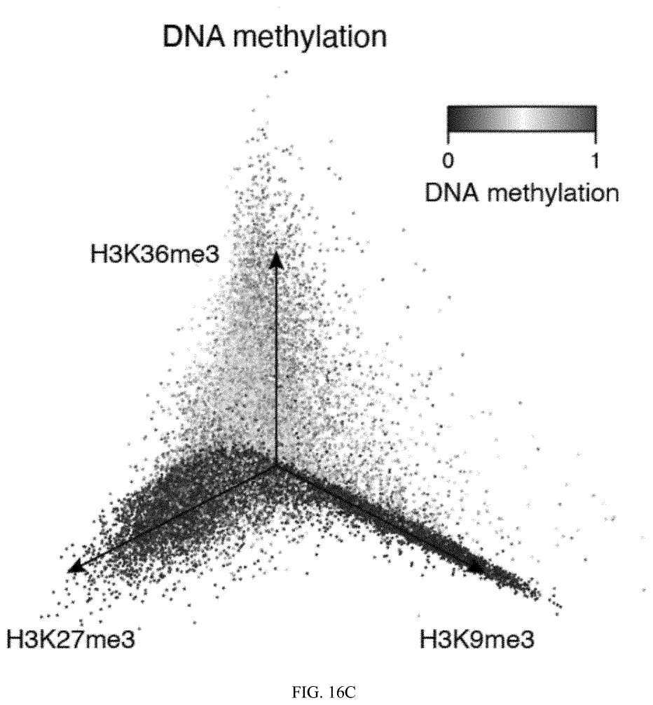

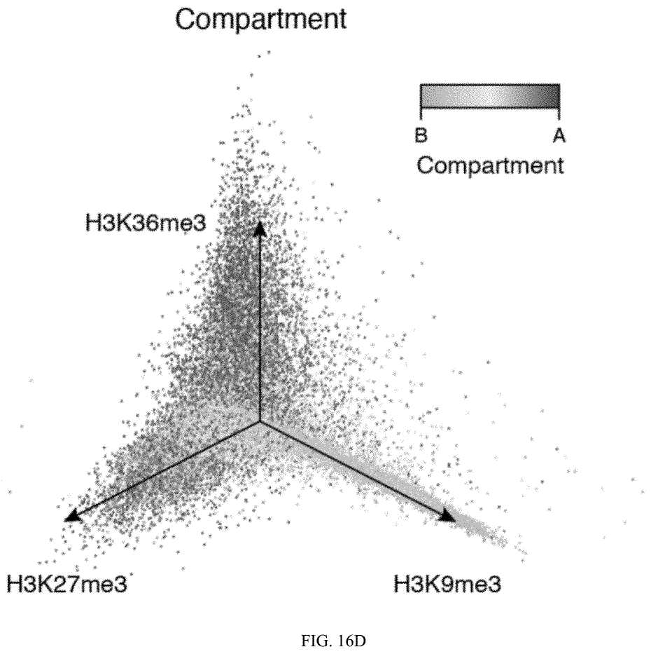

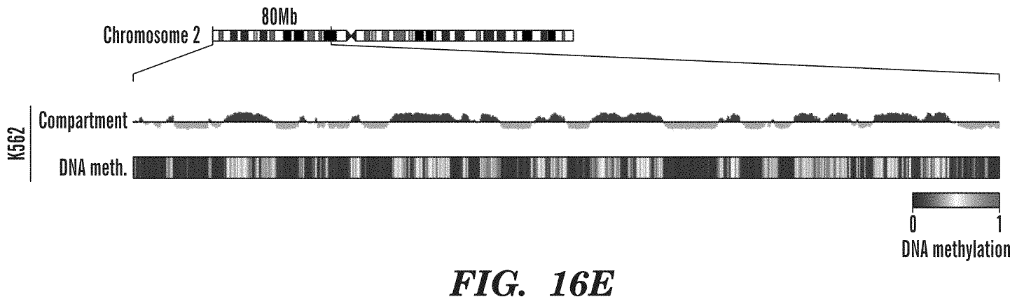

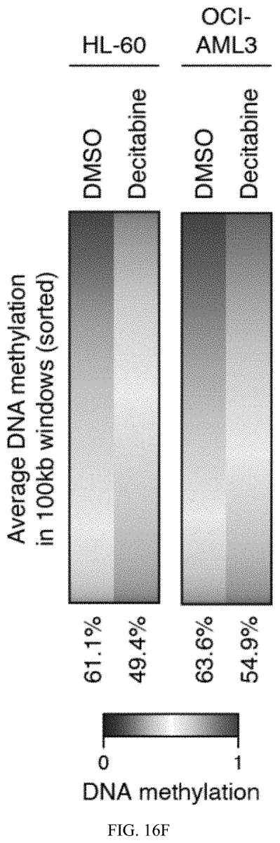

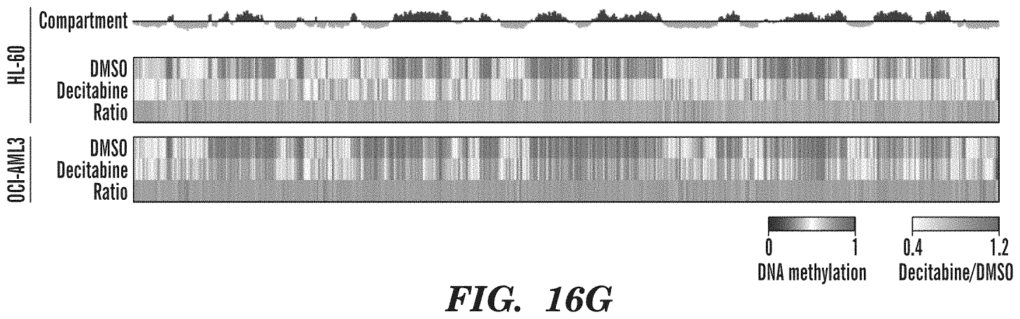

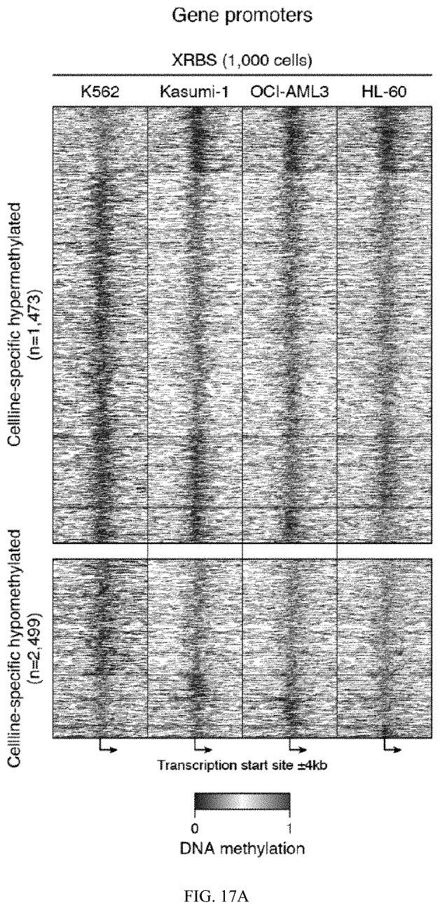

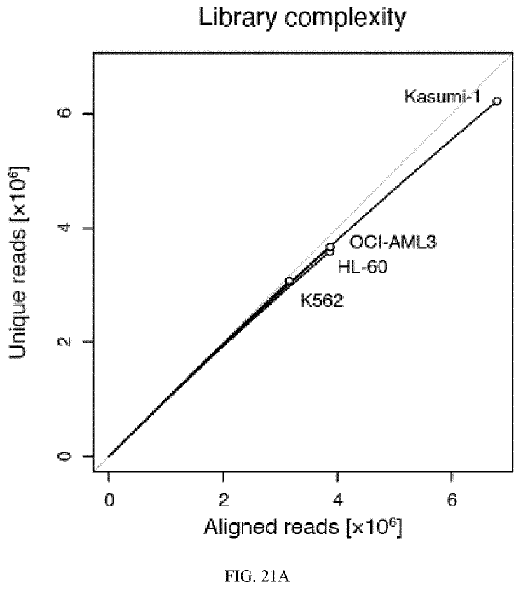

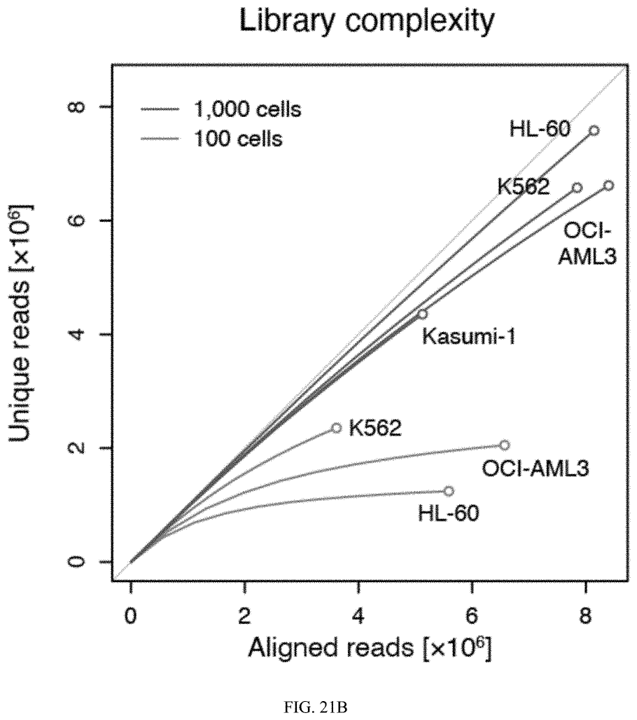

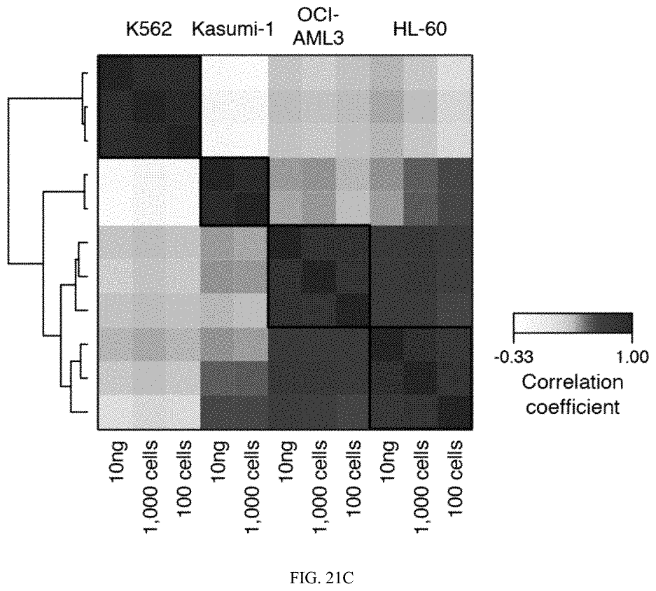

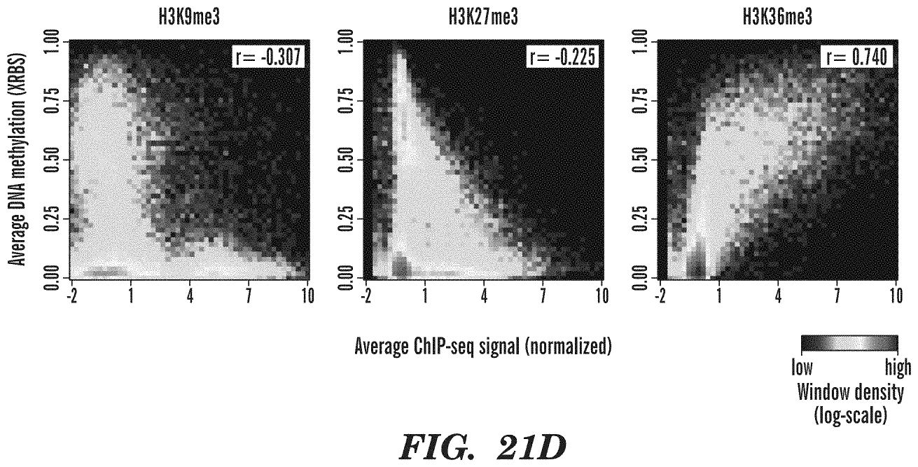

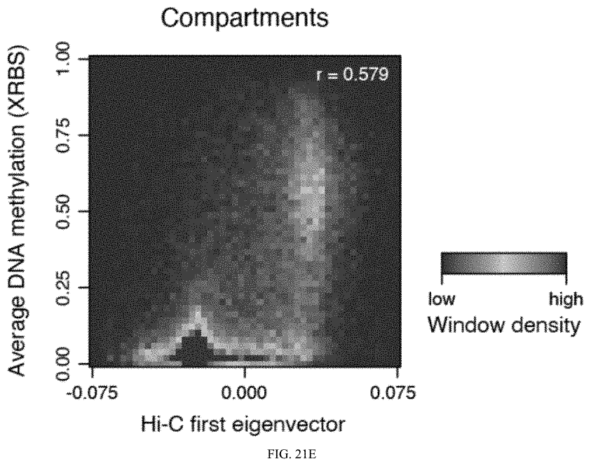

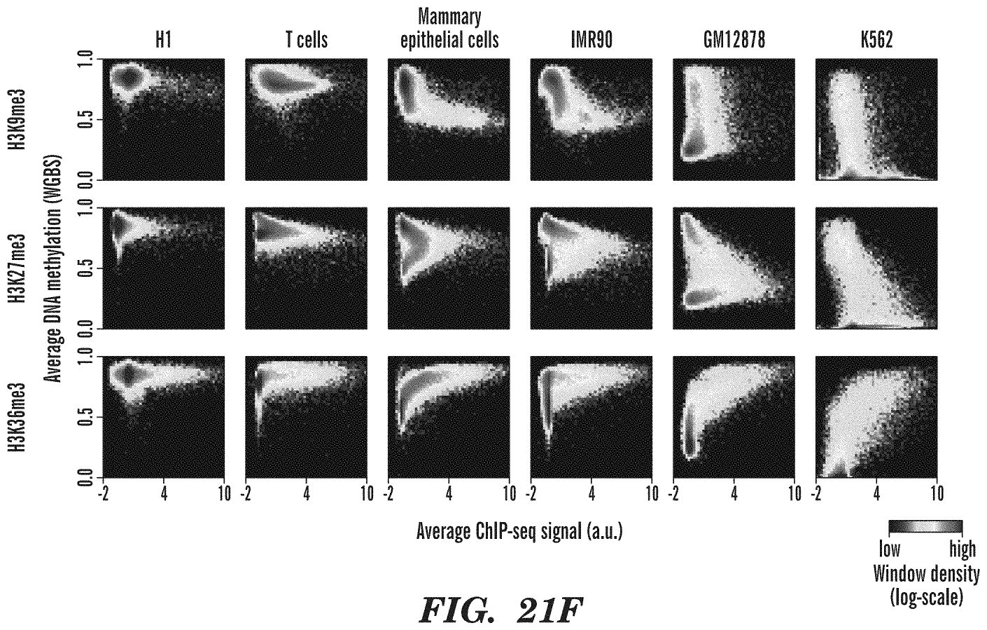

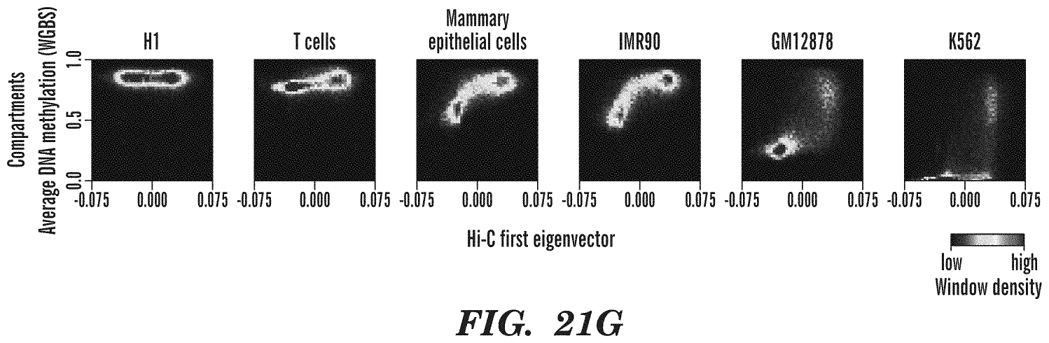

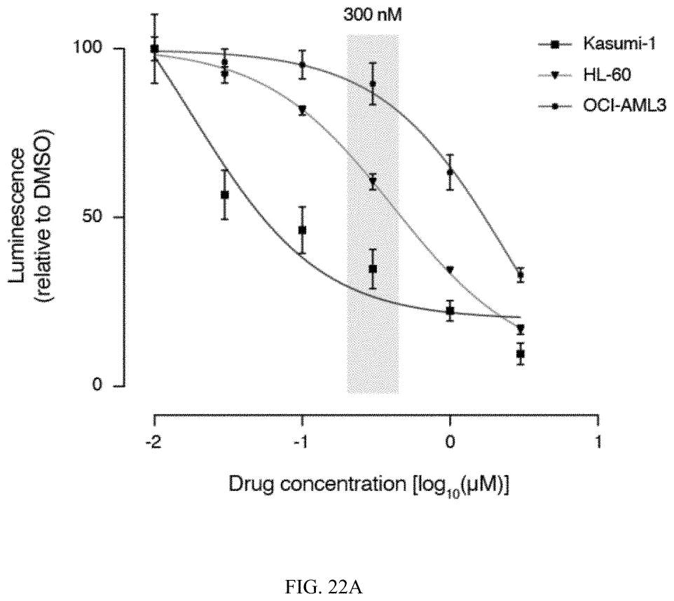

Expanded representation bisulfite sequencing (XRBS) was used to compare methylation patterns across biological samples. In addition to K562 cells, expanded representation bisulfite sequencing (XRBS) libraries were generated for three additional leukemia cell lines (Kasumi-1, OCI-AML3, and HL-60) from 10 ng of purified DNA, as well as 1,000 and 100 cells sorted directly into lysis buffer. Using low-coverage sequencing of ˜5 million paired-end reads per library (FIG. 21A to 21C), it was found that K562 cells were globally hypomethylated (average beta-value=0.28), Kasumi-1 cells were globally hypermethylated (average beta-value=0.72) and OCI-AML3 and HL-60 showed intermediate methylation levels (average beta-value=0.62 and 0.60, respectively; FIG. 16A). These differences in average global DNA methylation were primarily due to non-CpG island CpGs, whereas most CpG islands remained relatively hypomethylated across the cell lines (FIG. 16B).

To contextualize the extensive hypomethylation observed in K562 cells, public data for histone modifications (ChIP-seq) and chromosome topology (Hi-C) was overlaid. Three modifications were considered that mark active transcripts (H3K36me3), facultative heterochromatin (H3K27me3) or constitutive heterochromatin (H3K9me3). Examination of 100 kb-windows revealed that DNA methylation strongly correlated with the active H3K36me3 mark (r=0.74), but negatively correlated with the inactive H3K27me3 (r=−0.31) and H3K9me3 marks (r=−0.23; FIGS. 16C and 21D). Next, genomic compartments called from public Hi-C data were considered. Compartment A is generally associated with active euchromatin, while compartment B represents transcriptionally silent heterochromatin. It was found that compartment B was associated with regions that are H3K9me3-positive and hypomethylated (FIGS. 16D to 16E, and 21E). Hypomethylated regions in K562 were also detected in compartment A, and tended to associate with H3K27me3. In contrast, H3K36me3-positive regions were relatively hypermethylated, consistent with prior studies suggesting that H3K36me3 may protect regions from DNA methylation loss. These results in K562 cells were compared to a broader range of cell types for which public DNA methylation, ChIP-seq and compartment data was available. Limited hypomethylation was observed in H1 embryonic stem cells and primary T-cells (FIGS. 21F and 21G). Primary mammary epithelial cells and cultured IMR90 fibroblasts showed intermediate hypomethylation, with H3K27me3-positive regions being less methylated than H3K36me3-positive regions, but more methylated than H3K9me3-positive regions. Cultured GM12878 cells more closely resembled the extensive hypomethylation of both H3K27me3- and H3K9me3-positive regions observed in K562 cells, indicating that K562 cells exhibit relatively severe hypomethylation even among cultured human cell lines.

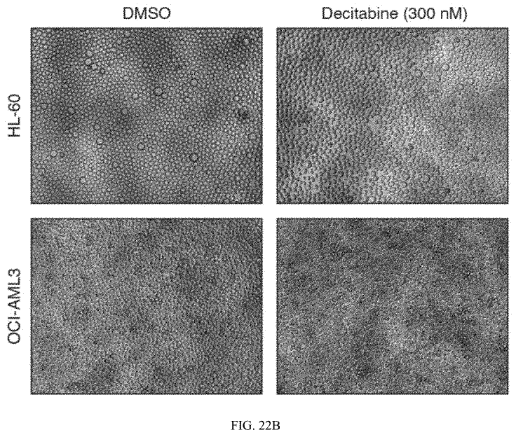

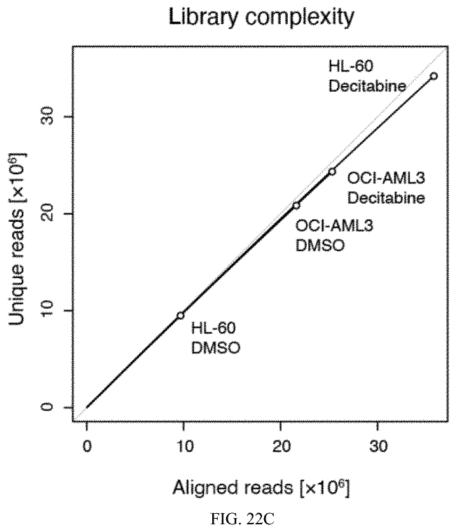

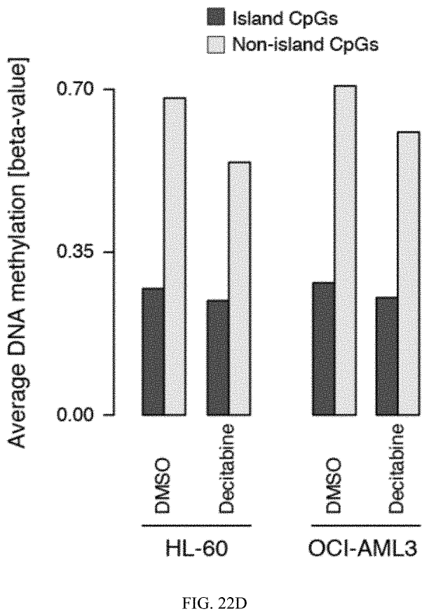

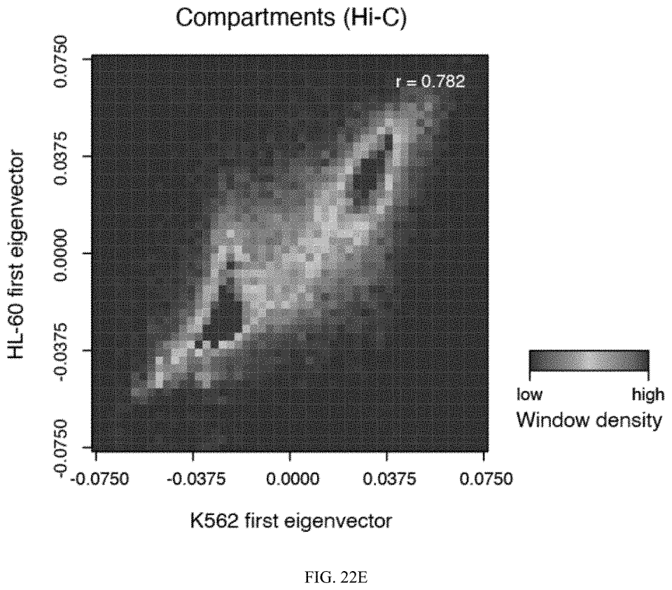

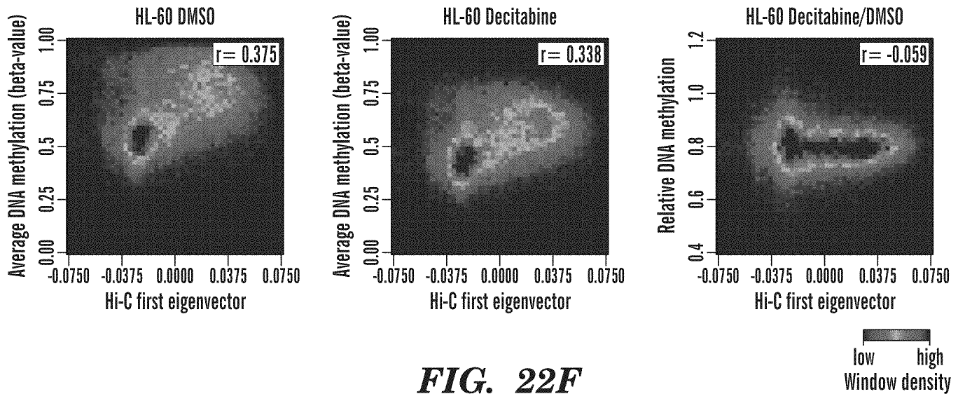

In order to detect differential methylation across an isogenic system, cell lines were treated with 300 nM decitabine, which inhibits DNA methyltransferase enzymes through covalent entrapment on DNA (FIGS. 22A and 22B). HL-60 and OCI-AML3 were treated, as these had intermediate global methylation levels among the four cell lines examined, and profiled DNA methylation using expanded representation bisulfite sequencing (XRBS) (FIG. 22C). Five days of decitabine treatment reduced average global methylation levels by 19.1% in HL-60 and 13.7% in OCI-AML3 (FIG. 16F). DNA methylation levels decreased more dramatically in non-island CpGs (20.2% and 14.0%), while CpG islands were less affected (9.5% and 11.2%; FIG. 22D). Decitabine reduced methylation to a similar extent in both compartment A and B, irrespective of differences in original methylation levels (FIGS. 16G, 22E, and 22F). Low-coverage expanded representation bisulfite sequencing (XRBS) revealed global differences in methylation levels over functional elements between different cell types, and reveals widespread reduction in DNA methylation levels across these elements in response to decitabine treatment.

### Example 10

### Expanded Representation Bisulfite Sequencing (XRBS) Profiles Inform Enhancer States and CTCF Binding

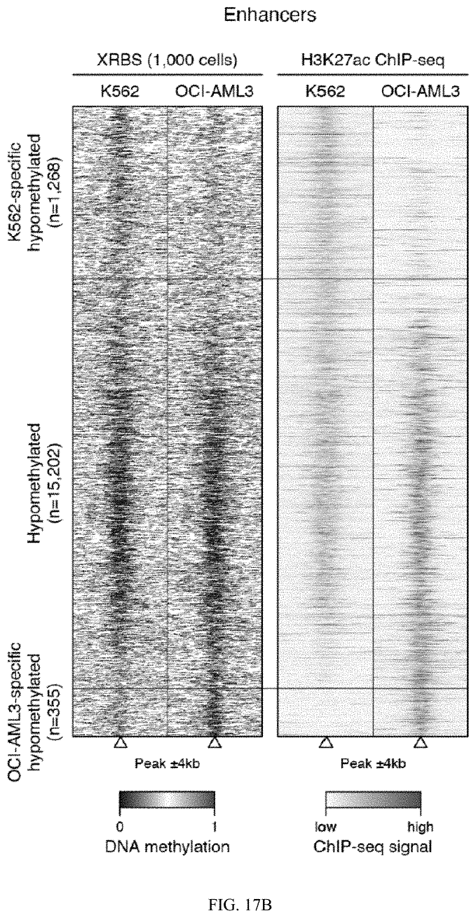

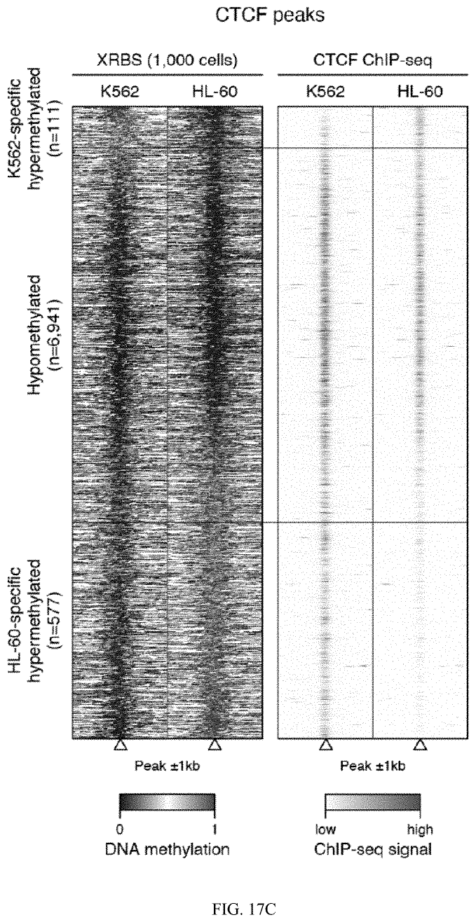

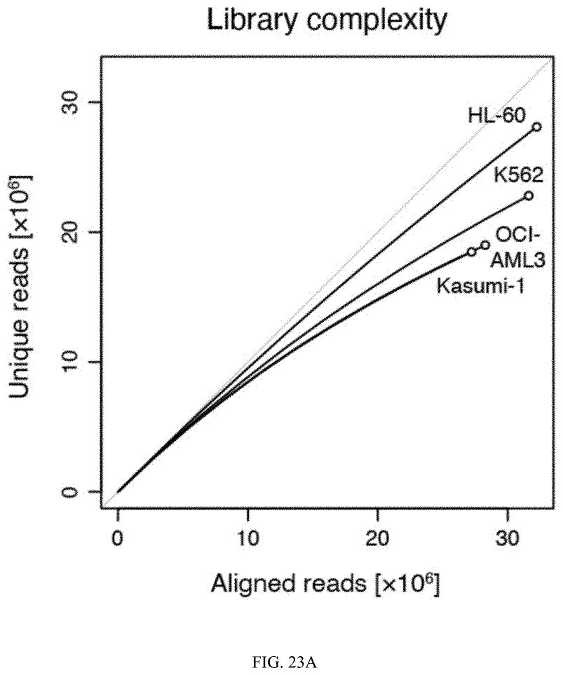

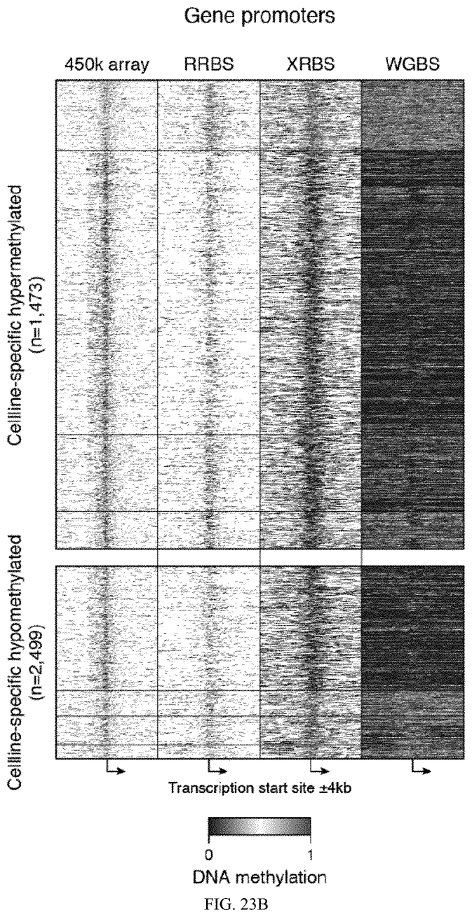

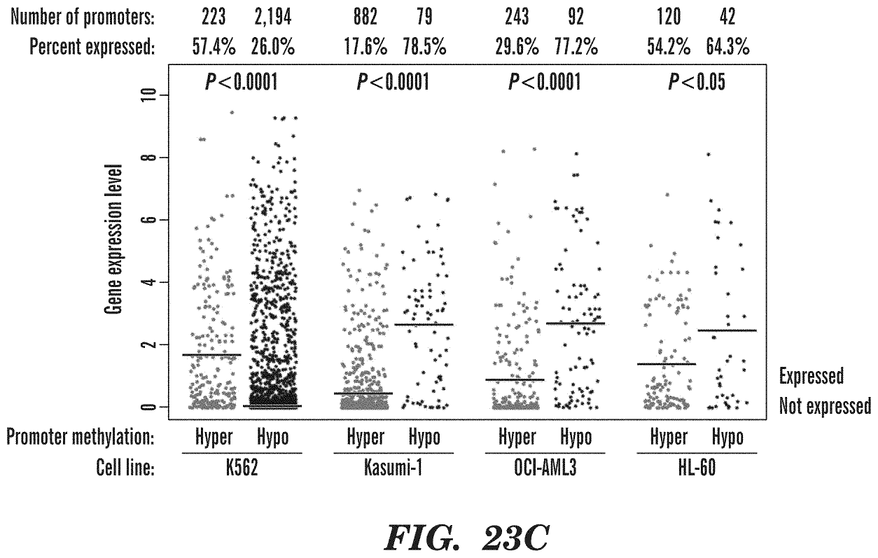

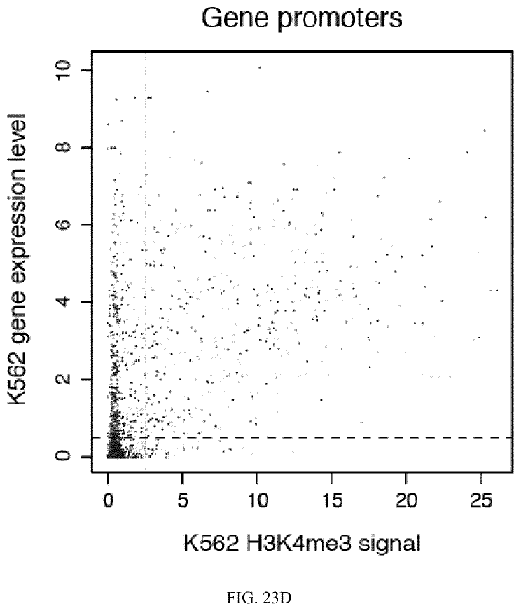

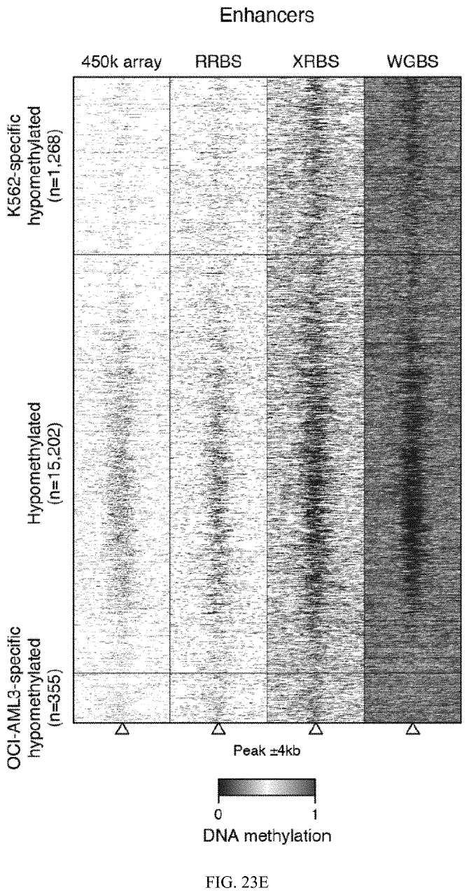

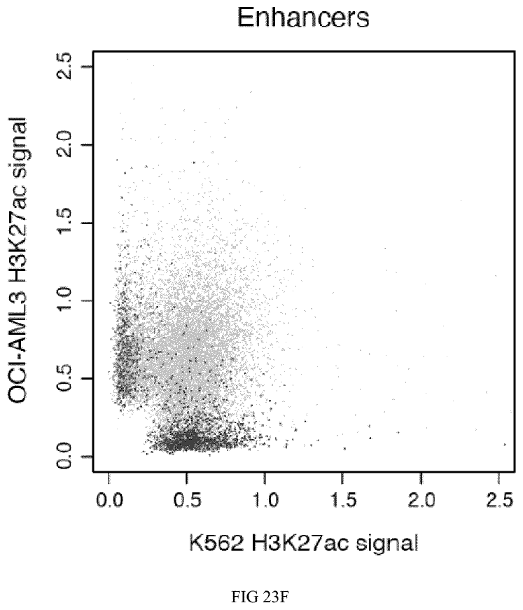

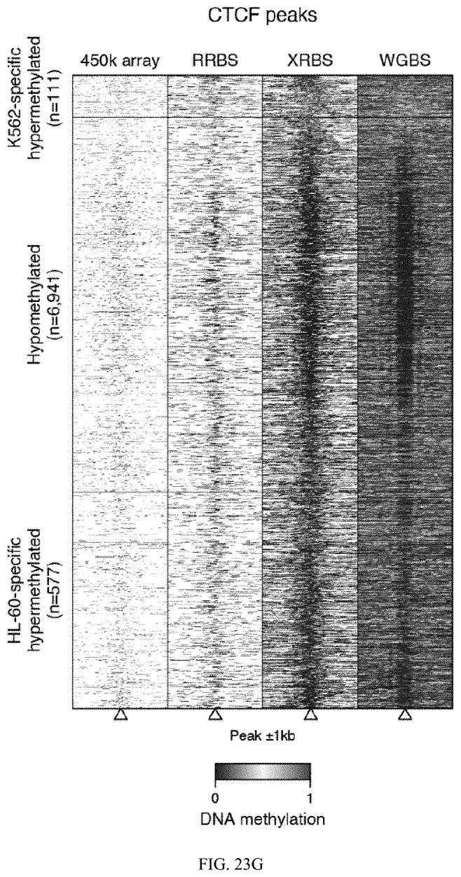

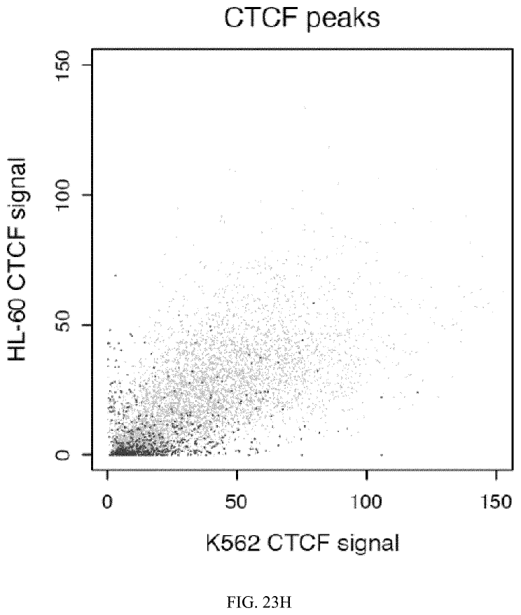

Given the increased coverage of functional elements in expanded representation bisulfite sequencing (XRBS) (FIGS. 15A to 15F), differential methylation was investigated over promoters, enhancers, and CTCF binding sites across cell types. 1,000 cell libraries were deeply sequenced from all four cell lines (FIG. 23A). Of 1,473 promoters that were specifically hypermethylated in a single cell line, the majority were detected in Kasumi-1 (62.0%), in line with its high global DNA methylation levels (FIGS. 17A and 23B). 2,499 promoters were identified that were specifically hypomethylated in a single cell line, with the vast majority detected in the K562 cell line (92.4%). These results were compared with available gene expression datasets. Promoters with cell-specific hypermethylation tended to lack RNA expression in the corresponding cell type (71.4%) (FIG. 23C). Conversely, promoters that were specifically hypomethylated in Kasumi-1, OCI-AML3 or HL-60 tended to be expressed in the corresponding cell type (75.1%). In contrast, most hypomethylated promoters in K562 cells were associated with low expression (74.0%; FIGS. 23C and 23D), consistent with the striking global hypomethylation observed in this cell line (FIGS. 16A to 16G).

It was next determined whether differential DNA methylation over enhancers could inform enhancer activity, using H3K27ac as a surrogate marker. 16,825 H3K27ac peaks from ChIP-seq datasets for K562 and OCI-AML3 cells were aggregated that were covered in an expanded representation bisulfite sequencing (XRBS) dataset. The majority of these enhancers were hypomethylated in both cell lines (90.3%), whereas 7.5% (n=1,268) and 2.1% (n=355) of enhancers were specifically hypomethylated in K562 and OCI-AML3 cells, respectively (FIGS. 17B, 23E, and 23F). Of peaks specifically hypomethylated in OCI-AML3 cells, 98.9% overlapped an H3K27ac peak in OCI-AML3, compared to 3.7% in K562 (P<0.0001, Fisher's exact test). Conversely, of peaks specifically hypomethylated in K562 cells, 86.5% overlapped peaks in K562, compared to 22.6% in OCI-AML3 (P<0.0001). Hence, hypomethylation correlated with H3K27ac, indicating that expanded representation bisulfite sequencing (XRBS) methylation profiling is an efficient method to infer enhancer activity.

Finally, it was determined whether expanded representation bisulfite sequencing (XRBS) could detect differential CTCF binding. Not wishing to be bound by theory, CTCF binding often occurs in CpG sparse regions, but is antagonized by DNA methylation. Differential DNA methylation was evaluated over a merged set of CTCF binding sites from HL-60 and K562 cell lines that were covered in the expanded representation bisulfite sequencing (XRBS) data (n=7,629). A close correspondence was found between cell line-specific hypomethylation and CTCF binding (FIGS. 17C, 23G, and 23H). Of peaks specifically hypermethylated in HL-60 cells (n=577), only 9.9% coincided with a CTCF peak in HL-60, compared to 99.8% in K562 (P<0.0001, Fisher's exact test). Conversely, of peaks specifically hypermethylated in K562 cells (n=111), 52.3% were overlapping a CTCF peak in K562, compared to 88.3% in HL-60 (P<0.001). Hence, expanded representation bisulfite sequencing (XRBS) methylation data can serve as a proxy for CTCF binding.

### Example 11

### Multimodal Single Cell Profiling Using Expanded Representation Bisulfite Sequencing (XRBS)

Key features of expanded representation bisulfite sequencing (XRBS), such as early barcoding and pooled bisulfite conversion, were well-suited for single cell profiling. Multiple single cells were barcoded upfront and combined into a single bisulfite reaction. This could increase sensitivity and reduce variability introduced by bisulfite conversion.

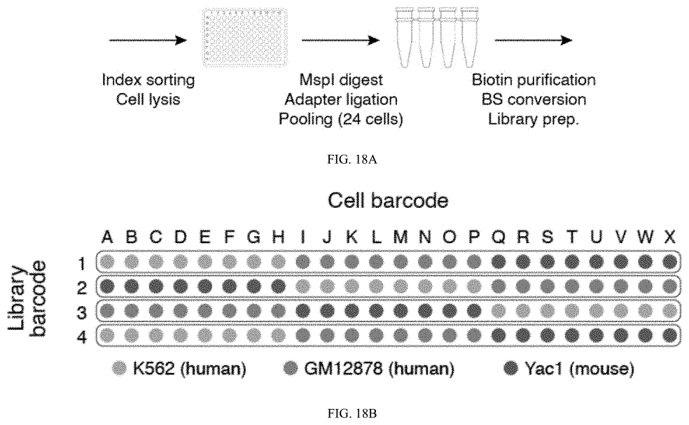

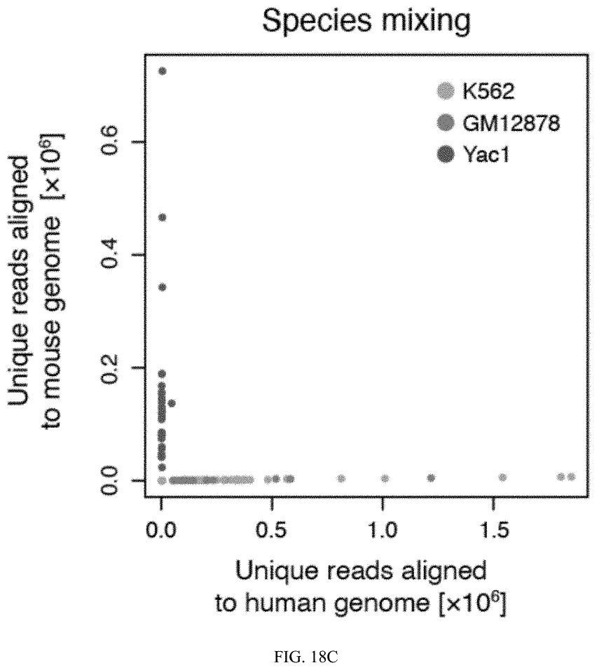

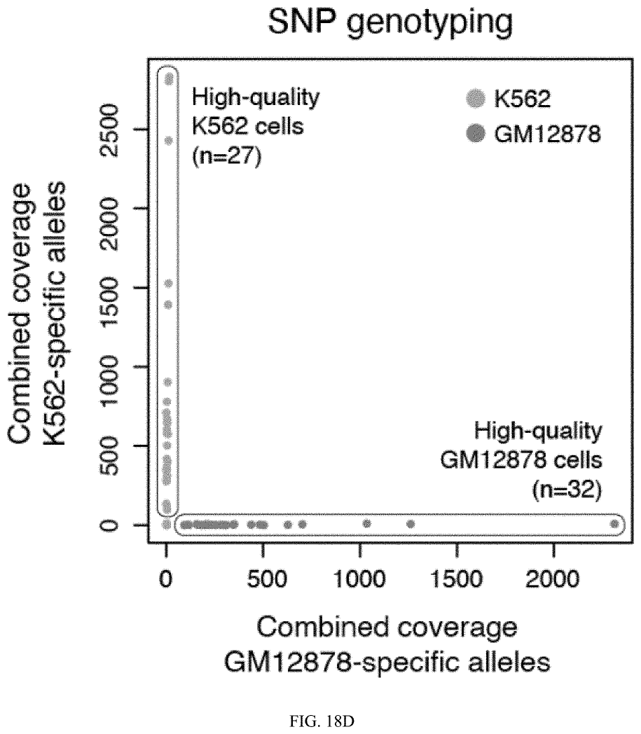

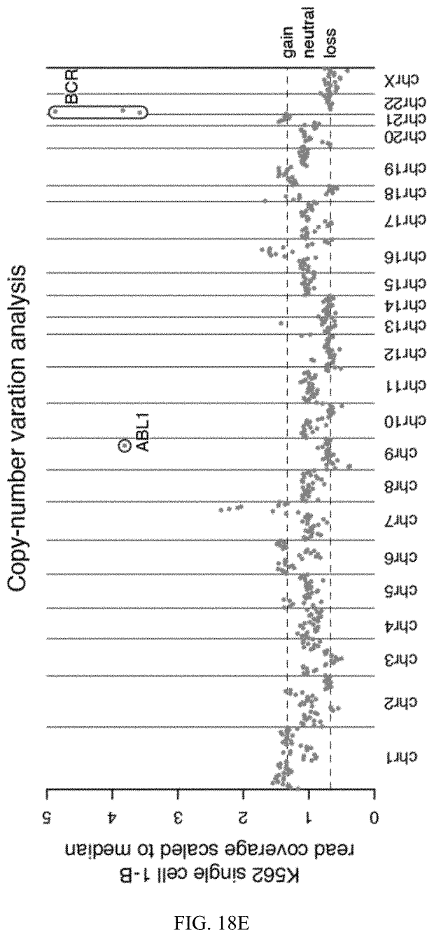

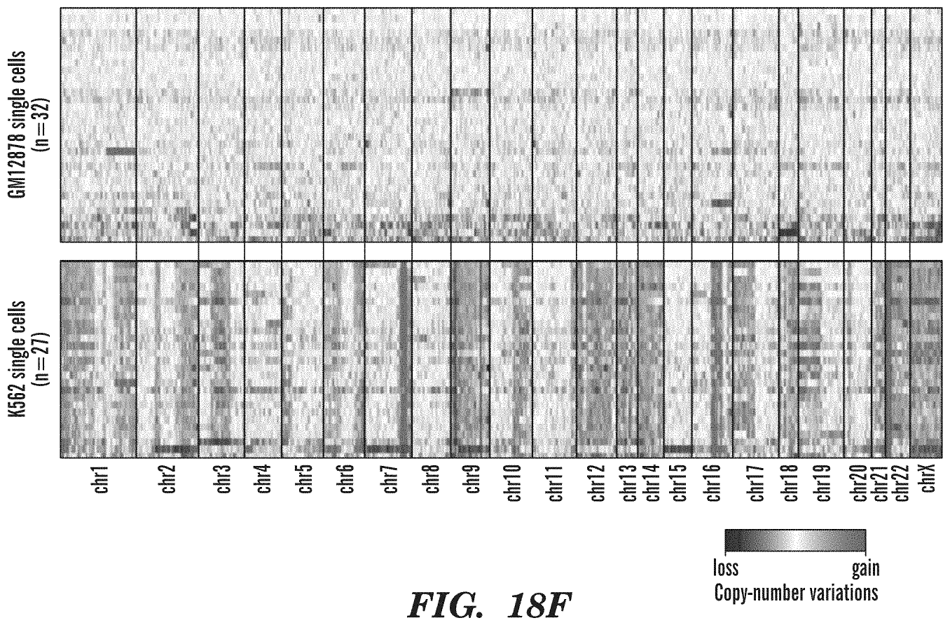

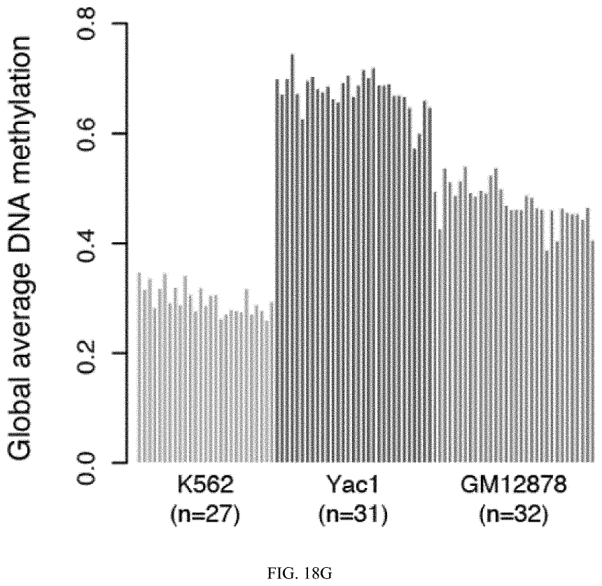

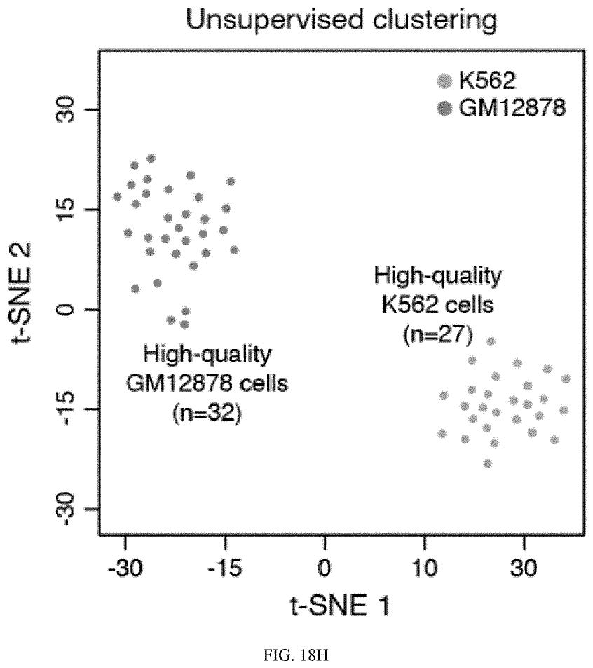

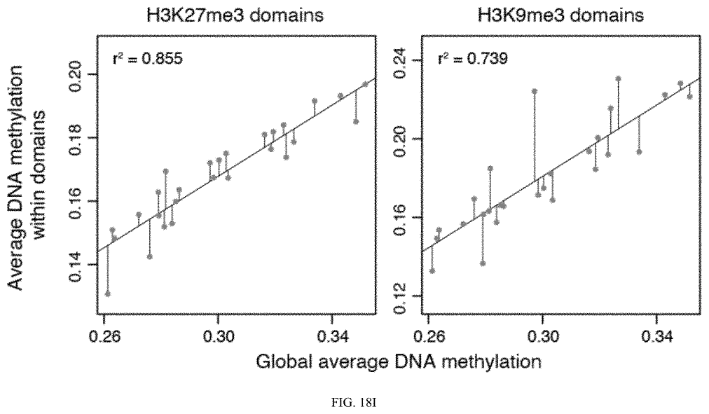

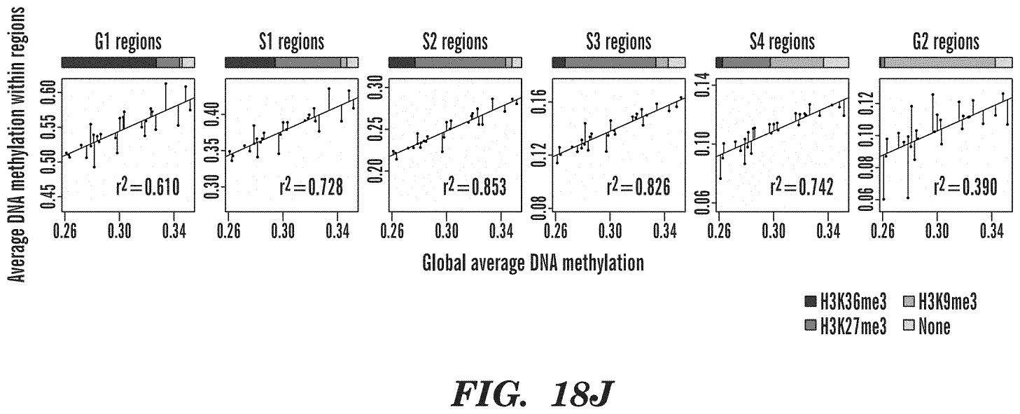

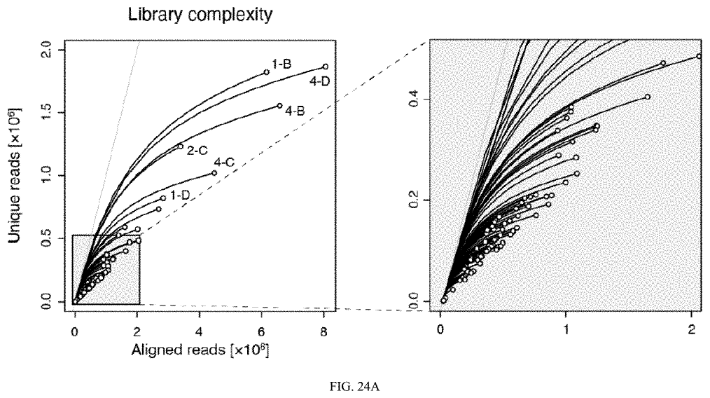

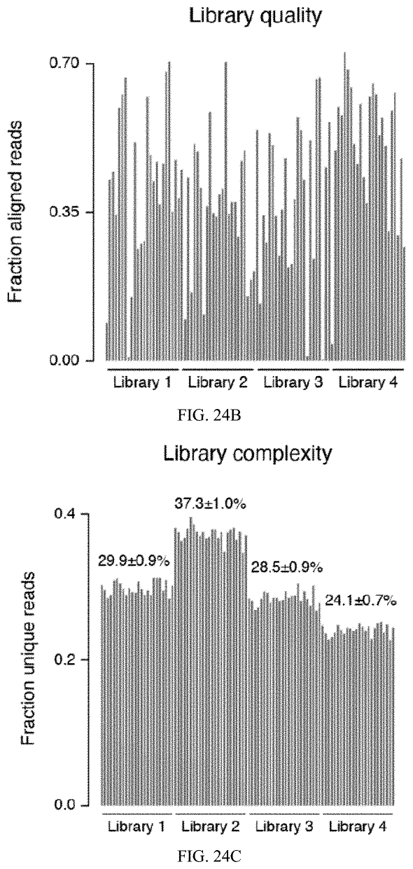

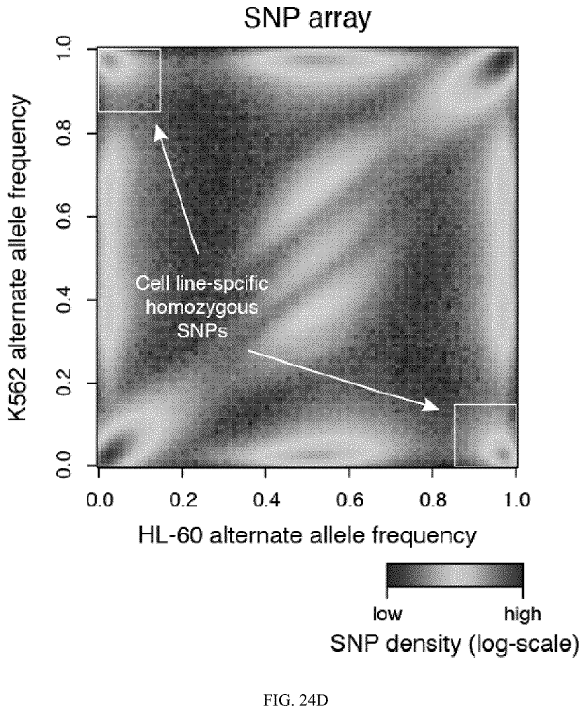

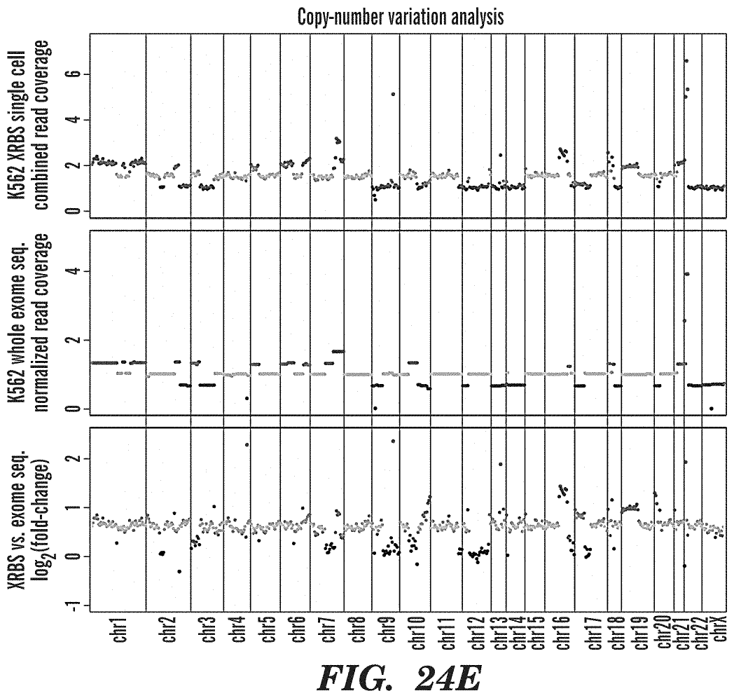

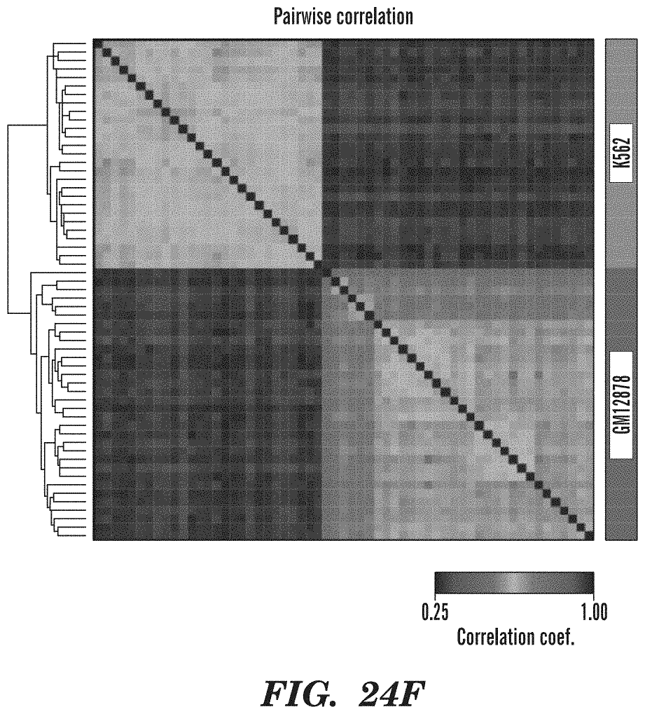

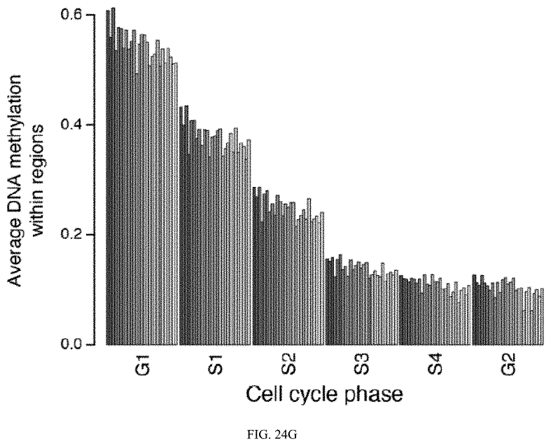

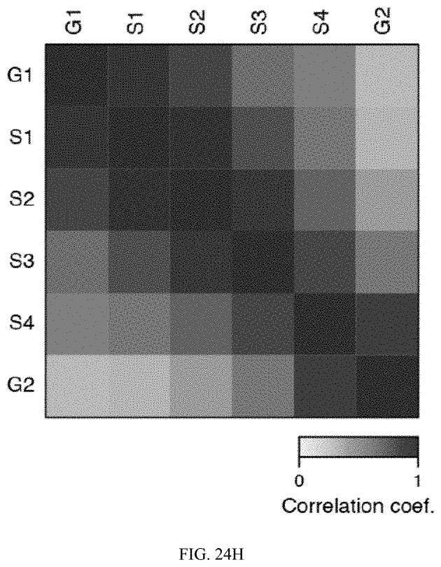

Single cells from human (K562 and GM12878) and mouse (Yac1) cell lines were index sorted into a 96-well plate. In each well, DNA was restricted with MspI and ligated one of 24 cell-identifying barcoded biotinylated adapters (BC1 to BC24 of Table 3). The 96 reactions were then combined into four pools of 24 cells each, adapted fragments were captured on streptavidin beads, and bisulfite conversion was performed (FIGS. 18A and 18B). Next, second strand synthesis and library amplification were carried out with four separate library barcodes to distinguish the pools of 24 cells. Sequencing of these single cell libraries to high depth yielded up to 1.87 million unique reads and 5.29 million CpGs in a single cell profile (FIG. 24A). On average, 42.1±17.7% of expanded representation bisulfite sequencing (XRBS) reads were appropriately aligned to an MspI site (FIG. 24B). An advantageous feature of single cell expanded representation bisulfite sequencing (XRBS), compared to single cell reduced representation bisulfite sequencing (RRBS), was the ability to call PCR duplicates, using both the location of paired-end reads and the sequence of the hexamer as a unique molecular identifier (UMI). It was found that the polymerase chain reaction (PCR) duplication rate was similar across cells within a pool (FIG. 24C).

TABLE 3
Cell-identifying barcoded biotinylated adapters

| | Sequence |
|---|---|
| Adapter barcode name | (first 8 bp of read 2) |
| BC1 | TGGTATAG |
| BC2 | GGATGGTT |
| BC3 | ATAGATTA |
| BC4 | GAAGAGGT |
| BC5 | GAGATTTG |
| BC6 | GGAGGTTG |
| BC7 | ATTAAAGG |
| BC8 | TTTGGTAT |
| BC9 | AAGAGGTT |
| BC10 | TATAATAG |
| BC11 | TTGGTATT |
| BC12 | AAAGTGTT |
| BC13 | GAGTGTGT |
| BC14 | TGGTTAAG |
| BC15 | AAGGATTA |
| BC16 | GTTTAGTA |
| BC17 | TAAATGAT |
| BC18 | TAGATAGG |
| BC19 | GATGGGTT |
| BC20 | GGTAGAGT |
| BC21 | ATGTAATA |
| BC22 | GGTGTAGT |
| BC23 | ATAAAGAT |
| BC24 | TGGGATAT |
| BC25 | TTTAGATT |
| BC26 | TGGAGTGG |
| BC27 | TGAGTAGG |
| BC28 | AAGATGAG |
| BC29 | TGGAGAGA |
| BC30 | AAGGGAAT |
| BC31 | TATGTAAA |
| BC32 | TTAAAGTG |
| BC33 | ATAATTGA |
| BC34 | ATTGAGTG |
| BC35 | GGGTAGAG |
| BC36 | TTAAGTGA |
| BC37 | AGTGTTAT |
| BC38 | TGGTTATA |
| BC39 | TGTTATTA |
| BC40 | GGTAGGGA |
| BC41 | TTGATATG |
| BC42 | AAGTTATA |
| BC43 | AGGTAGGA |
| BC44 | AGAGATGT |
| BC45 | TTGAGGTG |
| BC46 | ATAGGTGA |
| BC47 | GGTGAGGG |
| BC48 | GAAAGTAG |
| BC49 | GGGAATGA |
| BC50 | TAAGTATT |
| BC51 | ATAGAATG |
| BC52 | TAAGTTGA |
| BC53 | GTTAATTT |
| BC54 | TGGTAGAT |
| BC55 | GAATAGTT |
| BC56 | AAGTTTAA |
| BC57 | AGGAAATG |
| BC58 | ATAAGGTA |
| BC59 | AAGGAATT |
| BC60 | AGTTGAAA |
| BC61 | GGAGTGGT |
| BC62 | TTATATAG |
| BC63 | TTTATAAG |
| BC64 | TATATTGG |
| BC65 | TAGGAGGA |
| BC66 | GGTTGATA |
| BC67 | TAGAAGAT |
| BC68 | GGAGTTTA |
| BC69 | ATGGGAGT |
| BC70 | AAGTAAAT |
| BC71 | ATAGGGAT |
| BC72 | GGTGGATT |
| BC73 | GAGGTTGT |
| BC74 | GGGTTAAT |
| BC75 | TAAATGGG |
| BC76 | GTTGGTGA |
| BC77 | GAGTGAAT |
| BC78 | GTTGGGAT |
| BC79 | TTAATTTG |
| BC80 | AGAAAGTG |
| BC81 | ATAGTGAG |
| BC82 | TTTAATGG |
| BC83 | AATAGTAT |
| BC84 | TGAGTGTA |
| BC85 | GGAGATAG |
| BC86 | GAATAAGA |
| BC87 | ATGATAGA |
| BC88 | GTTAGGAG |
| BC89 | GAAGTAGA |
| BC90 | TTAGGTGG |
| BC91 | AGTTAAGA |
| BC92 | AATTAGAA |
| BC93 | TTGAAGGG |
| BC94 | GGATGAAG |
| BC95 | AAGTTGTG |
| BC96 | AAGGGATA |

To evaluate cross contamination between cells or barcodes, mouse and human single cells were evaluated from the same bisulfite reaction. Alignment of single cell expanded representation bisulfite sequencing (XRBS) data to genomes from both species confirmed that barcode cross contamination was extremely rare (FIG. 18C). Furthermore, K562 and GM12878 cells could be distinguished based on homozygous single nucleotide polymorphisms (SNPs) that were unique to each cell line. This information could also be detected in the expanded representation bisulfite sequencing (XRBS) reads, and provided further evidence for minimal cross-contamination (see Materials and Methods for Examples 7 to 11; FIGS. 18D and 24D). The experiment yielded high-quality sequencing data for 27 K562 cells, 32 GM12878 cells and 31 Yac1 cells (6 of the 96 cells were filtered out due to low-quality reads).

In addition to gaining epigenetic and SNP information, single cell expanded representation bisulfite sequencing (XRBS) methylation profiles were used to determine genetic copy-number variations (CNVs) in the same single cells. A computational approach was developed that calculated the relative read coverage in 637 bins across the genome (see Materials and Methods for Examples 7 to 11). Comparing K562 cells to GM12878 cells (which show a predominantly normal karyotype), copy-number variations (CNVs) were detected in the single cell data that were consistent with bulk sequencing data (FIGS. 18E, 18F, and 24E). These included a BCR-ABL amplification characteristic of K562 cells. Individual single cell expanded representation bisulfite sequencing (XRBS) methylation profiles also showed sporadic chromosomal gains and losses that were consistent with genetic instability in both cell lines. Thus, expanded representation bisulfite sequencing (XRBS) profiles provide simultaneous insight into the methylation and activity of functional elements and genetic aberrations in the same single cells.

A challenge in evaluating cell-to-cell variability of DNA methylation relates to the low coverage of single cell methylation data. This was addressed using expanded representation bisulfite sequencing (XRBS). It was found that 93.3±1.1% of CpGs sites covered in a given single K562 or GM12878 cell were also covered in at least one other cell. Average DNA methylation levels were similar across single cells of a given cell type (29.9±2.6%, 67.5±3.5% and 47.5±3.7% for K562, Yac1 and GM12878, respectively; FIG. 18G). Furthermore, single cell methylation profiles from a given human cell type were highly correlated (FIG. 24F), and could be clearly clustered by t-SNE dimensionality reduction analysis (FIG. 18H).

Single cell variability in DNA methylation across different chromatin states was investigated. Relative to global DNA methylation levels, it was found that H3K9me3-marked genomic regions were associated with the highest cell-to-cell variability. In contrast, H3K27me3-marked regions had lower cell-to-cell variability despite having similar average DNA methylation levels (FIG. 18I). Not wishing to be bound by theory, this heterogeneity can be related to replication timing, where late replicating H3K9me3 regions may less accurately maintain their DNA methylation levels. Integration of DNA methylation data with replication timing data for K562 cells confirmed that late replicating regions (“G2 phase”) were indeed the most variable (r²=0.39) and showed the lowest average DNA methylation levels overall (FIGS. 18J, 24G, and 24H). Furthermore, early replicating regions, marked by H3K36me3 and H3K27me3, exhibited less cell-to-cell variability (r²=0.69±0.17) and higher average DNA methylation levels. Not wishing to be bound by theory, these results supported a model in which DNA hypomethylation observed in many cell lines and tumor samples reflects two separate mechanisms: First, hypomethylation in inaccessible, heterochromatic regions may be caused by cumulative loss of DNA methylation at CpGs that have not maintained their parental methylation state before re-entering subsequent cell division. Second, asynchronous sampling of single cells at different timepoints between DNA replication and cell division, during which DNA methylation is propagated to nascent daughter strands, may also significantly contribute to the apparent DNA methylation heterogeneity of late replicating regions in single cell methylomes.

### Materials and Methods for Examples 7 to 11

### Cell Culture

K562, HL-60, and Yac-1 were obtained from ATCC. OCI-AML3 was obtained from DSMZ. All cell lines were routinely tested for mycoplasma contamination and maintained in a 37° C. humidity-controlled incubator with 5.0% CO₂. Cells were maintained in exponential phase growth by passaging every 3 to 4 days. Cells were grown in RPMI supplemented with 10% heat inactivated fetal bovine serum and 1% penicillin/streptomycin.

### DNA Purification

Cells were collected and washed in cold PBS twice and then the pellet was snap frozen in liquid nitrogen. DNA was harvested by thawing the cell pellets at room temperature and resuspending in PBS, as directed by the Qiagen DNA Mini Blood Kit (51104).

### Flow Sorting and Cellular Lysis

For experiments directly performed on sorted cells (1,000, 100, and single cell experiments), cells were counterstained with 1:1000 μL of propidium iodide in phosphate-buffered saline (PBS) and sorted in a Sony SH800 sorter. Viable cells were gated and used for sorting into 96 well plates preloaded with 3 μL of lysis buffer (40 mM Tris-Ac, 1 mM EDTA, and 1 mM DTT). Incubation at 75° C. for 30 minutes was followed by adding 0.5 μL of Qiagen Protease and a 4 hour incubation at 55° C. and a 30 minute incubation at 75° C. to inactivate the proteinase.

### Barcoded Adapters

Adapters consisting of a methylated top strand and biotinylated bottom strand were resuspended in Tris EDTA (TE) buffer at 100 μM, and annealed prior to use. All adapters and primers described herein were obtained from IDT. Briefly, annealing of adapters involved combining equimolar volumes of each adapter and heating to 95° C. 5 mins, followed by slow cooling to 4° C. at a rate of 0.1° C./sec. The methylated top strand contained a partial SBS12 sequence followed by an 8 base pair C-depleted barcode (top strand: 5′-/5SpC3/GG AGT T/iMe-dC/A GA/iMe-dC/ GTG TG/iMe-dC/ T/iMe-dC/T T/iMe-dC//iMe-dC/ GAT /iMe-dC/TD DDD D-3′). The biotinylated bottom strand complemented the methylated top strand with an additional two base pairs at the 5′ end (5′-CG-3′) complementary to the sticky end left by the MspI enzyme (bottom strand: 5′-CGH HHH HHH HAG ATC GGA AGA GCA CAC GTC TGA ACT CC/3Bio/-3′). The barcode sequences used in this study are described in Supplementary Table 3. The 5′ end of the bottom strand was left unphosphorylated, which prevented the formation of a covalent bond to the 3′-OH of a digested DNA fragment. The final ligation product of an adapter to an MspI-digested DNA fragment therefore had a nick between the biotinylated bottom strand adapter and the DNA fragment, facilitating efficient release from streptavidin beads during bisulfite conversion.

### Digestion and Ligation

3 μL of digestion and ligation reagents were added to each well, which held 3 μL of either purified DNA or lysis buffer containing sorted cells. The final reaction contained 10 U MspI enzyme (NEB R0106M), 800 U T4 DNA ligase (NEB M0202M), 1.5 mM ATP (NEB P0756L), 10 nM annealed barcoded adapter, and 1× CutSmart Buffer that was compatible with both MspI restriction enzyme and T4 DNA ligase. The reaction was incubated for 2 hours at 37° C. and 1 hour at 22° C., followed by 4° C. hold. Both digestion of the DNA with MspI and ligation to double stranded adapters occurred concurrently. Adapter sequences were designed such that adapter ligation to digested DNA ends did not result in a new MspI restriction site. When digestion and ligation occurred simultaneously, the equilibrium shifted from intramolecular ligations (between two MspI digested DNA fragments) to intermolecular ligations (MspI DNA and adapter). Adapters were designed with an overhang in order to obviate a fill-in and A-tailing step used in many reduced representation bisulfite sequencing (RRBS) library preparation protocols.

### Streptavidin Bead Binding & Sample Combination

C1 streptavidin beads (Thermo Fisher 65001) were washed with 1× Bind & Wash (B&W) buffer three times according to the kit instructions. Beads were resuspended in 2× B&W buffer and 10 μL were distributed to each reaction. The beads were incubated with barcoded samples on a rotator at room temperature for 15 minutes. For multiplexed single cell libraries, the beads from 24 distinctly barcoded wells were combined and placed in an Eppendorf tube. Using a magnet the beads were separated from the solution containing enzymes and resuspended in 20 μL of water. Resuspended beads were then added to 130 μL of conversion reagent for the bisulfite conversion.

### Bisulfite Conversion

Bisulfite conversion was performed directly on DNA bound to streptavidin beads, using the Zymo DNA Methylation Lightning Kit (D5046) according to the manufacturer's instructions with the following modification: After the initial conversion reaction at 98° C., the reactions were transferred to an Eppendorf tube and the streptavidin beads were pelleted through centrifugation at 4° C. for 10 mins at 16,000 rcf. The supernatant, containing single-stranded, bisulfite-converted DNA with a 5′ barcoded methylated adapter, was separated from the beads and processed further. Bisulfite converted DNA was eluted in 26 μL of water.

### Hexamer Extension and Clean Up

Bisulfite converted single stranded DNA was mixed with 1× NEB Buffer 2.1, 0.4 mM dNTP mix, and 2 μM random hexamer primer (5-TAC ACG ACG CTC TTC CGA TCT NNN NNN-3′). The solution was heated at 95° C. for 45 seconds and then transferred immediately to ice. 1 μL of Klenow enzyme (Enzymatics P7010-HC-L), which has strand displacing activity, was added to each reaction. Hexamer base pairing was mediated through a gradual increase in temperature from a 4° C. incubation and an incremental increase in temperature to 37° C. at a rate of 1° C. per second. Multiple hexamers could bind during this step. Klenow enzyme extended the hexamer primer generating the second strand during a 37° C. incubation for 1.5 hours. Because of the strand displacing activity, the hexamer bound farthest from the MspI site was extended and displaced other shorter hexamer extension products. This resulted in a linear amplification, with single stranded products as well as a double stranded fragment of the longest extension product. A 1× solid phase reversible immobilization (SPRI) was used to remove excess hexamer primer and was eluted in 12 μL of water. 10.5 μL of the elute was used for the final library polymerase chain reaction (PCR). Library fragment size distribution was 150 to 600 basepairs and peaked around 300 base pairs.

### PCR and Clean Up

A final library amplification was carried out with 2× KAPA HiFi U+ mix (Kapa KK2801) and 0.4 μM P5 and P7 PCR primers with 6+14 cycles. 98° C. 1 min; 6× (98° C. 20 sec, 58° C. 30 sec, 72° C. 1 min); 16× (98° C. 20 sec, 65° C. 30 sec, 72° C. 1 min); 72° C. 3 min; 4° C. hold. 1× solid phase reversible immobilization (SPRI) was used to clean up excess library primers. The following primer sequences were used: P7 primer with i7 index: 5-CAA GCA GAA GAC GGC ATA CGA GAT-i7-GTG ACT GGA GTT CAG ACG TGT GC TCT T-3′; P5 primer without i5 index: 5′-AAT GAT ACG GCG ACC ACC GAG ATC TAC ACT CTT TCC CTA CAC GAC GCT CTT CCG ATC T-3′; P5 primer with i5 index: 5′-AAT GAT ACG GCG ACC ACC GAG ATC TCA C-i5-AC ACT CTT TCC CTA CAC GAC GCT CTT CCG ATC T-3′. Index sequences used in this study are provided in Table 4.

TABLE 4
Index sequences.

| i7 | i5 |
|---|---|
| AGCATGGA | GCCAAGAA |
| AGGATCTA | GGAGAGTA |
| ATTCCTCT | CAATGCTA |
| CAATAGTC | TCTGTGAA |
| CGCTATGT | |
| GATATCCA | |
| GGTCCAGA | |
| TCGCTAGA | |
| TCTGCAAG | |
| TTCGCTGA | |

### Decitabine Treatment

Influence of decitabine treatment on viability of Kasumi, HL-60, and OCI-AML3 cells was evaluated through a drug dose response curve. Cells were plated and treated for 5 days with half-log concentrations ranging from 10 nM to 3 μM of decitabine. 30,000 Kasumi cells, 10,000 HL60 cells, and 50,000 OCI-AML3 cells were seeded per well of a 96 well plate based on the recommended seeding density of each cell line. The total volume was 100 μL after drug treatment on day 0. At day 3, cells were imaged and an additional 50 μL of 1× drug in RPMI (Roswell Park Memorial Institute) culture media was added and mixed with the cells. At day 5, 50 μL of CellTiter-Glo® reagent was added, followed by an 8 minute incubation on a rotator at room temperature. Luminescence was imaged using a plate reader and data was plotted using PRISM 2.14.

For expanded representation bisulfite sequencing (XRBS) profiles, HL-60 and OCI-AML3 cells were treated with 300 nM decitabine in 6 well plates. Cells were harvested after 5 days and gDNA was extracted using Qiagen DNA Mini Blood Kit (51104). 10 ng of gDNA were used for each library preparation.

### Next-Generation Sequencing and Barcode Splitting

All libraries were sequenced using the Illumina NextSeq 500 platform according to manufacturer's instructions. Paired-end data was generated using either 2×36 or 2×75 sequencing cycles, and split by single-index or dual-index library barcodes using bcl2fastq. The 8 bp sample barcode was located at the beginning of read 2, which was mostly C-to-T converted. The 6 bp random hexamer sequence was located at the beginning of read 1, which was mostly G-to-A converted. Fastq files were first split by expected sample barcodes, allowing for one mismatch. For libraries that contained only a single sample barcode, two mismatches were allowed. For two sequencing runs that showed poor read quality at the beginning of read 2 due to low sequence complexity, all reads for a given library barcode were included. Sample barcode and random hexamer sequence were trimmed from read 1 and read 2, and appended to the read identifier. Subsequently read 1 and read 2 were swapped to ensure compatibility with downstream analysis tools. Resulting fastq files, which are incorporated herein in their entirety by reference for all purposes, were uploaded to GEO/SRA (GSE149954, token: azujmeaezxqdxgr).

### Genome Alignment and DNA Methylation Calling

Before alignment, primer dimers were filtered using Cutadapt version 2.7 and the following parameters:--discard -a GCTCTTCCGATCT. Short read pairs were trimmed using TrimGalore version 0.6.5 and the following parameters: --paired --illumina --nextseq 20. Trimmed sequencing reads were then aligned to an in-silico bisulfate-converted reference genome (hg38 and mm10) using methylCtools version 1.0.0 and bwa mem version 0.7.17. Sorted alignments were further processed to only maintain uniquely mapped read pairs with a mapping score ≥1, that were mapping to an MspI cut site, and that had an insert size between 20 bp and 600 bp. Putative PCR duplicates were removed by considering the outer mapping position of both paired-end reads, as well as the random hexamer sequence that was trimmed prior to alignment and functioned as a unique molecular identifier (UMI). For library complexity analysis, alignments were downsampled prior to this step. DNA methylation calling was performed using methylCtools bcall and the --trimPE parameter. DNA methylation values, which are incorporated herein by reference for all purposes, were deposited on GEO (GSE149954, token: azujmeaezxqdxgr).

### Visualization and Differential DNA Methylation Analysis

All downstream analyses were performed in R (version 3.6.2), making extensive use of the data.table and GenomicRanges packages. For many analyses, average DNA methylation values within genomic regions were used. Regions included CpG islands, promoters (1 kb up- and downstream of all transcription start sites of protein-coding genes), enhancers and CTCF binding sites (Encyclopedia of DNA Elements (ENCODE) narrowPeak files), and genomic 100 kb-windows. Average methylation values within these regions were calculated by summarizing calls for methylated and unmethylated CpGs across the entire region, and then calculating a single beta-value. For 100 kb-windows, CpGs within islands were not included. Minimum coverage thresholds were applied as indicated. Similarly, average methylation values were calculated for each genomic bin when visualized as heatmaps (genomic regions were separated in 100 equally-sized bins). Differential DNA methylation analysis was performed by sorting regions according to their difference in average methylation values (for enhancers and CTCF binding sites) or by applying a threshold (0.5) on the difference between the average methylation values of one cell line to the average of the other three cell lines (for promoters).

### External Datasets

Whole-bisulfite sequencing data (whole genome bisulfite sequencing (WGBS)) and reduced-representation bisulfite sequencing data (reduced representation bisulfite sequencing (RRBS)) was downloaded as raw fastq files and processed similar to expanded representation bisulfite sequencing (XRBS) datasets to allow for a direct comparison. Hi-C datasets were downloaded as hic files and processed using Juicer tools to generate eigenvectors at 100 kb resolution. For comparison to Hi-C eigenvectors, other datasets were converted to the hg19 assembly using the liftOver tool. All other analyses were performed using the hg38 assembly.

### Single-Cell Genotyping Analysis

Single-nucleotide polymorphisms (SNPs) were identified that were homozygous for different alleles in K562 and GM12878 cell lines. For this purpose, Illumina Infinium Omni5Exome-4 data was obtained from Encyclopedia of DNA Elements (ENCODE) and allele frequencies were calculated using the GenomeStudio software. 153,326 SNPs were identified that distinguished the two cell lines (allele frequency smaller 0.15 or larger 0.85, shown in FIG. 24D). These positions were assessed in the genome alignments of each single cell using samtools mpileup (version 1.8) and the following parameters: -v -t DP,AD --max-depth 1000000. Resulting vcf-files were further processed in R (version 3.6.2) matching allele counts from sequencing data to annotated genotypes. Allele counts were filtered that were not informative because of the C-to-T conversion of unmethylated cytosines during bisulfite treatment. This filtering step was performed taking into account the originating genomic strand, thereby making full use of the information that is contained in the data. Finally, K562- and GM12878-specific allele counts were summarized for each single cell (shown in FIG. 18D).

### Single-Cell Copy-Number Variation Analysis

An approach was developed that uses read coverage at MspI restriction sites to estimate copy-number variations (CNVs) in single cells. For this purpose, genomic bins were generated based on the combined read coverage of the predominantly copy-number neutral GM12878 cells (32 cells). Each chromosome was separated into bins that each had a combined coverage of ˜10,000 reads. Across all chromosomes, a total of 637 bins were generated. Read coverage within bins was then quantified for each single cell from the K562 and GM12878 cell lines relative to the exact combined coverage in GM12878 cells. Individual copy-number variation (CNV) profiles were further normalized by the total read coverage in each single cell (shown in FIGS. 18E and 18F). The validity of this approach was verified by comparing results for the K562 cell lines to published copy-number data based on exome-sequencing. While a number of chromosomal regions showed alternating copy-number states between the two approaches, this likely reflects differences in the cells used for these analyses, as other copy-number variations were detected at high resolution (for example in chromosome 1, shown in FIG. 24E).

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for polynucleotide methylation profiling, the method comprising:
(a) contacting a double stranded polynucleotide with an endonuclease and a ligase in the presence of a double stranded adapter, wherein the adapter top strand comprises a barcode, at least a partial sequencing primer binding site, and one or more methylated cytosines, and the bottom strand of the adapter is at least partially complementary to the top strand, and further comprises a 5′ overhang, and a first member of a binding pair, wherein the contacting generates an adapter ligation product;
(b) converting cytosines present in the adapter ligation product to uracils and isolating the adapter ligation product by contacting the first member of a binding pair with a second member of a binding pair;
(c) contacting the adapter ligation product of (b) with a polymerase and one or more primers comprising a random sequence and at least a partial sequencing primer binding site, thereby generating linear amplicons;
(d) contacting the linear amplicons of (c) with a polymerase and forward and reverse amplification primers, thereby generating amplicons; and
(e) characterizing the amplicons of (d), thereby generating a polynucleotide methylation profile.

2. The method of claim 1, wherein the double stranded polynucleotide is DNA.

3. The method of claim 1, wherein the forward amplification primer comprises a random hexamer fused to at least a partial sequencing primer binding site.

4. The method of claim 3, wherein characterizing the amplicons comprises using the random hexamer to characterize an amplicon as a PCR duplicate.

5. The method of claim 1, wherein the reverse primer comprises a barcode and at least a partial sequencing primer binding site.

6. The method of claim 4, wherein a first sequencing read starts at the random hexamer and a second read starts at a cell-specific barcode, and a first C at the 5′ end is informative.

7. The method of claim 1, wherein the endonuclease activity is dependent on the methylation state of a recognition sequence.

8. The method of claim 1, wherein the endonuclease is MspI.

9. The method of claim 1, wherein the bottom strand of the adapter does not comprise a methylated cytosine.

10. The method of claim 1, wherein the polynucleotide is isolated from a cell in vivo or in vitro.

11. The method of claim 1, wherein step (a) is used to prepare adapter ligation products from two or more distinct double stranded polynucleotides each being from a distinct biological sample, and wherein the adapter ligation products are pooled prior to step (b).

12. A method for characterizing functionally-relevant genomic regions using polynucleotide methylation profiling, the method comprising:
(a) generating a polynucleotide methylation profile according to the method of claim 1, wherein the double stranded polynucleotide comprises genomic DNA from a cell,
(b) determining a methylation state of a functionally-relevant genomic region of the genomic DNA, wherein the functionally-relevant genomic region is selected from the group consisting of promoters, enhancers, CTCF binding sites, and CpG islands, and
(c) characterizing the functionally-relevant genomic regions based upon the methylation state thereof.

13. A method for single cell genomic DNA methylation profiling, the method comprising:
(a) contacting DNA isolated from a single cell with a restriction enzyme and a ligase in the presence of a double stranded adapter under conditions suitable for cleavage of the DNA with the restriction enzyme and ligation of the cleaved ends of the DNA to the adapter, wherein the adapter top strand comprises a cell specific barcode, at least a partial sequencing primer binding site, and one or more methylated cytosines, and the bottom strand of the adapter is at least partially complementary to the top strand, and comprises a 5′ overhang, and a first member of a binding pair, thereby forming an adapter ligation product;
(b) converting cytosines present in the adapter ligation product to uracils and isolating the adapter ligation product by contacting the first member of a binding pair with a second member of a binding pair;
(c) contacting the adapter ligation product with a polymerase, a forward amplification primer comprising a random hexamer fused to at least a partial sequencing primer binding site thereby generating linear amplicons;
(d) contacting the linear amplicons of (c) with a polymerase and forward and reverse amplification primers, thereby generating amplicons; and
(e) sequencing the amplicons of (d) to detect converted bases, wherein a first sequencing read starts at the random hexamer and a second read starts at the cell specific barcode, and the first C at the 5′ end is informative, thereby generating a single cell genomic DNA methylation profile.

14. The method of claim 10, wherein the cell is associated with a disease.

15. The method of claim 14, wherein the disease is cancer, autoimmune disease, Angelman syndrome (AS), Prader-Willi syndrome (PWS), Lynch syndrome, or age related clonal hematopoiesis (ARCH).

16. A method for analyzing genetic variability within a cell population, the method comprising:
(a) preparing DNA methylation profiles for single cells according to the method of claim 13, wherein each of the single cells is from the same cell line, and
(b) comparing the DNA methylation profiles to determine:
(i) genetic copy-number variations among the cells, and/or
(ii) variability in DNA methylation among the cells.

17. A method for single cell genomic DNA methylation profiling, the method comprising:
(a) characterizing a population of cells using flow cytometry;
(b) sorting the cells into individual reaction vessels;
(c) contacting DNA isolated from a single cell with a restriction enzyme and a ligase in the presence of a double stranded adapter under conditions suitable for cleavage of the DNA with the restriction enzyme and ligation of the cleaved ends of the DNA to the adapter, wherein the adapter top strand comprises a cell specific barcode, at least a partial sequencing primer binding site, and one or more methylated cytosines, and the bottom strand of the adapter is at least partially complementary to the top strand, and comprises a 5′ overhang, and a first member of a binding pair, thereby forming an adapter ligation product;
(d) converting cytosines present in the adapter ligation product to uracils and isolating the adapter ligation product by contacting the first member of a binding pair with a second member of a binding pair;
(e) contacting the adapter ligation product with a polymerase, a forward amplification primer comprising a random hexamer fused to at least a partial sequencing primer binding site thereby generating linear amplicons;
(f) contacting the linear amplicons of (e) with a polymerase and forward and reverse amplification primers, thereby generating amplicons; and
(g) sequencing the amplicons of (f) to detect converted bases, wherein a first sequencing read starts at the random hexamer and a second read starts at the cell specific barcode, and the first C at the 5′ end is informative, thereby generating a single cell genomic DNA methylation profile.

18. A method for characterizing DNA methylation of a subject having or suspected of having a disease, the method comprising:
(a) characterizing a population of cells using flow cytometry;
(b) sorting the cells into individual reaction vessels;
(c) contacting DNA isolated from a single cell with restriction enzyme and a ligase in the presence of a double stranded adapter under conditions suitable for cleavage of the DNA with the restriction enzyme and ligation of the cleaved ends of the DNA to the adapter, wherein the adapter top strand comprises a cell specific barcode, at least a partial sequencing primer binding site, and one or more methylated cytosines, and the bottom strand of the adapter is at least partially complementary to the top strand, and comprises a 5′ overhang, and a first member of a binding pair, thereby forming an adapter ligation product;
(d) converting cytosines present in the adapter ligation product to uracils and isolating the adapter ligation product by contacting the first member of a binding pair with a second member of a binding pair;
(e) contacting the adapter ligation product with a polymerase, a forward amplification primer comprising a random hexamer fused to at least a partial sequencing primer binding site thereby generating linear amplicons;
(f) contacting the linear amplicons of (e) with a polymerase and forward and reverse amplification primers, thereby generating amplicons; and
(g) sequencing the amplicons of (f) to detect converted bases, wherein a first sequencing read starts at the random hexamer and a second read starts at the cell specific barcode, and the first C at the 5′ end is informative, thereby characterizing DNA methylation of the subject.

19. A method for polynucleotide methylation profiling, the method comprising:
(a) contacting a double stranded polynucleotide with a transposase in the presence of a double stranded adapter, wherein the adapter top strand comprises a barcode, at least a partial transposase binding site, and one or more methylated cytosines, and the bottom strand of the adapter is at least partially complementary to the top strand, wherein the contacting generates an adapter transposition product;
(b) converting cytosines present in the adapter transposition product to uracils;
(c) contacting the adapter transposition product of (b) with a polymerase and a primer comprising a random sequence, a barcode, and at least a partial sequencing primer binding site, thereby generating linear amplicons;
(d) contacting the linear amplicons of (c) with a polymerase and forward and reverse amplification primers, thereby generating amplicons; and
(e) characterizing the amplicons of (d), thereby generating a polynucleotide methylation profile.

20. A method for bisulfite conversion of a nucleic acid molecule, the method comprising:
contacting a polynucleotide with a double-stranded adapter comprising a top strand comprising one or more methylated cytosines and a bottom strand that lacks methylated cytosines, an MspI enzyme, and a ligase to form a nucleic acid fragment having adapters at its termini; and
contacting the nucleic acid fragment with bisulfite to form a converted nucleic acid fragment.
